(19) United States
(12) Patent Application Publication
(10) Pub. No.: US 2021/0355509 A1
(43) Pub. Date: Nov. 18, 2021

(54) ALTERNATIVE SPLICING REGULATION OF GENE EXPRESSION AND THERAPEUTIC METHODS

(71) Applicant: THE CHILDREN'S HOSPITAL OF PHILADELPHIA, PHILADELPHIA, PA (US)

(72) Inventors: Beverly L. DAVIDSON, Philadelphia, PA (US); Alejandro Mas MONTEYS, Philadelphia, PA (US); Ammiel Al HUNDLEY, Neptune, NJ (US); Paul T. RANUM, Gainesville, FL (US)

(73) Assignee: THE CHILDREN'S HOSPITAL OF PHILADELPHIA, PHILADELPHIA, PA (US)

(21) Appl. No.: 17/265,302

(22) PCT Filed: Aug. 7, 2019
(86) PCT No.: PCT/US2019/045401

§ 371 Date: Feb. 2, 2021

Related U.S. Application Data
(60) Provisional application No. 62/715,756, filed on Aug. 7, 2018.
(60) Provisional application No. 62/837,701, filed on Apr. 23, 2019.
(60) Provisional application No. 62/838,223, filed on Apr. 24, 2019.
(60) Provisional application No. 62/872,417, filed on Jul. 10, 2019.

(51) Int. Cl. C12N 15/90; C12N 15/11; C12N 9/22; A61K 48/00; A61K 38/46; A61K 31/7088; C12N 15/113; A61K 31/519; A61K 31/501; A61K 31/357; C12N 15/86; A61K 9/00; A61K 45/06; A61P 25/28

(52) U.S. Cl. CPC C12N 15/907; C12N 2750/14171; C12N 9/22; A61K 48/0066; A61K 38/465; A61K 31/7088; C12N 15/113; A61K 31/519; A61K 31/501; A61K 31/357; C12N 15/86; A61K 9/0085; A61K 45/06; A61P 25/28; C12N 2310/20; C12N 2800/80; C12N 2310/14; C12N 2320/31; C12N 2320/32; C12N 2750/14143; C12N 15/11

(57) ABSTRACT

Provided herein are chimeric transactivator minigenes, where the alternative splicing of the minigene determines whether a transactivator is expressed. Expression of the transactivator results in the transcription of a target gene that is under the control of a designer promoter sequence. Alternatively, provided herein are chimeric target gene minigenes, wherein the alternative splicing of the minigene directly determines whether the target gene is expressed. The target gene may encode an inhibitory RNA, a CRISPR-Cas9 protein, or a therapeutic protein.

### REFERENCE TO RELATED APPLICATIONS

The present application claims the priority benefit of U.S. provisional application No. 62/872,417, filed Jul. 10, 2019, U.S. provisional application No. 62/838,223, filed Apr. 24, 2019, U.S. provisional application No. 62/837,701, filed Apr. 23, 2019, and U.S. provisional application No. 62/715,756, filed Aug. 7, 2018, the entire contents of each of which is incorporated herein by reference.

### BACKGROUND

### 1. Field

The present disclosure relates generally to the fields of molecular biology and medicine. More particularly, it concerns methods of using alternative splicing regulation to modulate expression of a target gene that encodes, for example, an inhibitory RNA, a therapeutic protein, or a portion of a CRISPR/Cas9 system.

### 2. Description of Related Art

Huntington's disease (HD) is an autosomal dominant neurodegenerative disease that predominantly affects adults, and more rarely, children. HD is part of the family of polyglutamine (polyQ) disorders comprising at least nine different neurodegenerative diseases that result from the expansion of a triplet CAG repeat in specific genes^{1, 2}. Although HTT protein is ubiquitously expressed, the most affected tissue is the brain, with the striatum and the motor cortex impacted early. Patients with HD have progressive neurodegeneration leading to death 10 to 20 years after disease onset². There is no cure for HD, and current treatments are symptomatic³. Exciting early studies using HD animal models demonstrated that disease improved when mutant HTT expression was reduced, even when initiated after disease onset^{4, 5}.

Several methods have been employed to lower HTT levels⁶⁻¹⁰, and among these is RNA interference (RNAi)^{8, 9, 11-14} RNAi is a biological process in which small RNA molecules regulate the expression of specific genes by translation inhibition or mRNA degradation^{15, 16} Scientists have designed different methods to deliver RNAi triggers within the cell. These include small interfering RNAs (siRNAs), short hairpin RNAs (shRNAs) or artificial miRNAs (amiRNAs) (FIG. 1). All have been used to efficiently reduce expression of a target gene after engaging the RNAi pathway. Reports indicate that reducing the levels of toxic polyQ proteins with RNAi ameliorate disease phenotypes using several models of polyglutamine-repeat disease^{8, 11, 12} These studies provide evidence that RNAi-based treatments for neurodegenerative diseases including HD are possible. Short term (6 weeks) data in nonhuman primates (NHPs) and other's long term (6 months) data in NHPs suggest that sustained RNAi therapy is safel^{17, 18}.

However, repeated or life-long application of RNAi therapy to patients requires consideration that silencing of unintended genes and sustained co-opting of the cellular RNAi pathway may induce toxicity over time. Off-target silencing occurs from the interaction of the RNAi sequence with unintended mRNA transcripts that are fully or partially complementary¹⁹⁻²¹. While standard search algorithms can reduce the likelihood of fully complementary off-sequence silencing, it is difficult to avoid unintended silencing that occurs when there is partial complementarity of the expressed RNAi moiety with another sequence, causing miRNA-like repression^{19, 22}. In addition to off-target silencing, co-opting of the RNAi pathway can saturate the cellular RNAi machinery and obstruct endogenous miRNA regulation, causing toxicity²³. This toxicity can be minimized when triggers are delivered as artificial miRNA sequences, which are more efficiently processed than shRNAs^{24, 25} These triggers enter the RNAi pathway before the initial Drosha/DGCR8 processing step¹⁵. The use of a weaker promoter (H1 or ApoE/hAAT promoters) can also reduce this type of toxicity²⁶. While these approaches are promising when tested in cells or in mice, it is difficult to predict if sustained expression from these promoters, for decades, will be safe in humans. As such, an RNAi expression system that can control when and how much exogenous RNAi sequences are expressed in cells is highly favored over constitutive expression platforms. This regulated system is especially relevant for human diseases for which gene silencing is required for the lifetime of the individual.

### SUMMARY

The invention takes advantage of alternative splicing of pre-mRNAs as a mechanism to regulate gene expression. Splicing of pre-mRNAs is a posttranscriptional regulatory process that removes introns and generates protein diversity with alternative inclusion or exclusion of protein-coding exons, parts of exons, and alternative 5′ and 3′ noncoding exons. This alternative exon splicing can be used as a regulatory switch to control the production of specific proteins, such as a mammalian transactivator that is designed to bind to an upstream designer promoter sequence for non-coding RNA (e.g., siRNA) or protein production. This invention provides an innovative method for regulating non-coding RNA and protein expression in mammalian cells and subjects such as humans including, for example, humans with neurodegenerative diseases such as Huntington's disease and spinocerebellar ataxias and humans with a genetic deficiency such as a deficiency in tripeptidyl peptidase 1 (TPP1).

Accordingly, the invention provides methods of controlling expression (i.e., modulating expression, such as increasing or decreasing expression) of non-coding RNAs and proteins, in cells as well as in subjects, including mammalian cells and subjects.

In one embodiment, methods include administering to a cell: a 1^{st} expression cassette comprising a chimeric gene operably linked to a 1^{st} expression control element, wherein the chimeric gene comprises a first portion comprising an alternatively spliced minigene and a second portion that encodes an RNA that encodes the protein, wherein expression of the protein is controlled by the alternative splicing of the first portion, thereby providing and/or controlling expression of a protein.

In another embodiment, methods of providing a protein include administering to subject: a 1^{st} expression cassette comprising a chimeric gene operably linked to a 1^{st} expression control element, wherein the chimeric gene comprises a first portion comprising an alternatively spliced minigene and a second portion that encodes an RNA that encodes the protein, wherein expression of the protein is controlled by the alternative splicing of the first portion.

The invention further provides methods of treating disease states, such as neurodegenerative diseases, diseases caused by genetic defects, or disease caused by deficiencies in gene expression.

In one embodiment, methods of treating a disease in a mammal include administering to the mammal: a 1^{st} expression cassette comprising a chimeric gene operably linked to a 1^{st} expression control element, wherein the chimeric gene comprises a first portion comprising an alternatively spliced minigene and a second portion that encodes an RNA that encodes a protein, wherein expression of the protein is controlled by the alternative splicing of the first portion.

In some embodiments, the second portion that encodes the RNA that encodes the protein includes a translation stop codon, lacks an initiation or start codon, is not an open reading frame to produce the protein, or encodes only a portion of the protein. In some embodiments, alternative splicing of the first portion modifies the transcript thereby deleting or nullifying the stop codon, introducing an initiation or start codon, restoring the open reading frame, or providing a missing portion of the protein.

In some embodiments, the first portion is 5′ of the second portion. In some embodiments, the first portion includes an in-frame translation stop codon. In some embodiments, alternative splicing of the first portion removes the translation stop codon.

In some embodiments, the protein is a transactivator protein. In some embodiment, the protein is not a reporter protein.

In one embodiment, methods include administering to a cell: a 1^{st} expression cassette comprising a chimeric gene operably linked to a 1^{st} expression control element, wherein the chimeric gene comprises a first portion comprising an alternatively spliced minigene and a second portion that encodes a transactivator protein that binds to a 2^{nd} expression control element, wherein expression of the transactivator is controlled by the alternative splicing of the first portion; and a 2^{nd} expression cassette comprising a nucleic acid sequence encoding an RNA operably linked to the 2^{nd} expression control element that the transactivator protein binds, thereby increasing expression of the RNA in the mammalian cell.

In another embodiment, methods of controlling expression of an RNA or a protein include administering to subject: a 1^{st} expression cassette comprising a chimeric gene operably linked to a 1^{st} expression control element, wherein the chimeric gene comprises a first portion comprising an alternatively spliced minigene and a second portion that encodes a transactivator protein that binds to a 2^{nd} expression control element, wherein expression of the transactivator is controlled by the alternative splicing of the first portion; and a 2^{nd} expression cassette comprising a nucleic acid sequence encoding an RNA operably linked to the 2^{nd} expression control element that the transactivator protein binds, thereby increasing expression of the RNA in the subject.

In one embodiment, methods of treating a disease in a mammal include administering to the mammal: a 1^{st} expression cassette comprising a chimeric gene operably linked to a 1^{st} expression control element, wherein the chimeric gene comprises a first portion comprising an alternatively spliced minigene and a second portion that encodes a transactivator protein that binds to a 2^{nd} expression control element, wherein expression of the transactivator is controlled by the alternative splicing of the first portion; or a 2^{nd} expression cassette comprising a nucleic acid sequence encoding an RNA operably linked to the 2^{nd} expression control element that the transactivator protein binds, thereby increasing expression of the RNA in the mammal and treating the disease.

In some embodiments, the RNA is an inhibitory RNA, such as, for example, and siRNA, shRNA, or miRNA. In some embodiments, the inhibitory RNA inhibits or decreases expression of an aberrant or abnormal protein associated with a disease, thereby treating the disease.

In some embodiments, the RNA encodes a therapeutic protein. In some embodiments, the therapeutic protein corrects a protein deficiency associated with a disease, thereby treating the disease.

In some embodiments, wherein the RNA encodes a Cas9 protein. In some embodiments, the methods further comprise administering to the subject a 3^{rd} expression cassette comprising a nucleic acid sequence encoding a guide RNA operably linked to a 3^{rd} expression control element. In some embodiments, the 3^{rd} expression control element is a constitutive promoter. In some embodiments, expression of the Cas9 protein and guide RNA corrects a genetic disease. In some embodiments, the Cas9 protein lack nuclease function, wherein expression of the Cas9 protein and the guide RNA inhibits the expression of a gene.

In some embodiments, the 1^{st} expression control element is a constitutive promoter, a cell-type specific promoter, or an inducible promoter.

In some embodiments, the first portion of the 1^{st} expression cassette and the second portion of the 1^{st} expression cassette are separated by a cleavable peptide. In some embodiments, the cleavable peptide is a self-cleaving peptide, a drug-sensitive protease, or a substrate for an endogenous endoprotease.

In some embodiments, the splicing of the alternatively spliced minigene is regulated by a small molecule splicing modifier. In some embodiments, the splicing of the alternatively spliced minigene is regulated by a disease state in a cell. In some embodiments, the splicing of the alternatively spliced minigene is regulated by a cell type or tissue type.

In some embodiments, increased expression of the transactivator or the protein is provided by inclusion of an alternatively spliced exon in the first portion of the chimeric gene. In some embodiments, the included exon comprises translation initiation regulatory sequences.

In some embodiments, increased expression of the transactivator or the protein is provided by SMN2 exon 7 inclusion. In some embodiments, inclusion of SMN2 exon 7 is triggered by the presence of a small molecule splicing modifier. In some embodiments, the methods further comprise administering the small molecule splicing modifier to the cell or subject, thereby increasing expression of the RNA or the protein. In some embodiments, the small molecule splicing modifier is

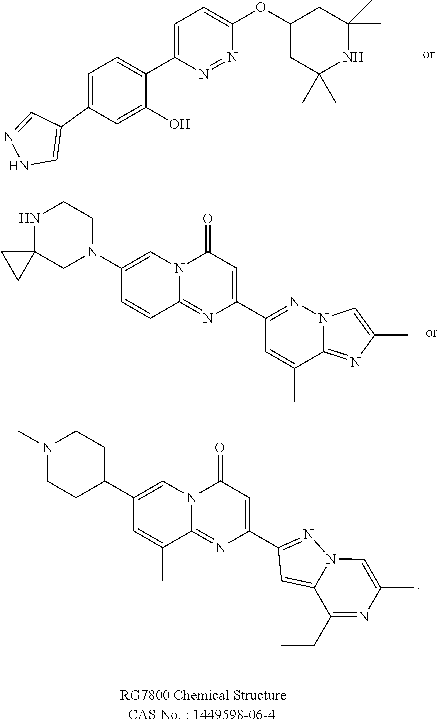

In some embodiments, increased expression of the transactivator or the protein is provided by skipping of an alternatively spliced exon in the first portion of the chimeric gene. In some embodiments, the skipped exon comprises a stop codon. In some embodiments, increased expression of the transactivator or the protein is provided by MDM2 exon 4-11 skipping. In some embodiments, skipping of MDM2 exon 4-11 is triggered by the presence of a small molecule splicing modifier. In some embodiments, the small molecule splicing modifier is sudemycin.

In some embodiments, increased expression of the transactivator is provided by insertion of an exon into a transcript of the transactivator.

In some embodiments, increased expression of the transactivator is provided by skipping of an exon in a transcript of the transactivator.

In some embodiments, an exon is inserted into a transcript that encodes all or a part of a protein, such as a therapeutic protein.

In some embodiments, the exon inserted into the transcript includes a sequence such that when introduced into the transcript the exon restores the protein coding sequence or makes the transcript protein coding sequence in frame. Introduction of the exon into the transcript therefore allows for the complete protein sequence to be encoded by the transcript or the exon provides or restores an open reading frame in the transcript thereby providing or restoring a sequence that translates the protein, for example, a therapeutic protein.

In some embodiments, the exon inserted into the transcript includes a start or initiation codon (e.g., an ATG) absent from the transcript. When the exon is introduced into the transcript in frame this allows translation of the encoded protein, for example, a therapeutic protein.

In some embodiments, the exon inserted into the transcript deletes or nullifies a translation stop codon in the transcript. When the exon is introduced into the transcript deletion or nullification of the translation stop codon allows for translation of the encoded protein, for example, a therapeutic protein.

In some embodiments, the 1^{st} or 2^{nd} expression cassette is comprised in a viral vector.

In various embodiments, the disease is a neuro-degenerative disease. In various embodiments, the neuro-degenerative disease is a poly-glutamine repeat disease. In various embodiments, the poly-glutamine repeat disease comprises Huntington's disease (HD).

In various embodiments, the neuro-degenerative disease is a spinacerebellar ataxia (SCA). In particular aspects, the SCA is any of SCA1, SCA2, SCA3, SCA4, SCA5, SCA6, SCA7, SCA8, SCA9, SCA10, SCA11, SCA12, SCA13, SCA14, SCA16, SCA17, SCA18, SCA19, SCA20, SCA 21, SCA22, SCA23, SCA24, SCA25, SCA26, SCA27, SCA28, or SCA29.

In various embodiments, administration is to the central nervous system (CNS). In particular aspects, administration is to the brain. In particular aspects, administration is to the brain ventricle.

In various embodiments, the 1^{st} or 2^{nd} expression cassette or expression cassette comprising a nucleic acid sequence encoding a protein, comprises a viral vector. In certain aspects, the viral vector is selected from an adeno-associated viral (AAV) vector, a lentiviral vector or a retroviral vector.

In various embodiments, the AAV vector comprises an AAV particle comprising AAV capsid proteins and the 1^{st} or 2^{nd} expression cassette or expression cassette comprising a nucleic acid sequence encoding a protein is inserted between a pair of AAV inverted terminal repeats (ITRs). In particular aspects, the AAV capsid proteins are derived from or selected from the group consisting of AAV1, AAV2, AAV3, AAV4, AAV5, AAV6, AAV7, AAV8, AAV9, AAV10, AAV11, AAV12, AAV-rh74, AAV-rh10 and AAV-2i8 VP1, VP2 and/or VP3 capsid proteins, or a capsid protein having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 VP1, VP2 and/or VP3 capsid proteins. In particular aspects, the one or more of the pair of ITRs is derived from, comprises or consists of an AAV1, AAV2, AAV3, AAV4, AAV5, AAV6, AAV7, AAV8, AAV9, AAV10, AAV11, AAV12, AAV-rh74, AAV-rh10 or AAV-2i8 ITR, or an ITR having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 ITR sequence.

In various embodiments, the 1^{st} or 2^{nd} expression cassette or expression cassette comprising a nucleic acid sequence encoding a protein comprises a promoter.

In various embodiments, the 1^{st} or 2^{nd} expression cassette or expression cassette comprising a nucleic acid sequence encoding a protein comprises an enhancer element.

In various embodiments, the 1^{st} or 2^{nd} expression cassette or expression cassette comprising a nucleic acid sequence encoding a protein comprises a CMV enhancer or chicken beta actin promoter.

In various embodiments, the 1^{st} or 2^{nd} expression cassette or expression cassette comprising a nucleic acid sequence encoding a protein further comprises one or more of an intron, a filler polynucleotide sequence and/or poly A signal, or a combination thereof.

In various embodiments, a plurality of AAV particles are administered.

In some embodiments, AAV particles are administered at a dose of about 1×10⁶ to about 1×10¹⁸ vg/kg.

In some embodiments, AAV particles are administered at a dose from about 1×10⁷-1×10¹⁷, about 1×10⁸-1×10¹⁶, about 1×10⁹-1×10¹⁵, about 1×10¹⁰-1×10¹⁴, about 1×10¹⁰-1×10¹³, about 1×10¹⁰-1×10¹³, about 1×10¹⁰-1×10¹¹, about 1×10¹¹-1×10¹², about 1×10¹²-×10¹³, or about 1×10¹³-1×10¹⁴ vector genomes per kilogram (vg/kg) of the mammal.

In some embodiments, AAV particles are administered at a dose of about 0.5-4 ml of 1×10⁶-1×10¹⁶ vg/ml.

In some embodiments, a method includes in administering a plurality of AAV empty capsids.

In some embodiments, empty AAV capsids are formulated with the AAV particles administered to the mammal. In various aspects, AAV empty capsids are administered or formulated with 1.0 to 100-fold excess of AAV vector particles. In various aspects, AAV empty capsids are administered or formulated with about 1.0 to 100-fold excess of AAV empty capsids to AAV particles.

In some embodiments, administering is intraventricular injection and/or intraparenchymal injection.

In some embodiments, administering is to the brain ventricle, subarachnoid space and/or intrathecal space.

In some embodiments, administering is to neurological cells such as ependymal cells, pial cells, endothelial cells, brain ventricle, meningeal cells, glial cells and/or neurons. In various aspects, the ependymal cell, pial cell, endothelial cell, brain ventricle, meningeal, glial cell and/or neuron expresses the RNAi.

In various embodiments, administration is to the: rostral lateral ventricle; and/or caudal lateral ventricle; and/or right lateral ventricle; and/or left lateral ventricle; and/or right rostral lateral ventricle; and/or left rostral lateral ventricle; and/or right caudal lateral ventricle; and/or left caudal lateral ventricle.

In various embodiments, administration is at a single location in the brain.

In various embodiments, administration is at 1-5 locations in the brain.

In various embodiments, administration is single or multiple doses to any of the mammal's cisterna magna, intraventricular space, brain ventricle, subarachnoid space, intrathecal space and/or ependyma.

In various embodiments, a method reduces an adverse symptom of Huntington's disease (HD) or a spinacerebellar ataxia (SCA). In various aspects, and adverse symptom is an early stage or late stage symptom; a behavior, personality or language symptom; a motor function symptom; and/or a cognitive symptom.

In various embodiments, a method increases, improves, preserves, restores or rescues memory deficits, memory defects or cognitive function of the mammal.

In various embodiments, a method improves or inhibits or reduces or prevents worsening of loss of coordination, slow movement or body stiffness.

In various embodiments, a method improves or inhibits or reduces or prevents worsening of spasms or fidgety movements.

In various embodiments, a method improves or inhibits or reduces or prevents worsening of depression or irritability.

In various embodiments, a method improves or inhibits or reduces or prevents worsening of dropping items, falling, losing balance, difficulty speaking or difficulty swallowing.

In various embodiments, a method improves or inhibits or reduces or prevents worsening of ability to organize.

In various embodiments, a method improves or inhibits or reduces or prevents worsening of ataxia or diminished reflexes.

In various embodiments, a method improves or inhibits or reduces or prevents worsening of seizures or tremors seizures or tremors.

In various embodiments, a mammal is a non-rodent mammal. In various aspects, a non-rodent mammal is a primate. In various aspects, a primate is human. In various aspects, the human is 50 years or older. In various aspects, the human is a child. In various aspects, the child is from about 1 to about 8 years of age.

In various embodiments, a method includes administering one or more immunosuppressive agents. In various aspects, an immunosuppressive agent is administered prior to or contemporaneously with administration of the vector. In various aspects, an immunosuppressive agent is an anti-inflammatory agent.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows co-opting the RNAi pathway for silencing expression in mammalian cells.

FIG. 2 shows action of the regulated promoter system. Inclusion of Exon7 of SMN2 is induced by LMI070, which permits translation of the e6/7/8:transactivator.

FIGS. 3a-b show optimized promoter constructs are responsive to the transactivator. FIG. 3a shows that binding of the transactivator to RNAip induces expression of luciferase. FIG. 3b shows that promoter variants tested (TA, TF2, TF4 and TF5) have minimal expression and are similar to control cells transfected with an empty promoter plasmid (NO), and are fully activated in response to transactivation, as determined by luciferase activity 24 hours after transfection of HEK293 cells.

FIGS. 4a-c show modified SMN2/transactivator minigenes express spliced RNA transcript isoforms to constitutively exclude (CSI3) or include (CSI5) exon7, influencing background expression of the optimized RNAi promoter. FIG. 4a shows that the transactivator was cloned downstream of a self-cleaving 2A peptide and the SMN2 minigene comprising exons 6-7 and the 5′ end of exon 8, and minimal intronic intervening sequences necessary to recapitulate SMN2 splicing. The 3′ and 5′ Exon7 splicing sites in the SMN2/transactivator minigene were modified to constitutively exclude (CSI3, 3′ modified) or include (CSI5, 5′ modified) exon 7. FIG. 4b shows that for CSI, 10% of the transcripts include exon 7, which for CSI3 and CSI5 exon 7 is either included or excluded. FIG. 4c shows that the modification to constitutively exclude exon 7 (CSI3, 3′ modified) minimized RNAi promoter background activation.

FIGS. 5a-b show splicing and activity of CSI and CSI3 minigenes in response to LIM070. FIG. 5a shows that Exon7 inclusion was determined in HEK293 cells by semi-quantitative RT-PCR 20 h after LMI070 treatment. FIG. 5b shows that activation of TF5 RNAi promoter in response to LMI070 was determined by luciferase activity 20 h treatment in HEK293 cells co-transfected with SMN2 minigenes and the TF5 RNAi promoter plasmids.

FIGS. 6a-b show quantitative evaluation of HTT silencing by mi2.4v1, miHDS1v6a and their seed match miRNA controls in HEK293 after transfection. FIG. 6a shows Q-PCR (24 h) and FIG. 6b shows western blot (48 h).

FIG. 7 shows a model of LMI070-regulated RNAi. LMI070 administration will induce miRNA expression and mHTT knockdown (Black, RNAi effect from a single administration of LM1070). RNAi expression should peak 24-48 hours after LMI070 dosing, after which the artificial miRNA will wane to background levels. Predicted RNAi expression over one week after a single (red) or double (blue dashed) LMI070 dose is shown.

FIG. 8 shows HTT de novo splicing in response to LMI070. RNAseq data from HEK293 cells treated with DMSO (CTL) or LMI070 (25 nM). The Sashimi plot depicts the novel minigene (inside the circle in the treated samples) identified by RNA-seq.

FIG. 9 shows beclin 1 (BECN1) de novo splicing in response to LMI070. RNAseq data from HEK293 cells treated with DMSO (CTL) or LMI070 (25 nM). The Sashimi plot depicts the novel minigene (inside the circle in the treated samples) identified by RNA-seq.

FIG. 10 shows chromosome 12 open reading frame 4 (C12orf4) de novo splicing in response to LMI070. RNAseq data from HEK293 cells treated with DMSO (CTL) or LMI070 (25 nM). The Sashimi plot depicts the novel minigene (inside the circle in the treated samples) identified by RNA-seq.

FIG. 11 shows 5′-3′ exoribonuclease 2 (XRN2) de novo splicing in response to LMI070. RNAseq data from HEK293 cells treated with DMSO (CTL) or LMI070 (25 nM). The Sashimi plot depicts the novel minigene (inside the circle in the treated samples) identified by RNA-seq.

FIG. 12 shows splicing factor 3b subunit 3 (SF3B3) de novo splicing in response to LMI070. RNAseq data from HEK293 cells treated with DMSO (CTL) or LMI070 (25 nM). The Sashimi plot depicts the novel minigene (inside the circle in the treated samples) identified by RNA-seq.

FIG. 13 shows formin homology 2 domain containing 3 (FHOD3) de novo splicing in response to LMI070. RNAseq data from HEK293 cells treated with DMSO (CTL) or LMI070 (25 nM). The Sashimi plot depicts the novel minigene (inside the circle in the treated samples) identified by RNA-seq.

FIG. 14 shows glucoside xylosyltransferase 1 (GXYLT1) de novo splicing in response to LMI070. RNAseq data from HEK293 cells treated with DMSO (CTL) or LMI070 (25 nM). The Sashimi plot depicts the novel minigene (inside the circle in the treated samples) identified by RNA-seq.

FIG. 15 shows pyridoxal dependent decarboxylase domain containing 1 (PDXDC1) and nuclear pore complex-interacting protein (PDXDC2P-NPIPB14P), non-coding RNA de novo splicing in response to LMI070. RNAseq data from HEK293 cells treated with DMSO (CTL) or LMI070 (25 nM). The Sashimi plot depicts the novel minigene (inside the circle in the treated samples) identified by RNA-seq.

FIG. 16 shows de novo exon splicing induced by LMI070 (25 nM) in HEK293 cells. PCR confirms the novel exons are included in response to LM1070 treatment (identified by RNA-seq).

FIG. 17 shows XonSwitch strategy 1 used to control gene expression. In the absence of a splice modifier the novel exon is excluded and the downstream protein is out of frame. Thus, no protein is made in this case. After treatment with a splice modifier, the open reading frame is restored and the downstream protein is generated.

FIG. 18 shows XonSwitch strategy 2 used to control gene expression. The upstream ATG translation initiation codon was eliminated and inserted within the novel pseudo-exon. In the absence of a splice modifier, translation will not occur because there is no ATG translation initiation codon present in the transcript, and only a non-protein coding RNA is generated. When a splice modifier is added, the ATG initiation codon is included in the transcript by way of the pseudo-exon which will be translated to express the protein. This provides tight regulation of protein expression.

FIG. 19 shows SMN2 minigene splicing in response to splice modifier RG7800. PCR splicing assay showing induction of Exon 7 inclusion in the SMN2 minigene in response to splice modifier (RG7800) treatment at different doses (10 nM, 100 nM, 1 μM and M).

FIG. 20 shows inducible CRISPR epigenetic silencing by regulated Cas9 expression in response to splice modifier (RG7800) treatment. In the absence of RG7800, Cas9 protein is out of frame and not expressed. Treatment with RG7800 induces Exon7 inclusion and restores the expression of Cas9. As result of Cas9 expression in response to RG7800, a specific Cas9/sgRNA/RBP-Krab complex is formed that binds to the HTT promoter to induce epigenetic silencing of mutant HTT allele.

FIG. 21 shows regulated editing of the mHTT allele. Western blot shows HTT epigenetic silencing induced by an SMN2_CRISPRi regulated system in response to a splice modifier (RG7800). HTT and Cas9 protein levels are shown in HEK293 cells transfected with the SMN2_CRISPRi system after treatment with RG7800 (1 μM). As shown, Cas9 protein levels increase in response to RG7800, and HTT expression levels are reduced about 45% as result of Cas9 mediated epigenetic silencing.

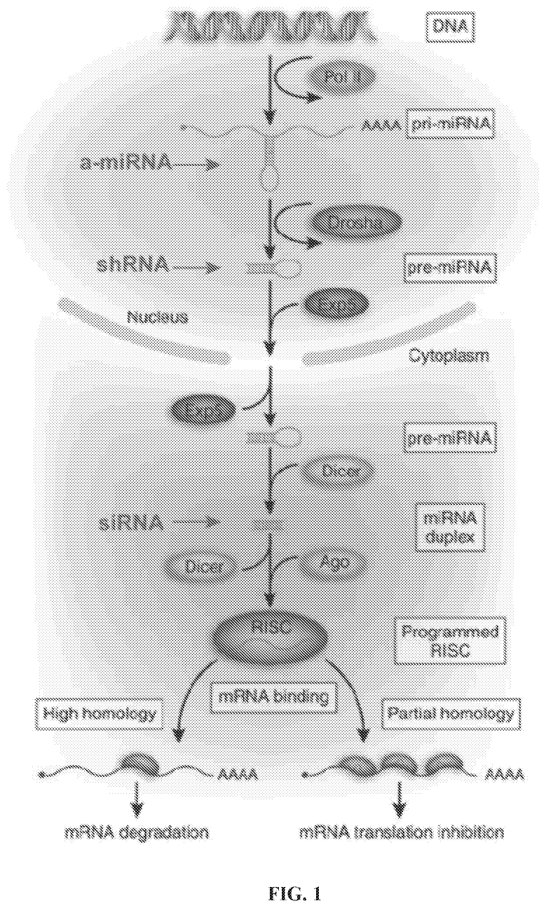

### DETAILED DESCRIPTION

Disclosed herein are chimeric transactivator minigenes, where the alternative splicing of the minigene determines whether the downstream transactivator is expressed. Expression of the transactivator results in the transcription of a target gene that is under the control of a designer promoter sequence. The target gene may encode an inhibitory RNA, a CRISPR-Cas9 protein, or a therapeutic protein.

In one example, the minigene comprises three exons, Exons 1-3, and Exon 2 is skipped in the basal state. When Exon 2 is skipped, the reading frame of Exon 3 is shifted, resulting in the creation of a nonsense mutation in Exon 3. As such, translation of the encoded protein stops in Exon 3, and nothing downstream is translated. Since the transactivator coding sequence is located downstream of the minigene, the transactivator is not expressed in the basal state. Alternatively, translation initiation regulatory sequences are located in Exon 2, and thus when Exon 2 is skipped no translation occurs.

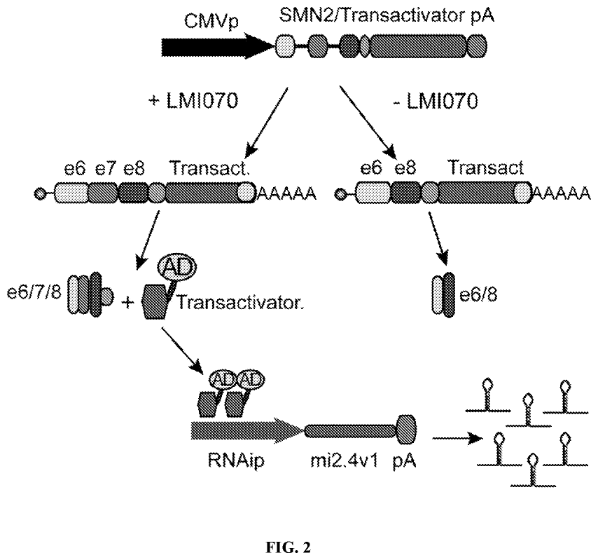

In order to turn on expression of the transactivator, the inclusion of the skipped exon must be induced. Such can occur as a result of the presence of a small molecule splicing modifier. For example, the minigene may comprise Exons 6-8 of the SMN2 gene, in which case Exon 7 is skipped in the basal state. However, Exon 7 is included in the presence of certain splicing modifier small molecules (e.g., LMI070) (see FIG. 2)³¹. As such, the downstream transactivator will be expressed in the presence of LMI070, but not in its absence.

Alternatively, inclusion of the skipped exon may be induced in one cell type, but not another. For example, Exons 8 and 9 of FGFR2 are mutually exclusive, with Exon 9 being only included in mesenchymal tissue (Takeuchi et al., 2010). As such, the minigene may comprise Exons 7, 8, 9, 10 of the FGFR2 gene to allow for expression of the transactivator only in mesenchymal cells. In this case, a stop codon is engineered into Exon 8 of the minigene to prevent transactivator expression in non-mesenchymal cells.

Additional examples of cell type-specific alternative splicing events that may be used in an expression control system of the present disclosure include Eps8 Exon 18B (chr12:15,792,360-15,792,395) and Eps8 Exon18C (chr12:15,787,673-15,787,696), which are specific for auditory hair cells.

As another alternative, inclusion of the skipped exon may be induced by a certain disease state. For example, Huntington's disease results in the generation of transcript isoforms generated by alternative splicing³². As such, the minigene may comprise exons from a gene whose splicing is altered in Huntington's disease, i.e., when mutant HTT is expressed, such that an exon that is normally skipped in a healthy cell is included instead (e.g., PCDH1 (5: 141869432-141878222). If needed, a stop codon may be engineered into the exon downstream of the alternatively spliced exon to ensure that no transactivator is produced in non-diseased cells. The result is that the transactivator will only be expressed when mutant HTT is present. In this example, the target gene may encode an inhibitory RNA that knocks down the expression of mutant HTT, thus creating an autoregulatory feedback loop—the presence of mutant HTT will induce expression of an inhibitory RNA that targets mutant HTT, thereby reducing mutant HTT levels to a level that causes the splicing of the minigene to return to the non-diseased state, thereby turning off the expression of the inhibitory RNA and allowing for expression of mutant HTT, which will reach a level that induces expression of the inhibitory RNA, and so on. Alternatively, the target gene may encode a CRISPR-Cas9 system that represses the transcription of the HTT gene.

In another example, the minigene comprises three exons, Exons 1-3, and Exon 2 is included in the basal state. Inclusion of Exon 2 can either result in a downstream frameshift such that translation stops in Exon 3, or Exon 2 can be engineered to include a stop codon. As such, when Exon 2 is included, i.e., in the basal state, the transactivator is not expressed.

In order to turn on expression of the transactivator, skipping of Exon 2 must be induced. Such can occur as a result of the presence of a small molecule splicing modifier. For example, the minigene may comprise Exons 3, 4, 10, 11, 12 of the MDM2 gene (Singh et al., 2009), which are all included in the basal state. However, Exons 4, 10, 11 are skipped in the presence of certain splicing modifier small molecules (e.g., sudemycin) (Shi et al., 2015). As such, the downstream transactivator will be expressed in the presence of sudemycin, but not in its absence. In this case, a stop codon may be engineered into Exon 4 of the minigene to ensure that no protein is produced in the absence of sudemycin.

Alternatively, skipping of the alternatively included exon may be induced in one cell type, but not another. Exon 18 of Nin is skipped in neurons (Zhang et al., 2016). As such, the minigene may comprise Exons 17, 18, 19 of the Nin gene to allow for expression of the transactivator only in neurons. In this case, a stop codon is engineered into Exon 18 of the minigene to prevent transactivator expression in non-neuronal cells where Exon 18 is included.

As another alternative, skipping of the alternatively included exon may be induced by a certain disease state. For example, Huntington's disease results in the generation of transcript isoforms generated by alternative splicing³². As such, the minigene may comprise exons from a gene whose splicing is altered in Huntington's disease, i.e., when mutant HTT is expressed, such that an exon that is normally included in a healthy cell is skipped instead. A stop codon may be engineered into the alternatively spliced exon to ensure that no transactivator is produced in non-diseased cells. The result is that the transactivator will only be expressed when mutant HTT is present. In this example, the target gene may encode an inhibitory RNA that knocks down the expression of mutant HTT, thus creating an autoregulatory feedback loop—the presence of mutant HTT will induce expression of an inhibitory RNA that targets mutant HTT, thereby reducing mutant HTT levels to a level that causes the splicing of the minigene to return to the non-diseased state, thereby turning off the expression of the inhibitory RNA and allowing for expression of mutant HTT, which will reach a level that induces expression of the inhibitory RNA, and so on. Alternatively, the target gene may encode a CRISPR-Cas9 system that represses the transcription of the HTT gene.

The expression of the chimeric transactivator minigene may be regulated by various types of promoters, depending on the desired expression pattern. For example, the promoter may be a universally constitutive promoter, such as a promoter for a housekeeping gene (e.g., ACTB). As another example, the promoter may be a cell-type specific promoter, such as the promoter for synapsin for neuronal expression. As yet another example, the promoter may be an inducible promoter.

The chimeric transactivator minigene may have a cleavable peptide located between the minigene and the transactivator. In some cases, the cleavable peptide may be a self-cleavable peptide, such as, for example, a 2A peptide. The 2A peptide may be a T2A peptide, a P2A peptide, an E2A peptide, or a F2A peptide. The presence of this peptide provides for separation of the minigene-encoded peptide from the transactivator protein following translation. In some cases, the cleavable peptide may be a cleavage site for a widely expressed, endogenous endoprotease, such as, for example, furin, prohormone convertase 7 (PC7), paired basic amino-acid cleaving enzyme 4 (PACE4), or subtilisin kexin isozyme 2 (SKI-1). In some cases, the cleavable peptide may be a cleavage site for a tissue-specific or cell-specific endoprotease (such as, e.g., prohormone convertase 2 (PC2; primarily expressed in endocrine tissue and brain), prohormone convertase 1/3 (PC1/3; primarily expressed in endocrine tissue and brain), prohormone convertase 4 (PC4; primarily expressed in the testis and ovary), and proprotein convertase subtilisin kexin 9 (PSCK9; primarily expressed in the lung and liver)).

Also disclosed herein are chimeric transactivator minigenes, where the minigene is inserted into the coding sequence of the transactivator, and where the alternative splicing of the minigene determines whether the transactivator is expressed. For example, a MDM2 minigene comprising Exons 4, 10, 11 may be inserted into the coding sequence of the transactivator (Shi et al., 2015). In the basal state, the exons of the minigene are included, thereby disrupting the transactivator coding sequence. In order to ensure that no deleterious protein is produced when the minigene exons are included, the chimeric transactivator minigene may be engineered such that the minigene portion is placed at or near the 5′ end of the transactivator coding sequence and a stop codon may be engineered into Exon 4 of the minigene. In order to induce expression of the transactivator, a splicing modifier molecule (e.g., sudemycin) is used to induce skipping of Exons 4, 10, 11 of the MDM2 minigene. Likewise, cell-type specific or disease state alternative splicing events may be employed as a minigene to be inserted into the coding sequence of the transactivator.

Also disclosed herein are chimeric target gene minigenes, where the minigene is inserted into the coding sequence of the target gene, and where the alternative splicing of the minigene determines whether the target gene is expressed. The target gene may encode an inhibitory RNA, a CRISPR-Cas9 protein, or a therapeutic protein. For example, a MDM2 minigene comprising Exons 4, 10, 11 may be inserted into the coding sequence of the target gene (Shi et al., 2015). In the basal state, the exons of the minigene are included, thereby disrupting the target gene coding sequence. In order to ensure that no deleterious protein is produced when the minigene exons are included, the chimeric target gene minigene may be engineered such that the minigene portion is placed at or near the 5′ end of the target gene coding sequence and a stop codon may be engineered into Exon 4 of the minigene. In order to induce expression of the target gene, a splicing modifier molecule (e.g., sudemycin) is used to induce skipping of Exons 4, 10, 11 of the MDM2 minigene. Likewise, cell-type specific or disease state alternative splicing events may be employed as a minigene to be inserted into the coding sequence of the target gene.

In cases where the chimeric transactivator minigene or chimeric target gene minigene has the minigene inserted at the 5′ end of the transactivator coding sequence or target gene coding sequence, the alternatively included exon may contain necessary translation initiation regulatory sequences. In addition, a cleavable peptide, as described above, may be located between the minigene sequence and the transactivator or target gene.

It is noted that other types of alternative splicing events may be used in the proposed systems as well. For example, a skilled artisan would recognize that an alternative 3′ splice site or alternative 5′ splice site can be engineered to serve the same purpose as an alternatively skipped or included exon. Likewise, a retained intron splicing event can also be engineered accordingly.

### I. TARGET GENES FOR ALTERNATIVE SPLICING REGULATION

A. Inhibitory RNAs

“RNA interference (RNAi)” is the process of sequence-specific, posttranscriptional gene silencing initiated by siRNA. During RNAi, siRNA induces degradation of target mRNA with consequent sequence-specific inhibition of gene expression. Examples of genes whose expression may be inhibited using the expression systems of the present disclosure include, but are not limited to, HTT (for Huntington's disease), SCA (for Spinocerebellar ataxia (type 1, 2, 3, 6, 7), FXTAS (for Fragile X ataxia syndrome), and FMRP (for Fragile X).

An “inhibitory RNA,” “RNAi,” “small interfering RNA” or “short interfering RNA” or “siRNA” molecule, “short hairpin RNA” or “shRNA” molecule, or “miRNA” is a RNA duplex of nucleotides that is targeted to a nucleic acid sequence of interest. As used herein, the term “siRNA” is a generic term that encompasses the subset of shRNAs and miRNAs. An “RNA duplex” refers to the structure formed by the complementary pairing between two regions of an RNA molecule. siRNA is “targeted” to a gene in that the nucleotide sequence of the duplex portion of the siRNA is complementary to a nucleotide sequence of the targeted gene. In certain embodiments, the siRNAs are targeted to the sequence encoding huntingtin. In some embodiments, the length of the duplex of siRNAs is less than 30 base pairs. In some embodiments, the duplex can be 29, 28, 27, 26, 25, 24, 23, 22, 21, 20, 19, 18, 17, 16, 15, 14, 13, 12, 11 or 10 base pairs in length. In some embodiments, the length of the duplex is 19 to 25 base pairs in length. In certain embodiment, the length of the duplex is 19 or 21 base pairs in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24 or 25 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

shRNAs are comprised of stem-loop structures which are designed to contain a 5′ flanking region, siRNA region segments, a loop region, a 3′ siRNA region and a 3′ flanking region. Most RNAi expression strategies have utilized short-hairpin RNAs (shRNAs) driven by strong polIII-based promoters. Many shRNAs have demonstrated effective knock down of the target sequences in vitro as well as in vivo, however, some shRNAs which demonstrated effective knock down of the target gene were also found to have toxicity in vivo.

miRNAs are small cellular RNAs (˜22 nt) that are processed from precursor stem loop transcripts. Known miRNA stem loops can be modified to contain RNAi sequences specific for genes of interest. miRNA molecules can be preferable over shRNA molecules because miRNAs are endogenously expressed. Therefore, miRNA molecules are unlikely to induce dsRNA-responsive interferon pathways, they are processed more efficiently than shRNAs, and they have been shown to silence 80% more effectively.

A recently discovered alternative approach is the use of artificial miRNAs (pri-miRNA scaffolds shuttling siRNA sequences) as RNAi vectors. Artificial miRNAs more naturally resemble endogenous RNAi substrates and are more amenable to Pol-II transcription (e.g., allowing tissue-specific expression of RNAi) and polycistronic strategies (e.g., allowing delivery of multiple siRNA sequences). See U.S. Pat. No. 10,093,927, which is incorporated by reference.

The transcriptional unit of a “shRNA” is comprised of sense and antisense sequences connected by a loop of unpaired nucleotides. shRNAs are exported from the nucleus by Exportin-5, and once in the cytoplasm, are processed by Dicer to generate functional siRNAs. “miRNAs” stem-loops are comprised of sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional siRNAs. “Artificial miRNA” or an “artificial miRNA shuttle vector”, as used herein interchangeably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (˜35 nucleotides upstream and ˜40 nucleotides downstream) arise from restriction enzyme sites within the multiple cloning site of the siRNA. As used herein the term “miRNA” encompasses both the naturally occurring miRNA sequences as well as artificially generated miRNA shuttle vectors.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

In designing RNAi there are several factors that need to be considered, such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits greater than 80%, 85%, 90%, 95%, 98%, or even 100% identity between the sequence of the siRNA and the gene to be inhibited. Sequences less than about 80% identical to the target gene are substantially less effective. Thus, the greater homology between the siRNA and the gene to be inhibited, the less likely expression of unrelated genes will be affected.

In addition, the size of the siRNA is an important consideration. In some embodiments, the present invention relates to siRNA molecules that include at least about 19-25 nucleotides and are able to modulate gene expression. In the context of the present invention, the siRNA is preferably less than 500, 200, 100, 50, or 25 nucleotides in length. More preferably, the siRNA is from about 19 nucleotides to about 25 nucleotides in length.

A siRNA target generally means a polynucleotide comprising a region that encodes a polypeptide, or a polynucleotide region that regulates replication, transcription, or translation or other processes important to expression of the polypeptide, or a polynucleotide comprising both a region that encodes a polypeptide and a region operably linked thereto that regulates expression. Any gene being expressed in a cell can be targeted. Preferably, a target gene is one involved in or associated with the progression of cellular activities important to disease or of particular interest as a research object.

B. CRISPR Systems

Gene editing is a technology that allows for the modification of target genes within living cells. Recently, harnessing the bacterial immune system of CRISPR to perform on demand gene editing revolutionized the way scientists approach genomic editing. The Cas9 protein of the CRISPR system, which is an RNA guided DNA endonuclease, can be engineered to target new sites with relative ease by altering its guide RNA sequence. This discovery has made sequence specific gene editing functionally effective.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. Examples of genes whose expression may be inhibited or whose sequence may be edited using the CRISPR expression systems of the present disclosure include, but are not limited to, HTT (for Huntington's disease), SCA (for Spinocerebellar ataxia (type 1, 2, 3, 6, 7), FXTAS (for Fragile X ataxia syndrome), and FMRP (for Fragile X).

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor (e.g., KRAB) or activator, to affect gene expression. Alternatively, a CRISPR system with a catalytically inactivate Cas9 further comprises a transcriptional repressor or activator fused to a ribosomal binding protein.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence.” In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. The Cas enzyme may be a target gene under the control of a regulated alternative splicing event, as disclosed herein, either as a chimeric target gene minigene or as a target gene for a chimeric minigene transactivator. The gRNA may be under the control of a constitutive promoter.

Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

C. Therapeutic Proteins

Some embodiments concem expression of recombinant proteins and polypeptides. Examples of proteins that may be expressed using the expression systems of the present disclosure include, but are not limited to, STXBP1 (also known as Munc18-1; for STXBP1 deficiency, a form neonatal epilepsy, a form of developmental delay), SCN1a (for Dravet syndrome, also known as genetic epileptic encephalopathy, also known as severe myoclonic epilepsy of Infancy (SMEI); mutations in Nav1.1); SCN1b (mutations in Nav1.1 beta subunit); SCN2b (for familial atrial fibrillation; beta 2 subunit of the type II voltage-gated sodium channel); KCNA1 (for dominantly inherited episodic ataxia; muscle spasms with rigidity with or without ataxia); KCNQ2 (KCNQ2-related epilepsies); GABRB3 (early onset epilepsy; 03 subunit of the GABAA receptor); CACNA1A (for familial ataxias and hemiplegic migraines; transmembrane pore-forming subunit of the P/Q-type voltage-gated calcium channel); CHRNA2 (for autosomal dominant nocturnal frontal lobe epilepsy; alpha subunit of the neuronal nicotinic cholinergic receptor (nAChR)); KCNT1 (for autosomal dominant nocturnal frontal lobe epilepsy (ADNFLE) and malignant migrating partial seizures of infancy (MMPSI); sodium-activated potassium channel); SCN8A (for epilepsy and neurodevelopmental disorders; Nav1.6 deficiency, a voltage-dependent sodium channels); CHRNA4-alpha subunit (for autosomal dominant nocturnal frontal lobe epilepsy; mutation in alpha subunit of nicotinic acetylcholine receptor); CHRNB2-b2 subunit (for autosomal dominant nocturnal frontal lobe epilepsy; mutation in alpha subunit of nicotinic acetylcholine receptor); ARX (for Otohara syndrome, polyAla expansion in ARX gene); MECP1 (for Rett syndrome); FMRP (for Fragile X); and CLN3 (for CLN-disease, also known as Juvenile form of Batten's disease, also known as JNCL). Other examples of proteins that may be expressed using the expression systems of the present disclosure may be found in Lindy et al. (2018) and Heyne et al. (2018), each of which is incorporated herein by reference in its entirety.

In some aspects, the protein or polypeptide may be modified to increase serum stability. Thus, when the present application refers to the function or activity of “modified protein” or a “modified polypeptide,” one of ordinary skill in the art would understand that this includes, for example, a protein or polypeptide that possesses an additional advantage over the unmodified protein or polypeptide. It is specifically contemplated that embodiments concerning a “modified protein” may be implemented with respect to a “modified polypeptide,” and vice versa.

Recombinant proteins may possess deletions and/or substitutions of amino acids; thus, a protein with a deletion, a protein with a substitution, and a protein with a deletion and a substitution are modified proteins. In some embodiments, these proteins may further include insertions or added amino acids, such as with fusion proteins or proteins with linkers, for example. A “modified deleted protein” lacks one or more residues of the native protein, but may possess the specificity and/or activity of the native protein. A “modified deleted protein” may also have reduced immunogenicity or antigenicity. An example of a modified deleted protein is one that has an amino acid residue deleted from at least one antigenic region that is, a region of the protein determined to be antigenic in a particular organism, such as the type of organism that may be administered the modified protein.

Substitution or replacement variants typically contain the exchange of one amino acid for another at one or more sites within the protein and may be designed to modulate one or more properties of the polypeptide, particularly its effector functions and/or bioavailability. Substitutions may or may not be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine, or methionine; serine to threonine; threonine to serine; tryptophan to tyrosine; tyrosine to tryptophan or phenylalanine; and valine to isoleucine or leucine.

In addition to a deletion or substitution, a modified protein may possess an insertion of residues, which typically involves the addition of at least one residue in the polypeptide. This may include the insertion of a targeting peptide or polypeptide or simply a single residue. Terminal additions, called fusion proteins, are discussed below.

The term “biologically functional equivalent” is well understood in the art and is further defined in detail herein. Accordingly, sequences that have between about 70% and about 80%, or between about 81% and about 90%, or even between about 91% and about 99% of amino acids that are identical or functionally equivalent to the amino acids of a control polypeptide are included, provided the biological activity of the protein is maintained. A recombinant protein may be biologically functionally equivalent to its native counterpart in certain aspects.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

As used herein, a protein or peptide generally refers, but is not limited to, a protein of greater than about 200 amino acids, up to a full-length sequence translated from a gene; a polypeptide of greater than about 100 amino acids; and/or a peptide of from about 3 to about 100 amino acids. For convenience, the terms “protein,” “polypeptide,” and “peptide are used interchangeably herein.

As used herein, an “amino acid residue” refers to any naturally occurring amino acid, any amino acid derivative, or any amino acid mimic known in the art. In certain embodiments, the residues of the protein or peptide are sequential, without any non-amino acids interrupting the sequence of amino acid residues. In other embodiments, the sequence may comprise one or more non-amino acid moieties. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid.

Certain embodiments of the present invention concern fusion proteins. These molecules may have a therapeutic protein linked at the N- or C-terminus to a heterologous domain. For example, fusions may also employ leader sequences from other species to permit the recombinant expression of a protein in a heterologous host. Another useful fusion includes the addition of a protein affinity tag, such as a serum albumin affinity tag or six histidine residues, or an immunologically active domain, such as an antibody epitope, preferably cleavable, to facilitate purification of the fusion protein. Non-limiting affinity tags include polyhistidine, chitin binding protein (CBP), maltose binding protein (MBP), and glutathione-S-transferase (GST).

Methods of generating fusion proteins are well known to those of skill in the art. Such proteins can be produced, for example, by de novo synthesis of the complete fusion protein, or by attachment of the DNA sequence encoding the heterologous domain, followed by expression of the intact fusion protein.

Production of fusion proteins that recover the functional activities of the parent proteins may be facilitated by connecting genes with a bridging DNA segment encoding a peptide linker that is spliced between the polypeptides connected in tandem. The linker would be of sufficient length to allow proper folding of the resulting fusion protein.

### II. SPLICING MODIFIERS

A representative splice modifier is LMI070 (Spinraza™, Novartis³¹), which is able to penetrate the blood brain barrier, having the following structure:

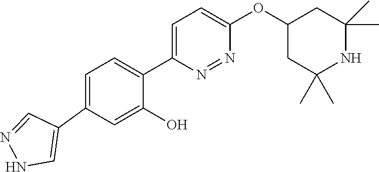 [SMILES: CC1(C)CC(Oc2ccc(-c3ccc(-c4cn[nH]c4)cc3O)nn2)CC(C)(C)N1]

Examples of alternative splicing events where a novel exon is included only in the presence of LMI070, and which can be used for controlling gene expression in the systems of the present disclosure, include, but are not limited to, SF3B3 (chr16:70,526,657-70,529,199), BENC1 (chr17:42,810,759-42,811,797), GXYLT1 (chr12:42,087,786-42,097,614), SKP1 (chr5:134,173,809-134,177,053), C12orf4 (chr12:4,536,017-4,538,508), SSBP1 (chr7:141,739,167-141,742,229), RARS (chr5:168,517,815-168,519,190), PDXDC2P (chr16:70,030,988-70,031,968), STRADB (chr2:201,469,953-201,473,076), WNK1 (chr12:894,562-896,732), WDR27 (chr6:169,660,663-169,662,424), CIP2A (chr3:108,565,355-108,566,638), IFT57 (chr3:108,191,521-108,206,696), WDR27 (chr6:169,660,649-169,662,458), HTT (chr4:3,212,555-3,214,145), SKA2 (chr17:59,112,228-59,119,514), EVC (chr4:5,733,318-5,741,822), DYRKIA (chr21:37,420,144-37,473,056), GNAQ (chr9:77,814,652-77,923,557), ZMYM6 (chr1:35,019,257-35,020,472), CYB5B (chr16:69,448,031-69,459,160), MMS22L (chr6:97,186,342-97,229,533), MEMO1 (chr2:31,883,262-31,892,301), and PNISR (chr6:99,416,278-99,425,413). Examples of alternative splicing events where the inclusion of a novel exon is enhanced by the presence of LMI070, and which can be used for controlling gene expression in the systems of the present disclosure, include, but are not limited to, CACNA2D1 (chr7:82,066,406-82,084,958), SSBP1 (chr7:141,739,083-141,742,248), DDX42 (chr17:63,805,048-63,806,672), ASAP1 (chr8:130,159,817-130,167,688), DUXAP10 (chr14:19,294,564-19,307,199), AVL9 (chr7:32,558,783-32,570,372), DYRKIA (chr21:37,419,920-37,472,960), FAM3A (chrX:154,512,311-154,512,939), FHOD3 (chr18:36,740,620-36,742,886), TBCA (chr5:77,707,994-77,777,000), MZT1 (chr13:72,718,939-72,727,611), LINC01296 (chr14:19,092,877-19,096,652), SF3B3 (chr16:70,541,627-70,544,553), SAFB (chr19:5,654,060-5,654,457), GCFC2 (chr2:75,702,163-75,706,652), MRPL45 (chr17:38,306,450-38,319,088), SPIDR (chr8:47,260,788-47,280,196), DUXAP8 (chr22:15,815,315-15,828,713), PDXDC1 (chr16:15,008,772-15,009,763), MAN1A2 (chr1:117,442,104-117,461,030), RAF1 (chr3:12,600,376-12,604,350), and ERGIC3 (chr20:35,548,787-35,554,452). For the above lists, each genomic location includes the upstream and downstream exon and the intervening intronic sequence targeted by LMI070.

Analogues of splice modifiers such as LMI070 that can be used also are included, for example, 6-(naphthalen-2-yl)-N-(2,2,6,6-tetramethylpiperidin-4-yl)pyridazin-3-amine; 6-(benzo[b]thio-phen-2-yl)-N-methyl-N-(2,2,6,6-tetra-methylpiperidin-4-yl)pyridazin-3-amine; 2-(6-(2,2,6,6-tetramethylpiperidin-4-ylamino)-pyridazin-3-yl)phenol; 2-(6-(methyl-(2,2,6,6-tetra-methylpiperidin-4-yl)amino)pyridazin-3-yl)benzo[b]-thiophene-5-carbonitrile; 6-(quinolin-3-yl)-N-(2,2,6,6-tetramethyl-piperidin-4-yl)pyridazin-3-amine; 3-(benzo[b]-thiophen-2-yl)-6-(2,2,6,6-tetra-methylpiperidin-4-yloxy)pyridazine; 2-(6-(methyl-(2,2,6,6-tetra-methylpiperidin-4-yl)amino)-pyridazin-3-yl)phenol; 6-(6-(methyl-(2,2,6,6-tetra-methylpiperidin-4-yl)amino)-pyridazin-3-yl)naphthalen-2-ol; 6-(benzo[b]-thiophen-2-yl)-N-(2,2,6,6-tetra-methylpiperidin-4-yl)pyridazin-3-amine; 7-(6-((2,2,6,6-tetramethylpiperidin-4-yl)oxy)pyridazin-3-yl)isoquinoline; 6-(6-((2,2,6,6-tetramethylpiperidin-4-yl)oxy)pyridazin-3-yl)isoquinoline; N-methyl-6-(quinolin-7-yl)-N-(2,2,6,6-tetramethyl-piperidin-4-yl)pyridazin-3-amine; N-methyl-6-(quinolin-6-yl)-N-(2,2,6,6-tetramethylpiperidin-4-yl)pyridazin-3-amine; 6-(isoquinolin-7-yl)-N-methyl-N-(2,2,6,6-tetramethylpiperidin-4-yl)pyridazin-3-amine; 6-(isoquinolin-6-yl)-N-methyl-N-(2,2,6,6-tetramethylpiperidin-4-yl)pyridazin-3-amine; 6-(imidazo[1,2-a]pyridin-6-yl-pyridazin-3-yl)-methyl-(2,2,6,6-tetramethyl-piperidin-4-yl)-amine; methyl-[6-(6-phenyl-pyridin-3-yl)-pyridazin-3-yl]-(2,2,6,6-tetramethyl-piperidin-4-yl)-amine; methyl-[6-(6-pyrrol-1-yl-pyridin-3-yl)-pyridazin-3-yl]-(2,2,6,6-tetramethyl-piperidin-4-yl)-amine; methyl-[6-(6-pyrazol-1-yl-pyridin-3-yl)-pyridazin-3-yl]-(2,2,6,6-tetramethyl-piperidin-4-yl)-amine; methyl-(6-quinoxalin-2-yl-pyridazin-3-yl)-(2,2,6,6-tetramethyl-piperidin-4-yl)-amine; methyl-(6-quinolin-3-yl-pyridazin-3-yl)-(2,2,6,6-tetramethyl-piperidin-4-yl)-amine; N-methyl-6-(phthalazin-6-yl)-N-(2,2,6,6-tetramethylpiperidin-4-yl)pyridazin-3-amine; 6-(benzo[c][1,2,5]oxa-diazol-5-yl)-N-(2,2,6,6-tetramethyl-piperidin-4-yl)pyridazin-3-amine; 6-(benzo[d]thiazol-5-yl)-N-(2,2,6,6-tetramethyl-piperidin-4-yl)pyridazin-3-amine; 6-(2-methylbenzo-[d]oxazol-6-yl)-N-(2,2,6,6-tetramethyl-piperidin-4-yl)pyridazin-3-amine; 3-(6-(methyl(2,2,6,6-tetramethylpiperidin-4-yl)amino)pyridazin-3-yl)naphthalen-2-ol; 5-chloro-2-(6-(methyl(1,2,2,6,6-pentamethylpiperidin-4-yl)amino)pyridazin-3-yl)phenol; 3-(6-(2,2,6,6-tetramethylpiperidin-4-ylamino)pyridazin-3-yl)naphthalen-2-ol; 5-chloro-2-(6-(1,2,2,6,6-pentamethylpiperidin-4-ylamino)pyridazin-3-yl)phenol; 4-hydroxy-3-(6-(methyl(2,2,6,6-tetramethylpiperidin-4-yl)amino)pyridazin-3-yl)benzonitrile; 3-[6-(2,2,6,6-tetramethyl-piperidin-4-yloxy)-pyridazin-3-yl]-naphthalen-2-ol; 2-{6-[methyl-(2,2,6,6-tetramethyl-piperidin-4-yl)-amino]-pyridazin-3-yl}-4-trifluoromethyl-phenol; 2-fluoro-6-{6-[methyl-(2,2,6,6-tetramethyl-piperidin-4-yl)-amino]-pyridazin-3-yl}-phenol; 3,5-dimethoxy-2-{6-[methyl-(2,2,6,6-tetramethyl-piperidin-4-yl)-amino]-pyridazin-3-yl}-phenol; 4,5-dimethoxy-2-{6-[methyl-(2,2,6,6-tetramethyl-piperidin-4-yl)-amino]-pyridazin-3-yl}-phenol; 5-methoxy-2-{6-[methyl-(2,2,6,6-tetramethyl-piperidin-4-yl)-amino]pyridazin-3-yl}-phenol; 4,5-difluoro-2-{6-[methyl-(2,2,6,6-tetramethyl-piperidin-4-yl)-amino]-pyridazin-3-yl}-phenol; 5-fluoro-2-{6-[methyl-(2,2,6,6-tetramethyl-piperidin-4-yl)-amino]-pyridazin-3-yl}-phenol; 3-hydroxy-4-(6-(methyl(2,2,6,6-tetramethylpiperidin-4-yl)amino)pyridazin-3-yl)benzonitrile; 1-allyl-6-(6-(methyl(2,2,6,6-tetramethylpiperidin-4-yl)amino)pyridazin-3-yl)naphthalen-2-ol; 6-(benzo[b]thiophen-2-yl)-N-(1,2,2,6,6-pentamethylpiperidin-4-yl)pyridazin-3-amine; N-allyl-3-hydroxy-4-(6-(methyl(2,2,6,6-tetramethylpiperidin-4-yl)amino)pyridazin-3-yl)benzamide; 2-(6-(methyl (2,2,6,6-tetramethylpiperidin-4-yl)amino)pyridazin-3-yl)-5-(1H-pyrazol-1-yl)phenol; 5-(5-methyl-oxazol-2-yl)-2-{6-[methyl-(2,2,6,6-tetramethyl-piperidin-4-yl)-amino]pyridazin-3-yl}-phenol; 5-(4-hydroxymethyl)-1H-pyrazole-1-yl)-2-(6-(methyl(2,2,6,6-tetramethylpiperidin-4-yl)amino)pyridazin-3-yl)phenol; 5-(1H-imidazole-1-yl)-2-(6-(methyl(2,2,6,6-tetramethyl-piperidin-4-yl)amino)pyridazin-3-yl)phenol; 5-(4-amino-1H-pyrazole-1-yl)-2-(6-(methyl(2,2,6,6-tetramethylpiperidin-4-yl)amino)pyridazin-3-yl)phenol; 5-(4-amino-1H-pyrazol-1-yl)-2-(6-(methyl(2,2,6,6-tetramethylpiperidin-4-yl)amino)pyridazin-3-yl)phenol; 5-(3-amino-pyrazol-1-yl)-2-{6-[methyl-(2,2,6,6-tetramethyl-piperidin-4-yl)-amino]pyridazin-3-yl}-phenol; 2-(6-(methyl(2,2,6,6-tetramethylpiperidin-4-yl)amino)pyridazin-3-yl)-5-(1-(2-morpholino-ethyl)-1H-pyrazol-4-yl)phenol; 2-(6-(methyl (2,2,6,6-tetramethylpiperidin-4-yl)amino)pyridazin-3-yl)-5-(1-methyl-1H-pyrazol-4-yl)phenol; 5-(5-amino-1H-pyrazol-1-yl)-2-(6-(methyl-(2,2,6,6-tetramethyl-piperidin-4-yl)amino)pyridazin-3-yl)phenol; 2-(6-(methyl (2,2,6,6-tetramethylpiperidin-4-yl)amino)pyridazin-3-yl)-4-(1H-pyrazol-1-yl)phenol; 2-{6-[(2-hydroxy-ethyl)-(2,2,6,6-tetramethyl-piperidin-4-yl)-amino]pyridazin-3-yl}-5-pyrazol-1-yl-phenol; 2-(6-(piperidin-4-yloxy)pyridazin-3-yl)-5-(1H-pyrazol-1-yl)phenol; 2-(6-(((2S,4R,6R)-2,6-dimethylpiperidin-4-yl)oxy)pyridazin-3-yl)-5-(1H-pyrazol-1-yl)phenol; 2-(6-((-2,6-di methyl piperidin-4-yl)oxy)pyridazin-3-yl)-5-(1H-pyrazol-1-yl)phenol; 2-(6-((-2,6-di methyl piperidin-4-yl)oxy)pyridazin-3-yl)-5-(1H-pyrazol-1-yl)phenol; 5-(1H-pyrazol-1-yl)-2-(6-(pyrrolidin-3-yloxy)pyridazin-3-yl)phenol; 2-(6-((-2-methylpiperidin-4-yl)oxy)pyridazin-3-yl)-5-(1H-pyrazol-1-yl)phenol; (S)-5-(1H-Pyrazol-1-yl)-2-(6-(pyrrolidin-3-ylmethoxy)pyridazin-3-yl)phenol; (R)-5-(1H-pyrazol-1-yl)-2-(6-(pyrrolidin-3-ylmethoxy)pyridazin-3-yl)phenol; 2-(6-((3-fluoropiperidin-4-yl)oxy)pyridazin-3-yl)-5-(1H-pyrazol-1-yl)-phenol; 2-[6-(1,2,2,6,6-pentamethyl-piperidin-4-yloxy)-pyridazin-3-yl]-5-pyrazol-1-yl-phenol; 5-pyrazol-1-yl-2-[6-(2,2,6,6-tetramethyl-piperidin-4-yloxy)-pyridazin-3-yl]-phenol; 5-(1H-Pyrazol-4-yl)-2-(6-((2,2,6,6-tetramethylpiperidin-4-yl)oxy)pyridazin-3-yl)phenol; 2-(6-piperazin-1-yl-pyridazin-3-yl)-5-pyrazol-1-yl-phenol; 3-[6-(azetidin-3-ylamino)-pyridazin-3-yl]-naphthalen-2-ol; 2-[6-(azetidin-3-ylamino)-pyridazin-3-yl]-5-pyrazol-1-yl-phenol; 2-[6-(3,5-di methyl-piperazin-1-yl)-pyridazin-3-yl]-5-pyrazol-1-yl-phenol; 2-[6-(7-methyl-2,7-diaza-spiro[4.4]non-2-yl)-pyridazin-3-yl]-5-pyrazol-1-yl-phenol; 2-(6-[1,4]diazepan-1-yl-pyridazin-3-yl)-5-pyrazol-1-yl-phenol; 2-{6-[4-(2-hydroxy-ethyl)-piperazin-1-yl]-pyridazin-3-yl}-5-pyrazol-1-yl-phenol; 2-[6-(3,6-diaza-bicyclo[3.2.1]oct-3-yl)-pyridazin-3-yl]-5-pyrazol-1-yl-phenol; 2-[6-(2,7-diaza-spiro[3.5]non-7-yl)-pyridazin-3-yl]-5-pyrazol-1-yl-phenol; 2-[6-(3-hydroxy-methyl-piperazin-1-yl)-pyridazin-3-yl]-5-pyrazol-1-yl-phenol; 2-[6-(1,7-diaza-spiro[4.4]non-7-yl)-pyridazin-3-yl]-5-pyrazol-1-yl-phenol; 2-[6-(4-amino-4-methyl-piperidin-1-yl)-pyridazin-3-yl]-5-pyrazol-1-yl-phenol; 2-[6-(3-dimethyl-amino-piperidin-1-yl)-pyridazin-3-yl]-5-pyrazol-1-yl-phenol; 2-[6-(1,2,2,6,6-pentamethyl-piperidin-4-ylamino)-pyridazin-3-yl]-5-pyrazol-1-yl-phenol; 2-[6-(3,3-di methyl-piperazin-1-yl)-pyridazin-3-yl]-5-pyrazol-1-yl-phenol; 2-(6-(7-(2-hydroxyethyl)-2,7-diazaspiro[4.4]-nonan-2-yl)pyridazin-3-yl)-5-(1H-pyrazol-1-yl)phenol; 2-(6-((3aR,6aS)-hexahydropyrrolo[3,4-c]pyrrol-2(1H)-yl)pyridazin-3-yl)-5-(1H-pyrazol-1-yl)phenol; 3-(6-(piperazin-1-yl)pyridazin-3-yl)naphthalene-2,7-diol; 5-pyrazol-1-yl-2-[6-(1,2,3,6-tetrahydro-pyridin-4-yl)-pyridazin-3-yl]-phenol; 2-(6-piperidin-4-yl-pyridazin-3-yl)-5-pyrazol-1-yl-phenol; 3-(6-(1,2,3,6-tetra-hydropyridin-4-yl)pyridazin-3-yl)naphthalen-2-ol; 3-(6-(1,2,3,6-tetrahydropyridin-4-yl)pyridazin-3-yl)naphthalene-2,7-diol; 3-(6-(2,2,6,6-tetramethyl-1,2,3,6-tetrahydropyridin-4-yl)pyridazin-3-yl)naphthalene-2,7-diol; 3-(6-(1-methyl-1,2,3,6-tetrahydropyridin-4-yl)pyridazin-3-yl)naphthalene-2,7-diol; 3-(6-(piperidin-4-yl)pyridazin-3-yl)naphthalene-2,7-diol; 3-(6-((2,2,6,6-tetramethylpiperidin-4-yl)oxy)pyridazin-3-yl)naphthalene-2,7-diol; 3-(6-(methyl(2,2,6,6-tetramethylpiperidin-4-yl)amino)pyridazin-3-yl)naphthalene-2,7-diol; 3-(6-((2,2,6,6-tetramethylpiperidin-4-yl)amino)pyridazin-3-yl)naphthalene-2,7-diol; [3-(7-hydroxy-6-{6-[methyl-(2,2,6,6-tetramethyl-piperidin-4-yl)-amino]-pyridazin-3-yl}-naphthalen-2-yloxy)-propyl]-carbamic acid tert-butyl ester; 7-(3-amino-propoxy)-3-{6-[methyl-(2,2,6,6-tetramethyl-piperidin-4-yl)-amino]-pyridazin-3-yl}-naphthalen-2-ol; N-[3-(7-hydroxy-6-{6[methyl-(2,2,6,6-tetramethyl-piperidin-4-yl)-amino]-pyridazin-3-yl}-naphthalen-2-yloxy)-propyl]-acetamide; 7-(3-hydroxypropoxy)-3-(6-(methyl(2,2,6,6-tetramethylpiperidin-4-yl)amino)pyridazin-3-yl)naphthalen-2-ol; 7-(3-methoxypropoxy)-3-(6-(methyl(2,2,6,6-tetramethylpiperidin-4-yl)amino)pyridazin-3-yl)naphthalen-2-ol; 7-(2-morpholinoethoxy)-3-(6-((2,2,6,6-tetramethylpiperidin-4-yl)oxy)pyridazin-3-yl)naphthalen-2-ol; 3-(6-(piperidin-4-ylmethyl)pyridazin-3-yl)naphthalen-2-ol; 5-(1H-pyrazol-1-yl)-2-(6-((2,2,6,6-tetramethylpiperidin-4-yl)methyl)pyridazin-3-yl)phenol; 3-methoxy-2-(6-(methyl (2,2,6-trimethylpiperidin-4-yl)amino)pyridazin-3-yl)-5-(5-methyloxazol-2-yl)phenol; 2-(6-((6S)-6-((S)-1-hydroxyethyl)-2,2-dimethylpiperidin-4-yloxy)pyridazin-3-yl)-5-(1H-pyrazol-1-yl)phenol; 7-hydroxy-6-(6-(methyl(2,2,6,6-tetramethylpiperidin-4-yl)amino)pyridazin-3-yl)-2-naphthonitrile; 3-(6-(methyl (2,2,6,6-tetramethylpiperidin-4-yl)amino)pyridazin-3-yl)-7-(piperidin-1-ylmethyl)naphthalen-2-ol; 3-(6-(methyl (2,2,6,6-tetramethylpiperidin-4-yl)amino)pyridazin-3-yl)-7-(pyrrolidin-1-ylmethyl)naphthalen-2-ol; 1-bromo-6-(6-(methyl(2,2,6,6-tetramethylpiperidin-4-yl)amino)pyridazin-3-yl)naphthalene-2,7-diol; 1-chloro-6-(6-(methyl(2,2,6,6-tetramethylpiperidin-4-yl)amino)pyridazin-3-yl)naphthalene-2,7-diol; 7-methoxy-3-(6-(methyl(2,2,6,6-tetramethylpiperidin-4-yl)amino)pyridazin-3-yl)naphthalen-2-ol; 7-methoxy-3-(6-(methyl(1,2,2,6,6-pentamethylpiperidin-4-yl)amino)pyridazin-3-yl)naphthalen-2-ol; 7-(3,6-dihydro-2H-pyran-4-yl)-3-(6-(methyl(2,2,6,6-tetramethylpiperidin-4-yl)amino)pyridazin-3-yl)naphthalen-2-ol; 3-(6-(methyl(2,2,6,6-tetramethylpiperidin-4-yl)amino)pyridazin-3-yl)-7-(tetrahydro-2H-pyran-4-yl)naphthalen-2-ol; 7-(difluoromethyl)-3-(6-(methyl(2,2,6,6-tetramethylpiperidin-4-yl)amino)pyridazin-3-yl)naphthalen-2-ol; 7-((4-hydroxy-2-methylbutan-2-yl)oxy)-3-(6-(methyl(2,2,6,6-tetramethylpiperidin-4-yl)amino)pyridazin-3-yl)naphthalen-2-ol; 7-(3-hydroxy-3-methylbutoxy)-3-(6-(methyl(2,2,6,6-tetramethylpiperidin-4-yl)amino)pyridazin-3-yl)naphthalen-2-ol; 2-(6-(methyl(2,2,6,6-tetramethylpiperidin-4-yl)amino)pyridazin-3-yl)-5-(1H-pyrazol-4-yl)benzene-1,3-diol; 3-methoxy-2-(6-(methyl(2,2,6,6-tetramethylpiperidin-4-yl)amino)pyridazin-3-yl)-5-(1H-pyrazol-4-yl)phenol; 5-(1H-pyrazol-4-yl)-2-(6-((2,2,6,6-tetramethylpiperidin-4-yl)amino)pyridazin-3-yl)-3-(trifluoromethoxy)phenol; 2-(6-(methyl(2,2,6,6-tetramethylpiperidin-4-yl)amino)pyridazin-3-yl)-5-(1-methyl-1H-pyrazol-4-yl)-3-(trifluoromethoxy)phenol; 2-(6-(methyl(2,2,6,6-tetramethylpiperidin-4-yl)amino)pyridazin-3-yl)-5-(1H-pyrazol-4-yl)-3-(trifluoromethoxy)phenol; 4-(3-hydroxy-4-(6-(methyl(2,2,6,6-tetramethylpiperidin-4-yl)amino)pyridazin-3-yl)-5-(trifluoromethoxy)phenyl)-1-methylpyridin-2(1H)-one; 3-methoxy-2-(6-(methyl(2,2,6,6-tetramethylpiperidin-4-yl)amino)pyridazin-3-yl)-5-(1-methyl-1H-pyrazol-4-yl)phenol; 3-methoxy-2-(6-(methyl(2,2,6,6-tetramethylpiperidin-4-yl)amino)pyridazin-3-yl)-5-(5,6,7,8-tetrahydroimidazo[1,2-a]pyridin-3-yl)phenol; 3-methoxy-2-(6-(methyl(2,2,6,6-tetramethylpiperidin-4-yl)amino)pyridazin-3-yl)-5-(pyridin-3-yl)phenol; 5-(1-cyclopentyl-1H-pyrazol-4-yl)-3-methoxy-2-(6-(methyl(2,2,6,6-tetramethylpiperidin-4-yl)amino)pyridazin-3-yl)phenol; 3′,5-dimethoxy-4-(6-(methyl(2,2,6,6-tetramethylpiperidin-4-yl)amino)pyridazin-3-yl)-[1,1′-biphenyl]-3-ol; 3-(benzyloxy)-2-(6-(methyl(2,2,6,6-tetramethylpiperidin-4-yl)amino)pyridazin-3-yl)-5-(5-methyloxazol-2-yl)phenol; 3-ethoxy-2-(6-(methyl(2,2,6,6-tetramethylpiperidin-4-yl)amino)pyridazin-3-yl)-5-(5-methyloxazol-2-yl)phenol; 3-(cyclopropylmethoxy)-2-(6-(methyl(2,2,6,6-tetramethylpiperidin-4-yl)amino)-pyridazin-3-yl)-5-(5-methyloxazol-2-yl)phenol; 2-methyl-5-(6-(methyl(2,2,6,6-tetramethylpiperidin-4-yl)amino)pyridazin-3-yl)-1H-benzo[d]imidazol-6-ol; 5-chloro-2-(6-(methyl(2,2,6,6-tetramethylpiperidin-4-yl)amino)pyridazin-3-yl)phenol; 5-(1H-pyrazol-1-yl)-2-(6-((2,2,6,6-tetramethylpiperidin-4-yl)amino)pyridazin-3-yl)phenol; 3-hydroxy-4-(6-((2,2,6,6-tetramethylpiperidin-4-yl)amino)pyridazin-3-yl)benzonitrile; 2-(6-((2,2-dimethylpiperidin-4-yl)oxy)pyridazin-3-yl)-5-(1H-pyrazol-1-yl)phenol; 2-(6-(methyl(2,2,6,6-tetramethylpiperidin-4-yl)amino)pyridazin-3-yl)-4-(1H-pyrazol-4-yl)phenol; 2-(6-(methyl(2,2,6,6-tetramethylpiperidin-4-yl)amino)pyridazin-3-yl)-4-(4,5,6,7-tetrahydropyrazolo[1,5-a]pyridin-3-yl)phenol; 2-(6-(methyl(2,2,6,6-tetramethylpiperidin-4-yl)amino)pyridazin-3-yl)-4-(4,5,6,7-tetrahydropyrazolo[1,5-a]pyrazin-3-yl)phenol; 4-(1H-indol-2-yl)-2-(6-(methyl(2,2,6,6-tetramethylpiperidin-4-yl)amino)pyridazin-3-yl)phenol; 4-(cyclopent-1-en-1-yl)-2-(6-(methyl(2,2,6,6-tetramethylpiperidin-4-yl)amino)pyridazin-3-yl)phenol; 2-(6-(methyl(2,2,6,6-tetramethylpiperidin-4-yl)amino)pyridazin-3-yl)-4-(1H-pyrazol-3-yl)phenol; 4-(4-hydroxy-3-(6-(methyl(2,2,6,6-tetramethylpiperidin-4-yl)amino)pyridazin-3-yl)phenyl)pyridin-2-ol; 4-(4-hydroxy-3-(6-((2,2,6,6-tetramethylpiperidin-4-yl)oxy)pyridazin-3-yl)phenyl)-1-methylpyridin-2(1H)-one; 4-(4-hydroxy-3-(6-((2,2,6,6-tetramethylpiperidin-4-yl)oxy)pyridazin-3-yl)phenyl)pyridin-2-ol; 5-(1H-indazol-7-yl)-2-(6-(methyl(2,2,6,6-tetramethylpiperidin-4-yl)amino)pyridazin-3-yl)phenol; 4-chloro-2-(6-(methyl(2,2,6,6-tetramethylpiperidin-4-yl)amino)pyridazin-3-yl)-5-(1H-pyrazol-4-yl)phenol; 4-fluoro-2-(6-(methyl(2,2,6,6-tetramethylpiperidin-4-yl)amino)pyridazin-3-yl)-5-(1H-pyrazol-4-yl)phenol; 5-fluoro-4-(1H-imidazol-4-yl)-2-(6-(methyl(2,2,6,6-tetramethylpiperidin-4-yl)amino)pyridazin-3-yl)phenol; 5-fluoro-2-(6-(methyl(2,2,6,6-tetramethylpiperidin-4-yl)amino)pyridazin-3-yl)-4-(1H-pyrazol-4-yl)phenol; 5-fluoro-2-(6-(methyl(2,2,6,6-tetramethylpiperidin-4-yl)amino)pyridazin-3-yl)-4-(1H-pyrazol-5-yl)phenol; 6-hydroxy-5-(6-(methyl(2,2,6,6-tetramethylpiperidin-4-yl)amino)pyridazin-3-yl)-2,3-dihydro-1H-inden-1-one; 6-(6-(methyl(2,2,6,6-tetramethylpiperidin-4-yl)amino)pyridazin-3-yl)-1,4-dihydroindeno[1,2-c]pyrazol-7-ol; 6-hydroxy-5-(6-(methyl(2,2,6,6-tetramethylpiperidin-4-yl)amino)pyridazin-3-yl)-2,3-dihydro-1H-inden-1-one oxime hydrochloride salt; 5-(6-(methyl(2,2,6,6-tetramethylpiperidin-4-yl)amino)pyridazin-3-yl)-2,3-dihydro-1H-indene-1,6-diol; 2-amino-6-(6-(methyl(2,2,6,6-tetramethylpiperidin-4-yl)amino)pyridazin-3-yl)-8H-indeno[1,2-d]thiazol-5-ol hydrochloride salt; 9-(6-(methyl(2,2,6,6-tetramethylpiperidin-4-yl)amino)pyridazin-3-yl)-5,6-dihydroimidazo[5,1-a]isoquinolin-8-ol hydrochloride salt; 4-hydroxy-3-(6-(methyl(2,2,6,6-tetramethylpiperidin-4-yl)amino)pyridazin-3-yl)-N-((1-methyl-TH-pyrazol-4-yl)methyl)benzamide; 4-(4-(hydroxymethyl)-1H-pyrazol-1-yl)-2-(6-(methyl(2,2,6,6-tetramethylpiperidin-4-yl)amino)pyridazin-3-yl)phenol; 5-(1H-pyrazol-4-yl)-2-(6-((2,2,6,6-tetramethylpiperidin-4-yl)methyl)pyridazin-3-yl)phenol; 6-(3-(benzyloxy)isoquinolin-6-yl)-N-methyl-N-(2,2,6,6-tetramethylpiperidin-4-yl)pyridazin-3-amine; 6-(1-(benzyloxy)isoquinolin-7-yl)-N-methyl-N-(2,2,6,6-tetramethylpiperidin-4-yl)pyridazin-3-amine; 3-fluoro-5-(2-methoxypyridin-4-yl)-2-(6-(methyl(2,2,6,6-tetramethylpiperidin-4-yl)amino)pyridazin-3-yl)phenol hydrochloride salt; 4-(3-fluoro-5-hydroxy-4-(6-(methyl(2,2,6,6-tetramethylpiperidin-4-yl)amino)pyridazin-3-yl)phenyl)pyridin-2(1H)-one hydrochloride salt; 4-(3-fluoro-5-hydroxy-4-(6-(methyl(2,2,6,6-tetramethylpiperidin-4-yl)amino)pyridazin-3-yl)phenyl)-1-methylpyridin-2(1H)-one hydrochloride salt; 5-(3-fluoro-5-hydroxy-4-(6-(methyl(2,2,6,6-tetramethylpiperidin-4-yl)amino)pyridazin-3-yl)phenyl)-1-methylpyridin-2(1H)-one hydrochloride salt; 3-fluoro-5-(1H-pyrazol-4-yl)-2-(6-((2,2,6,6-tetramethylpiperidin-4-yl)oxy)pyridazin-3-yl)phenol hydrochloride salt; 5-chloro-3-fluoro-2-(6-(methyl(2,2,6,6-tetramethylpiperidin-4-yl)amino)pyridazin-3-yl)phenol hydrochloride salt; 3-fluoro-2-(6-(methyl(2,2,6,6-tetramethylpiperidin-4-yl)amino)pyridazin-3-yl)-5-(1H-pyrazol-4-yl)phenol hydrochloride salt; 3-fluoro-2-(6-(methyl(2,2,6,6-tetramethylpiperidin-4-yl)amino)pyridazin-3-yl)-5-(1-methyl-1H-pyrazol-4-yl)phenol hydrochloride salt; 5-(5-methoxypyridin-3-yl)-2-(6-(methyl(2,2,6,6-tetramethylpiperidin-4-yl)amino)pyridazin-3-yl)phenol; 5-(3-hydroxy-4-(6-methyl(2,2,6,6-tetramethylpiperidin-4-yl)amino)pyridazin-3-yl)phenyl)pyridin-2-ol; 4-(3-hydroxy-4-(6-methyl(2,2,6,6-tetramethylpiperidin-4-yl)amino)pyridazin-3-yl)phenyl)pyridin-2-ol; 5-(6-methoxypyridin-3-yl)-2-(6-(methyl(2,2,6,6-tetramethylpiperidin-4-yl)amino)pyridazin-3-yl)phenol; 5-(3-hydroxy-4-(6-(methyl(2,2,6,6-tetramethylpiperidin-4-yl)amino)pyridazin-3-yl)phenyl)-3-(trifluoromethyl)pyridin-2-ol; 5-(3-hydroxy-4-(6-(methyl(2,2,6,6-tetramethylpiperidin-4-yl)amino)pyridazin-3-yl)phenyl)-1-methylpyridin-2(1H)-one; 4-(3-hydroxy-4-(6-(methyl(2,2,6,6-tetramethylpiperidin-4-yl)amino)pyridazin-3-yl)phenyl)-1-methylpyridin-2(1H)-one; 5-(2-methoxypyridin-4-yl)-2-(6-(methyl(2,2,6,6-tetramethylpiperidin-4-yl)amino)pyridazin-3-yl)phenol; 4-(3-hydroxy-4-(6-((2,2,6,6-tetramethylpiperidin-4-yl)oxy)pyridazin-3-yl)phenyl)pyridin-2-ol; 5-(6-(dimethylamino)pyridin-3-yl)-2-(6-(methyl(2,2,6,6-tetramethylpiperidin-4-yl)amino)pyridazin-3-yl)phenol; 4-(3-hydroxy-4-(6-((2,2,6,6-tetramethylpiperidin-4-yl)oxy)pyridazin-3-yl)phenyl)-1-methylpyridin-2(1H)-one; 2-(6-(methyl(2,2,6,6-tetramethylpiperidin-4-yl)amino)pyridazin-3-yl)-5-(pyrimidin-5-yl)phenol; 5-(3-hydroxy-4-(6-(methyl(2,2,6,6-tetramethylpiperidin-4-yl)amino)pyridazin-3-yl)phenyl)pyridin-3-ol; 1-cyclopropyl-4-(3-hydroxy-4-(6-(methyl(2,2,6,6-tetramethylpiperidin-4-yl)amino)pyridazin-3-yl)phenyl)pyridin-2(1H)-one; 2-(6-(methyl(2,2,6,6-tetramethylpiperidin-4-yl)amino)pyridazin-3-yl)-5-(1,2,3,6-tetrahydropyridin-4-yl)phenol; 5-(cyclopent-1-en-1-yl)-2-(6-(methyl(2,2,6,6-tetramethylpiperidin-4-yl)amino)pyridazin-3-yl)phenol; 5-(3,6-dihydro-2H-pyran-4-yl)-2-(6-(methyl(2,2,6,6-tetramethylpiperidin-4-yl)amino)pyridazin-3-yl)phenol; 5-(imidazo[1,5-a]pyridin-7-yl)-2-(6-(methyl(2,2,6,6-tetramethylpiperidin-4-yl)amino)pyridazin-3-yl)phenol; 5-(imidazo[1,2-a]pyridin-7-yl)-2-(6-(methyl(2,2,6,6-tetramethylpiperidin-4-yl)amino)pyridazin-3-yl)phenol; 2-(6-(methyl(2,2,6,6-tetramethylpiperidin-4-yl)amino)pyridazin-3-yl)-5-(2-methylpyridin-4-yl)phenol; 5-(1H-imidazol-2-yl)-2-(6-(methyl(2,2,6,6-tetramethylpiperidin-4-yl)amino)pyridazin-3-yl)phenol; 5-(1H-imidazol-4-yl)-2-(6-(methyl(2,2,6,6-tetramethylpiperidin-4-yl)amino)pyridazin-3-yl)phenol; 5-(imidazo[1,2-a]pyrazin-3-yl)-2-(6-(methyl(2,2,6,6-tetramethylpiperidin-4-yl)amino)pyridazin-3-yl)phenol; 2-(6-(methyl(2,2,6,6-tetramethylpiperidin-4-yl)amino)pyridazin-3-yl)-5-(5,6,7,8-tetrahydroimidazo[1,2-a]pyrazin-3-yl)phenol; 2-(6-(methyl(2,2,6,6-tetramethylpiperidin-4-yl)amino)pyridazin-3-yl)-5-(4-methyl-1H-imidazol-2-yl)phenol; 2-(6-(methyl(2,2,6,6-tetramethylpiperidin-4-yl)amino)pyridazin-3-yl)-5-(1-methyl-1H-imidazol-4-yl)phenol; 2-(6-(methyl(2,2,6,6-tetramethylpiperidin-4-yl)amino)pyridazin-3-yl)-5-(1-methyl-1H-imidazol-5-yl)phenol; 2-(6-(methyl(2,2,6,6-tetramethylpiperidin-4-yl)amino)pyridazin-3-yl)-5-(4-nitro-1H-imidazol-2-yl)phenol; 2-(6-(methyl(2,2,6,6-tetramethylpiperidin-4-yl)amino)pyridazin-3-yl)-5-(2-methyl-1H-imidazol-4-yl)phenol; 5-(1,2-dimethyl-1H-imidazol-4-yl)-2-(6-(methyl(2,2,6,6-tetramethylpiperidin-4-yl)amino)pyridazin-3-yl)phenol; 1-(3-hydroxy-4-(6-(methyl(2,2,6,6-tetramethylpiperidin-4-yl)amino)pyridazin-3-yl)phenyl)-1H-pyrazole-4-carboxamide; 2-(6-((3aR,6aS)-5-(2-hydroxyethyl)hexahydropyrrolo[3,4-c]pyrrol-2(1H)-yl)pyridazin-3-yl)-5-(1H-pyrazol-4-yl)phenol; 2-(6-((3aR,6aS)-hexahydropyrrolo[3,4-c]pyrrol-2(1H)-yl)pyridazin-3-yl)-5-(1H-pyrazol-4-yl)phenol; 2-(6-((3aR,6aS)-5-methylhexahydropyrrolo[3,4-c]pyrrol-2(1H)-yl)pyridazin-3-yl)-5-(1H-pyrazol-4-yl)phenol; 4-(3-hydroxy-4-(6-(5-methylhexahydropyrrolo[3,4-c]pyrrol-2(1H)-yl)pyridazin-3-yl)phenyl)-1-methylpyridin-2(1H)-one; 4-(3-hydroxy-4-(6-((3aR,6aR)-1-methylhexahydropyrrolo[3,4-b]pyrrol-5(1H)-yl)pyridazin-3-yl)phenyl)-1-methylpyridin-2(1H)-one; 2-(6-(2,7-diazaspiro[4.5]decan-2-yl)pyridazin-3-yl)-5-(1H-pyrazol-4-yl)phenol; and 4-(4-(6-(2,7-diazaspiro[4.5]decan-2-yl)pyridazin-3-yl)-3-hydroxyphenyl)-1-methylpyridin-2(1H)-one.

An additional representative splice modifier is RG7916 (Roche/PTC/SMAF,³⁵ 7-(4,7-diazaspiro[2.5]octan-7-yl)-2-(2,8-dimethylimidazo[1,2-b]pyridazin-6-yl)-4H-pyrido[1,2-a]pyrimidin-4-one) having the following structure:

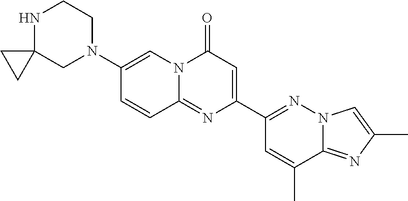 [SMILES: Cc1cn2nc(-c3cc(=O)n4cc(N5CCNC6(CC6)C5)ccc4n3)cc(C)c2n1]

An additional representative splice modifier is RG7800 (Roche) having the following structure:

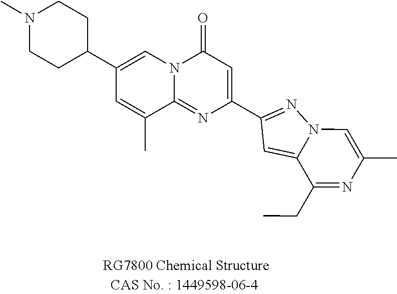

Analogues of splice modifiers such as RG7916 and RG7800 that can be used also are included, for example, 2-(2-methylimidazo[1,2-b]pyridazin-6-yl)-7-(4-methylpiperazin-1-yl)pyrido[1,2-a]pyrimidin-4-one; 7-[(8aR)-3,4,6,7,8,8a-hexahydro-1H-pyrrolo[1,2-a]pyrazin-2-yl]-2-(2-methylimidazo[1,2-b]pyridazin-6-yl)pyrido[1,2-a]pyrimidin-4-one; 7-[(8aS)-3,4,6,7,8,8a-hexahydro-1H-pyrrolo[1,2-a]pyrazin-2-yl]-2-(2,8-dimethylimidazo[1,2-b]pyridazin-6-yl)pyrido[1,2-a]pyrimidin-4-one; 7-[(8aR)-3,4,6,7,8,8a-hexahydro-TH-pyrrolo[1,2-a]pyrazin-2-yl]-2-(2,8-dimethylimidazo[1,2-b]pyridazin-6-yl)pyrido[1,2-a]pyrimidin-4-one; 7-[(8aS)-8a-methyl-1,3,4,6,7,8-hexahydropyrrolo[1,2-a]pyrazin-2-yl]-2-(2,8-dimethylimidazo[1,2-b]pyridazin-6-yl)pyrido[1,2-a]pyrimidin-4-one; 7-[(8aR)-8a-methyl-1,3,4,6,7,8-hexahydropyrrolo[1,2-a]pyrazin-2-yl]-2-(2,8-dimethylimidazo[1,2-b]pyridazin-6-yl)pyrido[1,2-a]pyrimidin-4-one; 2-(2,8-dimethylimidazo[1,2-b]pyridazin-6-yl)-7-[(3S,5R)-3,5-dimethylpiperazin-1-yl]pyrido[1,2-a]pyrimidin-4-one; 2-(2,8-dimethylimidazo[1,2-b]pyridazin-6-yl)-7-[(3S)-3-methylpiperazin-1-yl]pyrido[1,2-a]pyrimidin-4-one; 2-(2,8-dimethylimidazo[1,2-b]pyridazin-6-yl)-7-[(3R)-3-methylpiperazin-1-yl]pyrido[1,2-a]pyrimidin-4-one; 7-(1,4-diazepan-1-yl)-2-(2,8-dimethylimidazo[1,2-b]pyridazin-6-yl)pyrido[1,2-a]pyrimidin-4-one; 2-(2-methylimidazo[1,2-b]pyridazin-6-yl)-7-[(3S)-3-methylpiperazin-1-yl]pyrido[1,2-a]pyrimidin-4-one; 2-(2-methylimidazo[1,2-b]pyridazin-6-yl)-7-[(3R)-3-methylpiperazin-1-yl]pyrido[1,2-a]pyrimidin-4-one; 7-(1,4-diazepan-1-yl)-2-(2-methylimidazo[1,2-b]pyridazin-6-yl)pyrido[1,2-a]pyrimidin-4-one; 7-[(3R,5S)-3,5-dimethylpiperazin-1-yl]-2-(2-methylimidazo[1,2-b]pyridazin-6-yl)pyrido[1,2-a]pyrimidin-4-one; 7-[(8aS)-3,4,6,7,8,8a-hexahydro-TH-pyrrolo[1,2-a]pyrazin-2-yl]-2-(2-methylimidazo[1,2-b]pyridazin-6-yl)pyrido[1,2-a]pyrimidin-4-one; 7-[(8aS)-8a-methyl-1,3,4,6,7,8-hexahydropyrrolo[1,2-a]pyrazin-2-yl]-2-(2-methylimidazo[1,2-b]pyridazin-6-yl)pyrido[1,2-a]pyrimidin-4-one; 7-[(8aR)-8a-methyl-1,3,4,6,7,8-hexahydropyrrolo[1,2-a]pyrazin-2-yl]-2-(2-methylimidazo[1,2-b]pyridazin-6-yl)pyrido[1,2-a]pyrimidin-4-one; 2-(2,8-dimethylimidazo[1,2-b]pyridazin-6-yl)-7-[(3R)-3-pyrrolidin-1-ylpyrrolidin-1-yl]pyrido[1,2-a]pyrimidin-4-one; 7-(4,7-diazaspiro[2.5]octan-7-yl)-2-(2-methylimidazo[1,2-b]pyridazin-6-yl)pyrido[1,2-a]pyrimidin-4-one; 7-(4,7-diazaspiro[2.5]octan-7-yl)-2-(2,8-dimethylimidazo[1,2-b]pyridazin-6-yl)pyrido[1,2-a]pyrimidin-4-one; 2-(2-methylimidazo[1,2-b]pyridazin-6-yl)-7-[(3R)-3-pyrrolidin-1-ylpyrrolidin-1-yl]pyrido[1,2-a]pyrimidin-4-one; 2-(2,8-dimethylimidazo[1,2-b]pyridazin-6-yl)-7-(3,3-dimethylpiperazin-1-yl)pyrido[1,2-a]pyrimidin-4-one; 7-(3,3-dimethylpiperazin-1-yl)-2-(2-methylimidazo[1,2-b]pyridazin-6-yl)pyrido[1,2-a]pyrimidin-4-one; 2-(2,8-dimethylimidazo[1,2-b]pyridazin-6-yl)-9-methyl-7-[(3S)-3-methylpiperazin-1-yl]pyrido[1,2-a]pyrimidin-4-one; 2-(2,8-dimethylimidazo[1,2-b]pyridazin-6-yl)-9-methyl-7-[(3R)-3-methylpiperazin-1-yl]pyrido[1,2-a]pyrimidin-4-one; 2-(2,8-dimethylimidazo[1,2-b]pyridazin-6-yl)-7-[(3R,5S)-3,5-dimethylpiperazin-1-yl]-9-methyl-pyrido[1,2-a]pyrimidin-4-one; 2-(2,8-dimethylimidazo[1,2-b]pyridazin-6-yl)-7-(3,3-dimethylpiperazin-1-yl)-9-methyl-pyrido[1,2-a]pyrimidin-4-one; 7-(4,7-diazaspiro[2.5]octan-7-yl)-2-(2,8-dimethylimidazo[1,2-b]pyridazin-6-yl)-9-methyl-pyrido[1,2-a]pyrimidin-4-one; 2-(2,8-dimethylimidazo[1,2-b]pyridazin-6-yl)-7-[(3S,5S)-3,5-dimethylpiperazin-1-yl]pyrido[1,2-a]pyrimidin-4-one; 2-(2,8-dimethylimidazo[1,2-b]pyridazin-6-yl)-7-[(3S)-3-pyrrolidin-1-ylpyrrolidin-1-yl]pyrido[1,2-a]pyrimidin-4-one; 2-(2-methylimidazo[1,2-b]pyridazin-6-yl)-7-[(3S)-3-pyrrolidin-1-ylpyrrolidin-1-yl]pyrido[1,2-a]pyrimidin-4-one; 7-[(3S,5S)-3,5-dimethylpiperazin-1-yl]-2-(2-methylimidazo[1,2-b]pyridazin-6-yl)pyrido[1,2-a]pyrimidin-4-one; 9-methyl-2-(2-methylimidazo[1,2-b]pyridazin-6-yl)-7-[(3S)-3-methylpiperazin-1-yl]pyrido[1,2-a]pyrimidin-4-one; 9-methyl-2-(2-methylimidazo[1,2-b]pyridazin-6-yl)-7-[(3R)-3-methylpiperazin-1-yl]pyrido[1,2-a]pyrimidin-4-one; 7-[(3R,5S)-3,5-dimethylpiperazin-1-yl]-9-methyl-2-(2-methylimidazo[1,2-b]pyridazin-6-yl)pyrido[1,2-a]pyrimidin-4-one; 7-(3,3-dimethylpiperazin-1-yl)-9-methyl-2-(2-methylimidazo[1,2-b]pyridazin-6-yl)pyrido[1,2-a]pyrimidin-4-one; 7-(4,7-diazaspiro[2.5]octan-7-yl)-9-methyl-2-(2-methylimidazo[1,2-b]pyridazin-6-yl)pyrido[1,2-a]pyrimidin-4-one; 7-[(3S,5S)-3,5-dimethylpiperazin-1-yl]-9-methyl-2-(2-methylimidazo[1,2-b]pyridazin-6-yl)pyrido[1,2-a]pyrimidin-4-one; and 7-[(3R)-3-ethylpiperazin-1-yl]-2-(2-methylimidazo[1,2-b]pyridazin-6-yl)pyrido[1,2-a]pyrimidin-4-one; 7-[(8aS)-3,4,6,7,8,8a-hexahydro-1H-pyrrolo[1,2-a]pyrazin-2-yl]-2-(2,8-dimethylimidazo[1,2-b]pyridazin-6-yl)pyrido[1,2-a]pyrimidin-4-one; 7-[(8aR)-3,4,6,7,8,8a-hexahydro-TH-pyrrolo[1,2-a]pyrazin-2-yl]-2-(2,8-dimethylimidazo[1,2-b]pyridazin-6-yl)pyrido[1,2-a]pyrimidin-4-one; 2-(2,8-dimethylimidazo[1,2-b]pyridazin-6-yl)-7-[(3S,5R)-3,5-dimethylpiperazin-1-yl]pyrido[1,2-a]pyrimidin-4-one; 7-[(3R,5S)-3,5-dimethylpiperazin-1-yl]-2-(2-methylimidazo[1,2-b]pyridazin-6-yl)pyrido[1,2-a]pyrimidin-4-one; 7-[(8aS)-3,4,6,7,8,8a-hexahydro-1H-pyrrolo[1,2-a]pyrazin-2-yl]-2-(2-methylimidazo[1,2-b]pyridazin-6-yl)pyrido[1,2-a]pyrimidin-4-one; 2-(2,8-dimethylimidazo[1,2-b]pyridazin-6-yl)-9-methyl-7-[(3S)-3-methylpiperazin-1-yl]pyrido[1,2-a]pyrimidin-4-one; 7-fluoro-2-(2-methylimidazo[1,2-b]pyridazin-6-yl)pyrido[1,2-a]pyrimidin-4-one; 2-(2,8-dimethylimidazo[1,2-b]pyridazin-6-yl)-7-fluoro-pyrido[1,2-a]pyrimidin-4-one; 7-fluoro-9-methyl-2-(2-methylimidazo[1,2-b]pyridazin-6-yl)pyrido[1,2-a]pyrimidin-4-one; 2-(2,8-dimethylimidazo[1,2-b]pyridazin-6-yl)-7-fluoro-9-methyl-pyrido[1,2-a]pyrimidin-4-one; or a pharmaceutically acceptable salt thereof.

Another representative family of splice modifiers are the compounds (sudemycins) disclosed in U.S. Pat. No. 9,682,993, including (5′,Z)-5-(((1R,4R)-4-((2JE′,4JE)-5-((3R,55′)-7,7-dimethyl-1,6-dioxaspiro[2.5]octan-5-yl)-3-methylpenta-2,4-dien-1-yl)cyclohexyl)amino)-5-oxopent-3-en-2-yl methylcarbamate and (5′,Z)-5-(((1R,4R)-4-((2JE′,4JE)-5-((3R,55′)-7,7-dimethyl-1,6-dioxaspiro[2.5]octan-5-yl)-3-methylpenta-2,4-dien-1-yl)cyclohexyl)amino)-5-oxopent-3-en-2-yl dimethylcarbamate.

Yet another representative family of splice modifiers are the pladienolide compounds, including those disclosed in the following patent applications: WO 2002/060890; WO 2004/01 1459; WO 2004/01 1661; WO 2004/050890; WO 2005/052152; WO 2006/009276; WO 2008/126918; and WO 2015/175594, each of which are incorporated herein by reference. One example, of a pladienolide compound is (8E, 12E, 14E)-7-((4-Cycloheptylpiperazin-1-yl)carbonyl)oxy-3,6,16,21-tetrahydroxy-6,10,12,16,20-pentamethyl-18,19-epoxytricosa-8,12,14-trien-11-olide, also known as E7107, which is a semisynthetic derivative of the natural product pladienolide D.

### III. METHODS OF ADMINISTRATION

Any suitable cell or mammal can be administered or treated by a method or use described herein. Typically, a mammal is in need of a method described herein, that is suspected of having or expressing an abnormal or aberrant protein that is associated with a disease state.

Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In certain embodiments a mammal is a human. In certain embodiments a mammal is a non-rodent mammal (e.g., human, pig, goat, sheep, horse, dog, or the like). In certain embodiments a non-rodent mammal is a human. A mammal can be any age or at any stage of development (e.g., an adult, teen, child, infant, or a mammal in utero). A mammal can be male or female. In certain embodiments a mammal can be an animal disease model, for example, animal models having or expressing an abnormal or aberrant protein that is associated with a disease state or animal models with insufficient expression of a protein, which causes a disease state.

Mammals (subjects) treated by a method or composition described herein include adults (18 years or older) and children (less than 18 years of age). Adults include the elderly. Representative adults are 50 years or older. Children range in age from 1-2 years old, or from 2-4, 4-6, 6-18, 8-10, 10-12, 12-15 and 15-18 years old. Children also include infants. Infants typically range from 1-12 months of age.

In certain embodiments, a method includes administering a plurality of viral particles or nanoparticles to a mammal as set forth herein, where severity, frequency, progression or time of onset of one or more symptoms of a disease state, such as a neuro-degenerative disease, decreased, reduced, prevented, inhibited or delayed. In certain embodiments, a method includes administering a plurality of viral particles or nanoparticles to a mammal to treat an adverse symptom of a disease state, such as a neuro-degenerative disease. In certain embodiments, a method includes administering a plurality of viral particles or nanoparticles to a mammal to stabilize, delay or prevent worsening, or progression, or reverse and adverse symptom of a disease state, such as a neuro-degenerative disease.

In certain embodiments a method includes administering a plurality of viral particles or nanoparticles to the central nervous system, or portion thereof as set forth herein, of a mammal and severity, frequency, progression or time of onset of one or more symptoms of a disease state, such as a neuro-degenerative disease, are decreased, reduced, prevented, inhibited or delayed by at least about 5 to about 10, about 10 to about 25, about 25 to about 50, or about 50 to about 100 days.

In certain embodiments, a symptom or adverse effect comprises an early stage, middle or late stage symptom; a behavior, personality or language symptom; swallowing, movement, seizure, tremor or fidgeting symptom; ataxia; and/or a cognitive symptom such as memory, ability to organize.

In some embodiments, viral and non-viral based gene transfer methods can be used to introduce nucleic acids in mammalian cells or target tissues. Such methods can be used to administer nucleic acids encoding inhibitory RNAs, therapeutic proteins, or components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, 1992; Nabel & Feigner, 1993; Mitani & Caskey, 1993; Dillon, 1993; Miller, 1992; Van Brunt, 1988; Vigne, 1995; Kremer & Perricaudet, 1995; Haddada et al., 1995; and Yu et al., 1994.

Methods of non-viral delivery of nucleic acids include exosomes, lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in (e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91117424; WO 91116024. Delivery can be to cells (e.g. in vitro or ex vivo administration) or target tissues (e.g. in vivo administration).

In some embodiments, delivery is via the use of RNA or DNA viral based systems for the delivery of nucleic acids. Viral vectors in some aspects may be administered directly to patients (in vivo) or they can be used to treat cells in vitro or ex vivo, and then administered to patients. Viral-based systems in some embodiments include retroviral, lentivirus, adenoviral, adeno-associated and herpes simplex virus vectors for gene transfer.

A. Viral Vectors

The term “vector” refers to small carrier nucleic acid molecule, a plasmid, virus (e.g., AAV vector, retroviral vector, lentiviral vector), or other vehicle that can be manipulated by insertion or incorporation of a nucleic acid. Vectors, such as viral vectors, can be used to introduce/transfer nucleic acid sequences into cells, such that the nucleic acid sequence therein is transcribed and, if encoding a protein, subsequently translated by the cells.

An “expression vector” is a specialized vector that contains a gene or nucleic acid sequence with the necessary regulatory regions needed for expression in a host cell. An expression vector may contain at least an origin of replication for propagation in a cell and optionally additional elements, such as a heterologous nucleic acid sequence, expression control element (e.g., a promoter, enhancer), intron, ITR(s), and polyadenylation signal.

A viral vector is derived from or based upon one or more nucleic acid elements that comprise a viral genome. Exemplary viral vectors include adeno-associated virus (AAV) vectors, retroviral vectors, and lentiviral vectors.

The term “recombinant,” as a modifier of vector, such as recombinant viral, e.g., lenti- or parvo-virus (e.g., AAV) vectors, as well as a modifier of sequences such as recombinant nucleic acid sequences and polypeptides, means that the compositions have been manipulated (i.e., engineered) in a fashion that generally does not occur in nature. A particular example of a recombinant vector, such as an AAV, retroviral, or lentiviral vector would be where a nucleic acid sequence that is not normally present in the wild-type viral genome is inserted within the viral genome. An example of a recombinant nucleic acid sequence would be where a nucleic acid (e.g., gene) encodes an inhibitory RNA cloned into a vector, with or without 5′, 3′ and/or intron regions that the gene is normally associated within the viral genome. Although the term “recombinant” is not always used herein in reference to vectors, such as viral vectors, as well as sequences such as polynucleotides, “recombinant” forms including nucleic acid sequences, polynucleotides, transgenes, etc. are expressly included in spite of any such omission.

A recombinant viral “vector” is derived from the wild type genome of a virus, such as AAV, retrovirus, or lentivirus, by using molecular methods to remove the wild type genome from the virus, and replacing with a non-native nucleic acid, such as a nucleic acid sequence. Typically, for example, for AAV, one or both inverted terminal repeat (ITR) sequences of the AAV genome are retained in the recombinant AAV vector. A “recombinant” viral vector (e.g., rAAV) is distinguished from a viral (e.g., AAV) genome, since all or a part of the viral genome has been replaced with a non-native sequence with respect to the viral genomic nucleic acid such a nucleic acid encoding a transactivator or nucleic acid encoding an inhibitory RNA or nucleic acid encoding a therapeutic protein. Incorporation of such non-native nucleic acid sequences therefore defines the viral vector as a “recombinant” vector, which in the case of AAV can be referred to as a “rAAV vector.”

1. Adeno-Associated Virus

Adeno-associated virus (AAV) is a small nonpathogenic virus of the parvoviridae family. To date, numerous serologically distinct AAVs have been identified, and more than a dozen have been isolated from humans or primates. AAV is distinct from other members of this family by its dependence upon a helper virus for replication.

AAV genomes can exist in an extrachromosomal state without integrating into host cellular genomes; possess a broad host range; transduce both dividing and non-dividing cells in vitro and in vivo and maintain high levels of expression of the transduced genes. AAV viral particles are heat stable; resistant to solvents, detergents, changes in pH, and temperature; and can be column purified and/or concentrated on CsCl gradients or by other means. The AAV genome comprises a single-stranded deoxyribonucleic acid (ssDNA), either positive- or negative-sensed. The approximately 5 kb genome of AAV consists of one segment of single stranded DNA of either plus or minus polarity. The ends of the genome are short inverted terminal repeats (ITRs) that can fold into hairpin structures and serve as the origin of viral DNA replication.

An AAV “genome” refers to a recombinant nucleic acid sequence that is ultimately packaged or encapsulated to form an AAV particle. An AAV particle often comprises an AAV genome packaged with AAV capsid proteins. In cases where recombinant plasmids are used to construct or manufacture recombinant vectors, the AAV vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsulated into viral particles. Thus, an AAV vector “genome” refers to nucleic acid that is packaged or encapsulated by AAV capsid proteins.

The AAV virion (particle) is a non-enveloped, icosahedral particle approximately 25 nm in diameter. The AAV particle comprises an icosahedral symmetry comprised of three related capsid proteins, VP1, VP2 and VP3, which interact together to form the capsid. The right ORF often encodes the capsid proteins VP1, VP2, and VP3. These proteins are often found in a ratio of 1:1:10 respectively, but may be in varied ratios, and are all derived from the right-hand ORF. The VP1, VP2 and VP3 capsid proteins differ from each other by the use of alternative splicing and an unusual start codon. Deletion analysis has shown that removal or alteration of VP1 which is translated from an alternatively spliced message results in a reduced yield of infectious particles. Mutations within the VP3 coding region result in the failure to produce any single-stranded progeny DNA or infectious particles.

An AAV particle is a viral particle comprising an AAV capsid. In certain embodiments, the genome of an AAV particle encodes one, two or all VP1, VP2 and VP3 polypeptides.

The genome of most native AAVs often contain two open reading frames (ORFs), sometimes referred to as a left ORF and a right ORF. The left ORF often encodes the non-structural Rep proteins, Rep 40, Rep 52, Rep 68 and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. In certain embodiments the genome of an AAV (e.g., an rAAV) encodes some or all of the Rep proteins. In certain embodiments the genome of an AAV (e.g., an rAAV) does not encode the Rep proteins. In certain embodiments one or more of the Rep proteins can be delivered in trans and are therefore not included in an AAV particle comprising a nucleic acid encoding a polypeptide.

The ends of the AAV genome comprise short inverted terminal repeats (ITR) which have the potential to fold into T-shaped hairpin structures that serve as the origin of viral DNA replication. Accordingly, the genome of an AAV comprises one or more (e.g., a pair of) ITR sequences that flank a single stranded viral DNA genome. The ITR sequences often have a length of about 145 bases each. Within the ITR region, two elements have been described which are believed to be central to the function of the ITR, a GAGC repeat motif and the terminal resolution site (trs). The repeat motif has been shown to bind Rep when the ITR is in either a linear or hairpin conformation. This binding is thought to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

In certain embodiments, an AAV (e.g., a rAAV) comprises two ITRs. In certain embodiments, an AAV (e.g., a rAAV) comprises a pair of ITRs. In certain embodiments, an AAV (e.g., a rAAV) comprises a pair of ITRs that flank (i.e., are at each 5′ and 3′ end) of a nucleic acid sequence that at least encodes a polypeptide having function or activity.

An AAV vector (e.g., rAAV vector) can be packaged and is referred to herein as an “AAV particle” for subsequent infection (transduction) of a cell, ex vivo, in vitro or in vivo. Where a recombinant AAV vector is encapsulated or packaged into an AAV particle, the particle can also be referred to as a “rAAV particle.” In certain embodiments, an AAV particle is a rAAV particle. A rAAV particle often comprises a rAAV vector, or a portion thereof. A rAAV particle can be one or more rAAV particles (e.g., a plurality of AAV particles). rAAV particles typically comprise proteins that encapsulate or package the rAAV vector genome (e.g., capsid proteins). It is noted that reference to a rAAV vector can also be used to reference a rAAV particle.

Any suitable AAV particle (e.g., rAAV particle) can be used for a method or use herein. A rAAV particle, and/or genome comprised therein, can be derived from any suitable serotype or strain of AAV. A rAAV particle, and/or genome comprised therein, can be derived from two or more serotypes or strains of AAV. Accordingly, a rAAV can comprise proteins and/or nucleic acids, or portions thereof, of any serotype or strain of AAV, wherein the AAV particle is suitable for infection and/or transduction of a mammalian cell. Non-limiting examples of AAV serotypes include AAV1, AAV2, AAV3, AAV4, AAV5, AAV6, AAV7, AAV8, AAV9, AAV10, AAV11, AAV12, AAV-rh74, AAV-rh10 and AAV-2i8.

In certain embodiments a plurality of rAAV particles comprises particles of, or derived from, the same strain or serotype (or subgroup or variant). In certain embodiments a plurality of rAAV particles comprise a mixture of two or more different rAAV particles (e.g., of different serotypes and/or strains).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

In certain embodiments, a rAAV particle excludes certain serotypes. In one embodiment, a rAAV particle is not an AAV4 particle. In certain embodiments, a rAAV particle is antigenically or immunologically distinct from AAV4. Distinctness can be determined by standard methods. For example, ELISA and Western blots can be used to determine whether a viral particle is antigenically or immunologically distinct from AAV4. Furthermore, in certain embodiments a rAAV2 particle retains tissue tropism distinct from AAV4.

In certain embodiments, a rAAV vector based upon a first serotype genome corresponds to the serotype of one or more of the capsid proteins that package the vector. For example, the serotype of one or more AAV nucleic acids (e.g., ITRs) that comprises the AAV vector genome corresponds to the serotype of a capsid that comprises the rAAV particle.

In certain embodiments, a rAAV vector genome can be based upon an AAV (e.g., AAV2) serotype genome distinct from the serotype of one or more of the AAV capsid proteins that package the vector. For example, a rAAV vector genome can comprise AAV2 derived nucleic acids (e.g., ITRs), whereas at least one or more of the three capsid proteins are derived from a different serotype, e.g., an AAV1, AAV3, AAV4, AAV5, AAV6, AAV7, AAV8, AAV9, AAV10, AAV 11, AAV12, Rh10, Rh74 or AAV-2i8 serotype or variant thereof.

In certain embodiments, a rAAV particle or a vector genome thereof related to a reference serotype has a polynucleotide, polypeptide or subsequence thereof that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a polynucleotide, polypeptide or subsequence of an AAV1, AAV2, AAV3, AAV4, AAV5, AAV6, AAV7, AAV8, AAV9, AAV10, AAV11, AAV12, Rh10, Rh74 or AAV-2i8 particle. In particular embodiments, a rAAV particle or a vector genome thereof related to a reference serotype has a capsid or ITR sequence that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a capsid or ITR sequence of an AAV1, AAV2, AAV3, AAV4, AAV5, AAV6, AAV7, AAV8, AAV9, AAV10, AAV11, AAV12, Rh10, Rh74 or AAV-2i8 serotype.

In certain embodiments, a method herein comprises use, administration or delivery of a rAAV1, rAAV2, rAAV3, rAAV4, rAAV5, rAAV6, rAAV7, rAAV8, rAAV9, rAAV10, rAAV 11, rAAV12, rRh10, rRh74 or rAAV-2i8 particle.

In certain embodiments, a method herein comprises use, administration or delivery of a rAAV2 particle. In certain embodiments a rAAV2 particle comprises an AAV2 capsid. In certain embodiments a rAAV2 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments a rAAV2 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments, a rAAV2 particle is a variant of a native or wild-type AAV2 particle. In some aspects, one or more capsid proteins of an AAV2 variant have 1, 2, 3, 4, 5, 5-10, 10-15, 15-20 or more amino acid substitutions compared to capsid protein(s) of a native or wild-type AAV2 particle.

In certain embodiments a rAAV9 particle comprises an AAV9 capsid. In certain embodiments a rAAV9 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments a rAAV9 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments, a rAAV9 particle is a variant of a native or wild-type AAV9 particle. In some aspects, one or more capsid proteins of an AAV9 variant have 1, 2, 3, 4, 5, 5-10, 10-15, 15-20 or more amino acid substitutions compared to capsid protein(s) of a native or wild-type AAV9 particle.

In certain embodiments, a rAAV particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV1, AAV2, AAV3, AAV4, AAV5, AAV6, AAV7, AAV8, AAV9, AAV10, AAV11, AAV12, AAV-rh74, AAV-rh10 or AAV-2i8, as long as they retain one or more desired ITR functions (e.g., ability to form a hairpin, which allows DNA replication; integration of the AAV DNA into a host cell genome; and/or packaging, if desired).

In certain embodiments, a rAAV2 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

In certain embodiments, a rAAV9 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

A rAAV particle can comprise an ITR having any suitable number of “GAGC” repeats. In certain embodiments an ITR of an AAV2 particle comprises 1, 2, 3, 4, 5, 6, 7, 8, 9 or 10 or more “GAGC” repeats. In certain embodiments a rAAV2 particle comprises an ITR comprising three “GAGC” repeats. In certain embodiments a rAAV2 particle comprises an ITR which has less than four “GAGC” repeats. In certain embodiments a rAAV2 particle comprises an ITR which has more than four “GAGC” repeats. In certain embodiments an ITR of a rAAV2 particle comprises a Rep binding site wherein the fourth nucleotide in the first two “GAGC” repeats is a C rather than a T.

Exemplary suitable length of DNA can be incorporated in rAAV vectors for packaging/encapsidation into a rAAV particle can about 5 kilobases (kb) or less. In particular, embodiments, length of DNA is less than about 5 kb, less than about 4.5 kb, less than about 4 kb, less than about 3.5 kb, less than about 3 kb, or less than about 2.5 kb.

rAAV vectors that include a nucleic acid sequence that directs the expression of an RNAi or polypeptide can be generated using suitable recombinant techniques known in the art (e.g., see Sambrook et al., 1989). Recombinant AAV vectors are typically packaged into transduction-competent AAV particles and propagated using an AAV viral packaging system. A transduction-competent AAV particle is capable of binding to and entering a mammalian cell and subsequently delivering a nucleic acid cargo (e.g., a heterologous gene) to the nucleus of the cell. Thus, an intact rAAV particle that is transduction-competent is configured to transduce a mammalian cell. A rAAV particle configured to transduce a mammalian cell is often not replication competent, and requires additional protein machinery to self-replicate. Thus, a rAAV particle that is configured to transduce a mammalian cell is engineered to bind and enter a mammalian cell and deliver a nucleic acid to the cell, wherein the nucleic acid for delivery is often positioned between a pair of AAV ITRs in the rAAV genome.

Suitable host cells for producing transduction-competent AAV particles include but are not limited to microorganisms, yeast cells, insect cells, and mammalian cells that can be, or have been, used as recipients of a heterologous rAAV vectors. Cells from the stable human cell line, HEK293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments, and expresses the adenoviral E1a and E1b genes is used to generate recombinant AAV particles. The modified HEK293 cell line is readily transfected, and provides a particularly convenient platform in which to produce rAAV particles. Methods of generating high titer AAV particles capable of transducing mammalian cells are known in the art. For example, AAV particle can be made as set forth in Wright, 2008 and Wright, 2009.

In certain embodiments, AAV helper functions are introduced into the host cell by transfecting the host cell with an AAV helper construct either prior to, or concurrently with, the transfection of an AAV expression vector. AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

2. Retrovirus

Viral vectors for use as a delivered agent in the methods, compositions and uses herein include a retroviral vector (see e.g., Miller (1992) Nature, 357:455-460). Retroviral vectors are well suited for delivering nucleic acid into cells because of their ability to deliver an unrearranged, single copy gene into a broad range of rodent, primate and human somatic cells. Retroviral vectors integrate into the genome of host cells. Unlike other viral vectors, they only infect dividing cells.

Retroviruses are RNA viruses such that the viral genome is RNA. When a host cell is infected with a retrovirus, the genomic RNA is reverse transcribed into a DNA intermediate, which is integrated very efficiently into the chromosomal DNA of infected cells. This integrated DNA intermediate is referred to as a provirus. Transcription of the provirus and assembly into infectious virus occurs in the presence of an appropriate helper virus or in a cell line containing appropriate sequences permitting encapsulation without coincident production of a contaminating helper virus. A helper virus is not required for the production of the recombinant retrovirus if the sequences for encapsulation are provided by co-transfection with appropriate vectors.

The retroviral genome and the proviral DNA have three genes: the gag, the pol and the env, which are flanked by two long terminal repeat (LTR) sequences. The gag gene encodes the internal structural (matrix, capsid, and nucleocapsid) proteins and the env gene encodes viral envelope glycoproteins. The pol gene encodes products that include the RNA-directed DNA polymerase reverse transcriptase that transcribes the viral RNA into double-stranded DNA, integrase that integrate the DNA produced by reverse transcriptase into host chromosomal DNA, and protease that acts to process the encoded gag and pol genes. The 5′ and 3′ LTRs serve to promote transcription and polyadenylation of the virion RNAs. The LTR contains all other cis-acting sequences necessary for viral replication.

Retroviral vectors are described by Coffin et al., Retroviruses, Cold Spring Harbor Laboratory Press (1997). Exemplary of a retrovirus is Moloney murine leukemia virus (MMLV) or the murine stem cell virus (MSCV). Retroviral vectors can be replication-competent or replication-defective. Typically, a retroviral vector is replication-defective in which the coding regions for genes necessary for additional rounds of virion replication and packaging are deleted or replaced with other genes. Consequently, the viruses are not able to continue their typical lytic pathway once an initial target cell is infected. Such retroviral vectors, and the necessary agents to produce such viruses (e.g., packaging cell line) are commercially available (see, e.g., retroviral vectors and systems available from Clontech, such as Catalog number 634401, 631503, 631501, and others, Clontech, Mountain View, Calif.).

Such retroviral vectors can be produced as delivered agents by replacing the viral genes required for replication with the nucleic acid molecule to be delivered. The resulting genome contains an LTR at each end with the desired gene or genes in between. Methods of producing retrovirus are known to one of skill in the art (see, e.g., International published PCT Application No. WO1995/026411). The retroviral vector can be produced in a packaging cell line containing a helper plasmid or plasmids. The packaging cell line provides the viral proteins required for capsid production and the virion maturation of the vector (e.g., gag, pol and env genes). Typically, at least two separate helper plasmids (separately containing the gag and pol genes; and the env gene) are used so that recombination between the vector plasmid cannot occur. For example, the retroviral vector can be transferred into a packaging cell line using standard methods of transfection, such as calcium phosphate mediated transfection. Packaging cell lines are well known to one of skill in the art, and are commercially available. An exemplary packaging cell line is GP2-293 packaging cell line (Catalog Numbers 631505, 631507, 631512, Clontech). After sufficient time for virion product, the virus is harvested. If desired, the harvested virus can be used to infect a second packaging cell line, for example, to produce a virus with varied host tropism. The end result is a replicative incompetent recombinant retrovirus that includes the nucleic acid of interest but lacks the other structural genes such that a new virus cannot be formed in the host cell.

References illustrating the use of retroviral vectors in gene therapy include: Clowes et al., (1994) J. Clin. Invest. 93:644-651; Kiem et al., (1994) Blood 83:1467-1473; Salmons and Gunzberg (1993) Human Gene Therapy 4:129-141; Grossman and Wilson (1993) Curr. Opin. in Genetics and Devel. 3:110-114; Sheridan (2011) Nature Biotechnology, 29:121; Cassani et al. (2009) Blood, 114:3546-3556.

3. Lentivirus

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif vpr, vpu and nef are deleted making the vector biologically safe. Lentiviral vectors are well known in the art (see, e.g., U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell, wherein a suitable host cell is transfected with two or more vectors carrying the packaging functions, namely gag, pol and env, as well as rev and tat, is described in U.S. Pat. No. 5,994,136, incorporated herein by reference.

The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTRs serve to promote transcription and polyadenylation of the virion RNAs. The LTR contains all other cis-acting sequences necessary for viral replication. Lentiviruses have additional genes including vif vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

4. Other Viral Vectors

The development and utility of viral vectors for gene delivery is constantly improving and evolving. Other viral vectors such as poxvirus; e.g., vaccinia virus (Gnant et al., 1999; Gnant et al., 1999), alpha virus; e.g., sindbis virus, Semliki forest virus (Lundstrom, 1999), reovirus (Coffey et al., 1998) and influenza A virus (Neumann et al., 1999) are contemplated for use in the present disclosure and may be selected according to the requisite properties of the target system.

5. Chimeric Viral Vectors

Chimeric or hybrid viral vectors are being developed for use in therapeutic gene delivery and are contemplated for use in the present disclosure. Chimeric poxviral/retroviral vectors (Holzer et al., 1999), adenoviral/retroviral vectors (Feng et al., 1997; Bilbao et al., 1997; Caplen et al., 2000) and adenoviral/adeno-associated viral vectors (Fisher et al., 1996; U.S. Pat. No. 5,871,982) have been described. These “chimeric” viral gene transfer systems can exploit the favorable features of two or more parent viral species. For example, Wilson et al., provide a chimeric vector construct which comprises a portion of an adenovirus, AAV 5′ and 3′ ITR sequences and a selected transgene, described below (U.S. Pat. No. 5,871,983, specifically incorporate herein by reference).

B. Nanoparticles

1. Lipid-Based Nanoparticles

In some embodiments, a lipid-based nanoparticle is a liposome, an exosome, a lipid preparation, or another lipid-based nanoparticle, such as a lipid-based vesicle (e.g., a DOTAP:cholesterol vesicle). Lipid-based nanoparticles may be positively charged, negatively charged, or neutral.

a. Liposomes

A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Liposomes provided herein include unilamellar liposomes, multilamellar liposomes, and multivesicular liposomes. Liposomes provided herein may be positively charged, negatively charged, or neutrally charged. In certain embodiments, the liposomes are neutral in charge.

A multilamellar liposome has multiple lipid layers separated by aqueous medium. Such liposomes form spontaneously when lipids comprising phospholipids are suspended in an excess of aqueous solution. The lipid components undergo self-rearrangement before the formation of closed structures and entrap water and dissolved solutes between the lipid bilayers. Lipophilic molecules or molecules with lipophilic regions may also dissolve in or associate with the lipid bilayer.

In specific aspects, a polypeptide, a nucleic acid, or a small molecule drug may be, for example, encapsulated in the aqueous interior of a liposome, interspersed within the lipid bilayer of a liposome, attached to a liposome via a linking molecule that is associated with both the liposome and the polypeptide/nucleic acid, entrapped in a liposome, complexed with a liposome, or the like.

A liposome used according to the present embodiments can be made by different methods, as would be known to one of ordinary skill in the art. For example, a phospholipid, such as for example the neutral phospholipid dioleoylphosphatidylcholine (DOPC), is dissolved in tert-butanol. The lipid(s) is then mixed with a polypeptide, nucleic acid, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min to 2 h, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

The dried lipids or lyophilized liposomes prepared as described above may be dehydrated and reconstituted in a solution of a protein or peptide and diluted to an appropriate concentration with a suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

Additional liposomes which may be useful with the present embodiments include cationic liposomes, for example, as described in WO02/100435A1, U.S. Pat. No. 5,962,016, U.S. Application 2004/0208921, WO03/015757A1, WO04029213A2, U.S. Pat. Nos. 5,030,453, and 6,680,068, all of which are hereby incorporated by reference in their entirety without disclaimer.

In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040, each incorporated herein by reference.

In certain embodiments, the lipid-based nanoparticle is a neutral liposome (e.g., a DOPC liposome). “Neutral liposomes” or “non-charged liposomes”, as used herein, are defined as liposomes having one or more lipid components that yield an essentially-neutral, net charge (substantially non-charged). By “essentially neutral” or “essentially non-charged”, it is meant that few, if any, lipid components within a given population (e.g., a population of liposomes) include a charge that is not canceled by an opposite charge of another component (i.e., fewer than 10% of components include a non-canceled charge, more preferably fewer than 5%, and most preferably fewer than 1%). In certain embodiments, neutral liposomes may include mostly lipids and/or phospholipids that are themselves neutral under physiological conditions (i.e., at about pH 7).

Liposomes and/or lipid-based nanoparticles of the present embodiments may comprise a phospholipid. In certain embodiments, a single kind of phospholipid may be used in the creation of liposomes (e.g., a neutral phospholipid, such as DOPC, may be used to generate neutral liposomes). In other embodiments, more than one kind of phospholipid may be used to create liposomes. Phospholipids may be from natural or synthetic sources. Phospholipids include, for example, phosphatidylcholines, phosphatidylglycerols, and phosphatidylethanolamines; because phosphatidylethanolamines and phosphatidyl cholines are non-charged under physiological conditions (i.e., at about pH 7), these compounds may be particularly useful for generating neutral liposomes. In certain embodiments, the phospholipid DOPC is used to produce non-charged liposomes. In certain embodiments, a lipid that is not a phospholipid (e.g., a cholesterol) may be used

Phospholipids include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

b. Exosomes

“Extracellular vesicles” and “EVs” are cell-derived and cell-secreted microvesicles which, as a class, include exosomes, exosome-like vesicles, ectosomes (which result from budding of vesicles directly from the plasma membrane), microparticles, microvesicles, shedding microvesicles (SMVs), nanoparticles and even (large) apoptotic blebs or bodies (resulting from cell death) or membrane particles.

The terms “microvesicle” and “exosomes,” as used herein, refer to a membranous particle having a diameter (or largest dimension where the particles is not spheroid) of between about 10 nm to about 5000 nm, more typically between 30 nm and 1000 nm, and most typically between about 50 nm and 750 nm, wherein at least part of the membrane of the exosomes is directly obtained from a cell. Most commonly, exosomes will have a size (average diameter) that is up to 5% of the size of the donor cell. Therefore, especially contemplated exosomes include those that are shed from a cell.

Exosomes may be detected in or isolated from any suitable sample type, such as, for example, body fluids. As used herein, the term “isolated” refers to separation out of its natural environment and is meant to include at least partial purification and may include substantial purification. As used herein, the term “sample” refers to any sample suitable for the methods provided by the present invention. The sample may be any sample that includes exosomes suitable for detection or isolation. Sources of samples include blood, bone marrow, pleural fluid, peritoneal fluid, cerebrospinal fluid, urine, saliva, amniotic fluid, malignant ascites, broncho-alveolar lavage fluid, synovial fluid, breast milk, sweat, tears, joint fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer.

Exosomes may also be isolated from tissue samples, such as surgical samples, biopsy samples, tissues, feces, and cultured cells. When isolating exosomes from tissue sources it may be necessary to homogenize the tissue in order to obtain a single cell suspension followed by lysis of the cells to release the exosomes. When isolating exosomes from tissue samples it is important to select homogenization and lysis procedures that do not result in disruption of the exosomes. Exosomes contemplated herein are preferably isolated from body fluid in a physiologically acceptable solution, for example, buffered saline, growth medium, various aqueous medium, etc.

Exosomes may be isolated from freshly collected samples or from samples that have been stored frozen or refrigerated. In some embodiments, exosomes may be isolated from cell culture medium. Although not necessary, higher purity exosomes may be obtained if fluid samples are clarified before precipitation with a volume-excluding polymer, to remove any debris from the sample. Methods of clarification include centrifugation, ultracentrifugation, filtration, or ultrafiltration. Most typically, exosomes can be isolated by numerous methods well-known in the art. One preferred method is differential centrifugation from body fluids or cell culture supernatants. Exemplary methods for isolation of exosomes are described in (Losche et al., 2004; Mesri and Altieri, 1998; Morel et al., 2004). Alternatively, exosomes may also be isolated via flow cytometry as described in (Combes et al., 1997).

One accepted protocol for isolation of exosomes includes ultracentrifugation, often in combination with sucrose density gradients or sucrose cushions to float the relatively low-density exosomes. Isolation of exosomes by sequential differential centrifugations is complicated by the possibility of overlapping size distributions with other microvesicles or macromolecular complexes. Furthermore, centrifugation may provide insufficient means to separate vesicles based on their sizes. However, sequential centrifugations, when combined with sucrose gradient ultracentrifugation, can provide high enrichment of exosomes.

Isolation of exosomes based on size, using alternatives to the ultracentrifugation routes, is another option. Successful purification of exosomes using ultrafiltration procedures that are less time consuming than ultracentrifugation, and do not require use of special equipment have been reported. Similarly, a commercial kit is available (EXOMIR™, Bioo Scientific) which allows removal of cells, platelets, and cellular debris on one microfilter and capturing of vesicles bigger than 30 nm on a second microfilter using positive pressure to drive the fluid. However, for this process, the exosomes are not recovered, their RNA content is directly extracted from the material caught on the second microfilter, which can then be used for PCR analysis. HPLC-based protocols could potentially allow one to obtain highly pure exosomes, though these processes require dedicated equipment and are difficult to scale up. A significant problem is that both blood and cell culture media contain large numbers of nanoparticles (some non-vesicular) in the same size range as exosomes. For example, some miRNAs may be contained within extracellular protein complexes rather than exosomes; however, treatment with protease (e.g., proteinase K) can be performed to eliminate any possible contamination with “extraexosomal” protein.

In another embodiment, exosomes may be captured by techniques commonly used to enrich a sample for exosomes, such as those involving immunospecific interactions (e.g., immunomagnetic capture). Immunomagnetic capture, also known as immunomagnetic cell separation, typically involves attaching antibodies directed to proteins found on a particular cell type to small paramagnetic beads. When the antibody-coated beads are mixed with a sample, such as blood, they attach to and surround the particular cell. The sample is then placed in a strong magnetic field, causing the beads to pellet to one side. After removing the blood, captured cells are retained with the beads. Many variations of this general method are well-known in the art and suitable for use to isolate exosomes. In one example, the exosomes may be attached to magnetic beads (e.g., aldehyde/sulphate beads) and then an antibody is added to the mixture to recognize an epitope on the surface of the exosomes that are attached to the beads.

As will be appreciated by one of skill in the art, prior or subsequent to loading with cargo, exosomes may be further altered by inclusion of a targeting moiety to enhance the utility thereof as a vehicle for delivery of cargo. In this regard, exosomes may be engineered to incorporate an entity that specifically targets a particular cell to tissue type. This target-specific entity, e.g., peptide having affinity for a receptor or ligand on the target cell or tissue, may be integrated within the exosomal membrane, for example, by fusion to an exosomal membrane marker using methods well-established in the art.

2. Nonlipid Nanoparticles

Spherical Nucleic Acid (SNA™) constructs and other nanoparticles (particularly gold nanoparticles) are also contemplated as a means to deliver chimeric minigenes to intended target cells. Due to their dense loading, a majority of cargo (e.g., DNA) remains bound to the constructs inside cells, conferring nucleic acid stability and resistance to enzymatic degradation. For all cell types studied (e.g., neurons, tumor cell lines, etc.) the constructs demonstrate a transfection efficiency of 99% with no need for carriers or transfection agents. The unique target binding affinity and specificity of the constructs allow exquisite specificity for matched target sequences (i.e., limited off-target effects). The constructs significantly outperform leading conventional transfection reagents (Lipofectamine 2000 and Cytofectin). The constructs can enter a variety of cultured cells, primary cells, and tissues with no apparent toxicity. The constructs elicit minimal changes in global gene expression as measured by whole-genome microarray studies and cytokine-specific protein assays. Any number of single or combinatorial agents (e.g., proteins, peptides, small molecules) can be used to tailor the surface of the constructs. See, e.g., Jensen et al., Sci. Transl. Med. 5, 209ra152 (2013).

Self-assembling nanoparticles with nucleic acid cargo may be constructed with polyethyleneimine (PEI) that is PEGylated with an Arg-Gly-Asp (RGD) peptide ligand attached at the distal end of the polyethylene glycol (PEG). Nanoplexes may be prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes (see, e.g., Bartlett et al., PNAS, 104:39, 2007).

C. Encapsulated Cell Implantation

The chimeric minigenes herein can be delivered ex vivo to cells, which are then encapsulated and implanted in order to deliver the target gene to a patient. For example, cells isolated from a patient or a donor introduced with an exogenous heterologous nucleic acid can be delivered directly to a patient by implantation of encapsulated cells. The advantage of implantation of encapsulated cells is that the immune response to the cells is reduced by the encapsulation. Thus, provided herein is a method of administering a genetically modified cell or cells to a subject. The number of cells that are delivered depends on the desired effect, the particular nucleic acid, the subject being treated and other similar factors, and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, or fetal liver. For example, the genetically modified cells can be pluripotent or totipotent stem cells (including induced pluripotent stem cells) or can be embryonic, fetal, or fully differentiated cells. The genetically modified cells can be cells from the same subject or can be cells from the same or different species as the recipient subject. In a preferred example, the cell used for gene therapy is autologous to the patient. Methods of genetically modifying cells and transplanting cells are known in the art.

Typically, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, Meth. Enzymol. (1993) 217:599-618; Cotten et al., Meth. Enzymol. (1993) 217:618-644; Cline, Pharmac. Ther. (1985) 29:69-92) and can be used provided that the necessary developmental and physiological functions of the recipient cells are not disrupted. In particular examples, the method is one that permits stable transfer of the nucleic acid to the cell, so that the nucleic acid is expressible by the cell and heritable and expressible by its cell progeny.

Encapsulation can be performed using an alginate microcapsule coated with an alginate/polylysine complex. Hydrogel microcapsules have been extensively investigated for encapsulation of living cells or cell aggregates for tissue engineering and regenerative medicine (Orive, et al. Nat. Medicine 2003, 9, 104; Paul, et al., Regen. Med. 2009, 4, 733; Read, et al. Biotechnol. 2001, 19, 29) In general, capsules are designed to allow facile diffusion of oxygen and nutrients to the encapsulated cells, while releasing the therapeutic proteins secreted by the cells, and to protect the cells from attack by the immune system. These have been developed as potential therapeutics for a range of diseases including type I diabetes, cancer, and neurodegenerative disorders such as Parkinson's (Wilson et al. Adv. Drug. Deliv. Rev. 2008, 60, 124; Joki, et al. Nat. Biotech. 2001, 19, 35; Kishima, et al. Neurobiol. Dis. 2004, 16, 428). One of the most common capsule formulations is based on alginate hydrogels, which can be formed through ionic crosslinking. In a typical process, the cells are first blended with a viscous alginate solution. The cell suspension is then processed into micro-droplets using different methods such as air shear, acoustic vibration or electrostatic droplet formation (Rabanel et al. Biotechnol. Prog. 2009, 25, 946). The alginate droplet is gelled upon contact with a solution of divalent ions, such as Ca2+ or Ba2+.

Capsules are disclosed for transplanting mammalian cells into a subject. The capsules are formed from a biocompatible, hydrogel-forming polymer encapsulating the cells to be transplanted. In order to inhibit capsular overgrowth (fibrosis), the structure of the capsules prevents cellular material from being located on the surface of the capsule. Additionally, the structure of the capsules ensures that adequate gas exchange occurs with the cells and nutrients are received by the cells encapsulated therein. Optionally, the capsules also contain one or more anti-inflammatory drugs encapsulated therein for controlled release.

The disclosed compositions are formed from a biocompatible, hydrogel-forming polymer encapsulating the cells to be transplanted. Examples of materials which can be used to form a suitable hydrogel include polysaccharides such as alginate, collagen, chitosan, sodium cellulose sulfate, gelatin and agarose, water soluble polyacrylates, polyphosphazines, poly(acrylic acids), poly(methacrylic acids), poly(alkylene oxides), poly(vinyl acetate), polyvinylpyrrolidone (PVP), and copolymers and blends of each. See, for example, U.S. Pat. Nos. 5,709,854, 6,129,761, 6,858,229, and 9,555,007.

### IV. PHARMACEUTICAL COMPOSITIONS

As used herein the term “pharmaceutically acceptable” and “physiologically acceptable” mean a biologically acceptable composition, formulation, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Such composition, “pharmaceutically acceptable” and “physiologically acceptable” formulations and compositions can be sterile. Such pharmaceutical formulations and compositions may be used, for example in administering a viral particle or nanoparticle to a subject.

Such formulations and compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Supplementary active compounds (e.g., preservatives, antibacterial, antiviral and antifungal agents) can also be incorporated into the formulations and compositions.

Pharmaceutical compositions typically contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as surfactants, wetting or emulsifying agents, pH buffering substances, and the like, may be present in such vehicles.

Pharmaceutical compositions can be formulated to be compatible with a particular route of administration or delivery, as set forth herein or known to one of skill in the art. Thus, pharmaceutical compositions include carriers, diluents, or excipients suitable for administration or delivery by various routes.

Pharmaceutical forms suitable for injection or infusion of viral particles or nanoparticles can include sterile aqueous solutions or dispersions which are adapted for the extemporaneous preparation of sterile injectable or infusible solutions or dispersions, optionally encapsulated in liposomes. In all cases, the ultimate form should be a sterile fluid and stable under the conditions of manufacture, use and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Isotonic agents, for example, sugars, buffers or salts (e.g., sodium chloride) can be included. Prolonged absorption of injectable compositions can be brought about by the use in the compositions of agents delaying absorption, for example, aluminum monostearate and gelatin.

Solutions or suspensions of viral particles or nanoparticles can optionally include one or more of the following components: a sterile diluent such as water for injection, saline solution, such as phosphate buffered saline (PBS), artificial CSF, a surfactants, fixed oils, a polyol (for example, glycerol, propylene glycol, and liquid polyethylene glycol, and the like), glycerin, or other synthetic solvents; antibacterial and antifungal agents such as parabens, chlorobutanol, phenol, ascorbic acid, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Pharmaceutical formulations, compositions and delivery systems appropriate for the compositions, methods and uses of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

Viral particles, nanoparticles, and their compositions may be formulated in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms are dependent upon the number of viral particles or nanoparticles believed necessary to produce the desired effect(s). The amount necessary can be formulated in a single dose, or can be formulated in multiple dosage units. The dose may be adjusted to a suitable viral particle or nanoparticle concentration, optionally combined with an anti-inflammatory agent, and packaged for use.

In one embodiment, pharmaceutical compositions will include sufficient genetic material to provide a therapeutically effective amount, i.e., an amount sufficient to reduce or ameliorate symptoms or an adverse effect of a disease state in question or an amount sufficient to confer the desired benefit.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Thus, for example, viral particles, nanoparticles, and pharmaceutical compositions thereof can be packaged in single or multiple unit dosage form for ease of administration and uniformity of dosage.

Formulations containing viral particles or nanoparticles typically contain an effective amount, the effective amount being readily determined by one skilled in the art. The viral particles or nanoparticles may typically range from about 1% to about 95% (w/w) of the composition, or even higher if suitable. The quantity to be administered depends upon factors such as the age, weight and physical condition of the mammal or the human subject considered for treatment. Effective dosages can be established by one of ordinary skill in the art through routine trials establishing dose response curves.

### V. DEFINITIONS

The terms “polynucleotide,” “nucleic acid” and “transgene” are used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, rRNA, tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction.

A nucleic acid encoding a polypeptide often comprises an open reading frame that encodes the polypeptide. Unless otherwise indicated, a particular nucleic acid sequence also includes degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

A “promoter” refers to a nucleotide sequence, usually upstream (5′) of a coding sequence, which directs and/or controls the expression of the coding sequence by providing the recognition for RNA polymerase and other factors required for proper transcription. “Promoter” includes a minimal promoter that is a short DNA sequence comprised of a TATA-box and optionally other sequences that serve to specify the site of transcription initiation, to which regulatory elements are added for control of expression.

An “enhancer” is a DNA sequence that can stimulate transcription activity and may be an innate element of the promoter or a heterologous element that enhances the level or tissue specificity of expression. It is capable of operating in either orientation (5′->3′ or 3′->5′), and may be capable of functioning even when positioned either upstream or downstream of the promoter.

Promoters and/or enhancers may be derived in their entirety from a native gene, or be composed of different elements derived from different elements found in nature, or even be comprised of synthetic DNA segments. A promoter or enhancer may comprise DNA sequences that are involved in the binding of protein factors that modulate/control effectiveness of transcription initiation in response to stimuli, physiological or developmental conditions.

Non-limiting examples include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

A “transgene” is used herein to conveniently refer to a nucleic acid sequence/polynucleotide that is intended or has been introduced into a cell or organism. Transgenes include any nucleic acid, such as a gene that encodes an inhibitory RNA or polypeptide or protein, and are generally heterologous with respect to naturally occurring AAV genomic sequences.

The term “transduce” refers to introduction of a nucleic acid sequence into a cell or host organism by way of a vector (e.g., a viral particle). Introduction of a transgene into a cell by a viral particle is can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, transgene transcribed and the encoded inhibitory RNA or protein expressed. For gene therapy uses and methods, a transduced cell can be in a mammal.

Transgenes under control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting a suitable promoter (constitutive versus inducible; strong versus weak), it is possible to control both the existence and level of expression of a polypeptide in the genetically modified cell. If the gene encoding the polypeptide is under the control of an inducible promoter, delivery of the polypeptide in situ is triggered by exposing the genetically modified cell in situ to conditions for permitting transcription of the polypeptide, e.g., by intraperitoneal injection of specific inducers of the inducible promoters which control transcription of the agent. For example, in situ expression by genetically modified cells of a polypeptide encoded by a gene under the control of the metallothionein promoter, is enhanced by contacting the genetically modified cells with a solution containing the appropriate (i.e., inducing) metal ions in situ.

A nucleic acid/transgene is “operably linked” when it is placed into a functional relationship with another nucleic acid sequence. A nucleic acid/transgene encoding and RNAi or a polypeptide, or a nucleic acid directing expression of a polypeptide may include an inducible promoter, or a tissue-specific promoter for controlling transcription of the encoded polypeptide. A nucleic acid operably linked to an expression control element can also be referred to as an expression cassette.

In certain embodiments, CNS-specific or inducible promoters, enhancers and the like, are employed in the methods and uses described herein. Non-limiting examples of CNS-specific promoters include those isolated from the genes from myelin basic protein (MBP), glial fibrillary acid protein (GFAP), and neuron specific enolase (NSE). Non-limiting examples of inducible promoters include DNA responsive elements for ecdysone, tetracycline, hypoxia and IFN.

In certain embodiments, an expression control element comprises a CMV enhancer. In certain embodiments, an expression control element comprises a beta actin promoter. In certain embodiments, an expression control element comprises a chicken beta actin promoter. In certain embodiments, an expression control element comprises a CMV enhancer and a chicken beta actin promoter.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a protein encoded thereby, and some codons of the nucleic acid variant will be changed which in turn changes the amino acids of a protein encoded thereby.

The terms “protein” and “polypeptide” are used interchangeably herein. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide retains some degree of function or activity. Accordingly, in methods and uses of the invention, such polypeptides encoded by nucleic acid sequences are not required to be identical to the endogenous protein that is defective, or whose activity, function, or expression is insufficient, deficient or absent in a treated mammal.

Non-limiting examples of modifications include one or more nucleotide or amino acid substitutions (e.g., about 1 to about 3, about 3 to about 5, about 5 to about 10, about 10 to about 15, about 15 to about 20, about 20 to about 25, about 25 to about 30, about 30 to about 40, about 40 to about 50, about 50 to about 100, about 100 to about 150, about 150 to about 200, about 200 to about 250, about 250 to about 500, about 500 to about 750, about 750 to about 1000 or more nucleotides or residues).

An example of an amino acid modification is a conservative amino acid substitution or a deletion. In particular embodiments, a modified or variant sequence retains at least part of a function or activity of the unmodified sequence (e.g., wild-type sequence).

Another example of an amino acid modification is a targeting peptide introduced into a capsid protein of a viral particle. Peptides have been identified that target recombinant viral vectors or nanoparticles, to the central nervous system, such as vascular endothelial cells. Thus, for example, endothelial cells lining brain blood vessels can be targeted by the modified recombinant viral particles or nanoparticles.

A recombinant virus so modified may preferentially bind to one type of tissue (e.g., CNS tissue) over another type of tissue (e.g., liver tissue). In certain embodiments, a recombinant virus bearing a modified capsid protein may “target” brain vascular epithelia tissue by binding at level higher than a comparable, unmodified capsid protein. For example, a recombinant virus having a modified capsid protein may bind to brain vascular epithelia tissue at a level 50% to 100% greater than an unmodified recombinant virus.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein. In certain embodiments, the fragment or portion is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

The terms “treat” and “treatment” refer to both therapeutic treatment and prophylactic or preventative measures, wherein the object is to prevent, inhibit, reduce, or decrease an undesired physiological change or disorder, such as the development, progression or worsening of the disorder. For purposes of this invention, beneficial or desired clinical results include, but are not limited to, alleviation of symptoms, diminishment of extent of disease, stabilizing a (i.e., not worsening or progressing) symptom or adverse effect of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those predisposed (e.g., as determined by a genetic assay).

The terms “comprising,” “having,” “including,” and “containing” are to be construed as open-ended terms (i.e., meaning “including, but not limited to”) unless otherwise noted.

All methods and uses described herein can be performed in any suitable order unless otherwise indicated herein or otherwise clearly contradicted by context. The use of any and all examples, or exemplary language (e.g., “such as” or “for example”) provided herein, is intended merely to better illuminate the invention and does not pose a limitation on the scope of the invention unless otherwise claimed. No language in the specification should be construed as indicating any non-claimed element as essential to the practice of the invention.

All of the features disclosed herein may be combined in any combination. Each feature disclosed in the specification may be replaced by an alternative feature serving a same, equivalent, or similar purpose. Thus, unless expressly stated otherwise, disclosed features (e.g., modified nucleic acid, vector, plasmid, a recombinant vector sequence, vector genome, or viral particle) are an example of a genus of equivalent or similar features.

As used herein, the forms “a”, “and,” and “the” include singular and plural referents unless the context clearly indicates otherwise. Thus, for example, reference to “a nucleic acid” includes a plurality of such nucleic acids, reference to “a vector” includes a plurality of such vectors, and reference to “a virus” or “AAV or rAAV particle” includes a plurality of such virions/AAV or rAAV particles.

The term “about” at used herein refers to a values that is within 10% (plus or minus) of a reference value.

Recitation of ranges of values herein are merely intended to serve as a shorthand method of referring individually to each separate value falling within the range, unless otherwise indicated herein, and each separate value is incorporated into the specification as if it were individually recited herein.

Accordingly, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-20, 10-50, 30-50, 50-100, 100-300, 100-1,000, 1,000-3,000, 2,000-4,000, 4,000-6,000, etc.

### VI. KITS

The invention provides kits with packaging material and one or more components therein. A kit typically includes a label or packaging insert including a description of the components or instructions for use in vitro, in vivo, or ex vivo, of the components therein. A kit can contain a collection of such components, e.g., a nucleic acid, recombinant vector, viral particles, splicing modifier molecules, and optionally a second active agent, such as another compound, agent, drug or composition.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, or an electrical storage media such as RAM and ROM or hybrids of these such as magnetic/optical storage media, FLASH memory, hybrids and memory type cards.

### VII. EXAMPLES

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed.

### Example 1

Spinal muscular atrophy (SMA) is an autosomal recessive disease caused by mutations in the SMN1 gene. The homologous SMN2 gene cannot functionally compensate for SMN1 mutations because exon 7 is skipped in the majority of SMN2 transcripts, producing an unstable protein³³. When SMN1 is absent, only 10% of SMN2 protein is produced, which corresponds to the fraction of SMN2 transcripts in which exon7 is included. Modulation of the low functioning SMN2 “back-up” gene by correcting SMN2 exon 7 skipping is one of the most successful approaches to treat SMA.

Recently, Nusinersen (Ionis), an antisense oligonucleotide (ASO) that induces SMN2 exon7 inclusion, has become the first FDA-approved drug to treat SMA³⁴. Small molecules that induce exon 7 inclusion, such as LMI070 (Spinrazar™, Novartis³¹) and RG7800 (Roche/PTC/SMAF³⁵), are in clinical development.

The structure of LMI070 (Spinraza™, Novartis³¹) is as follows:

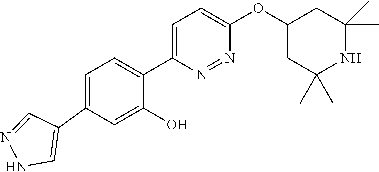 [SMILES: CC1(C)CC(Oc2ccc(-c3ccc(-c4cn[nH]c4)cc3O)nn2)CC(C)(C)N1]

The structure of RG7916 (Roche/PTC/SMAF³⁵) is as follows:

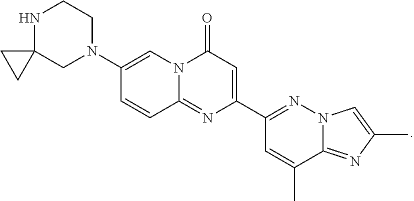 [SMILES: Cc1cn2nc(-c3cc(=O)n4cc(N5CCNC6(CC6)C5)ccc4n3)cc(C)c2n1]

In one embodiment, drug-induced SMN2 alternative splicing is used for gene expression control. In one aspect, a SMN2 minigene fused to a cDNA encoding a mammalian transactivator in which exon 7 inclusion or exclusion determines transactivator translation. The small splicing modifier molecule LMI070 can correct exon 7 skipping for transactivator expression, which will then induce transcription from an optimized target gene expression cassette.

### Example 2

A chimeric SMN2/transactivator minigene was generated in which the production of the transactivator is dependent on SMN2 exon 7 inclusion. The resulting transactivator binds an optimized promoter and activates the expression of downstream artificial miRNAs (FIG. 2). This system has: 1) minimal miRNA expression in the off state, 2) significant induction of miRNA expression by the transactivator, 3) control over transactivator levels, and subsequently miRNA expression levels, and 4) a size allowable for packaging into a single recombinant adeno-associated virus (AAV). RNAi expression control will avoid sustained co-opting of the RNAi pathway and minimize chronic unintended silencing of off-target genes.

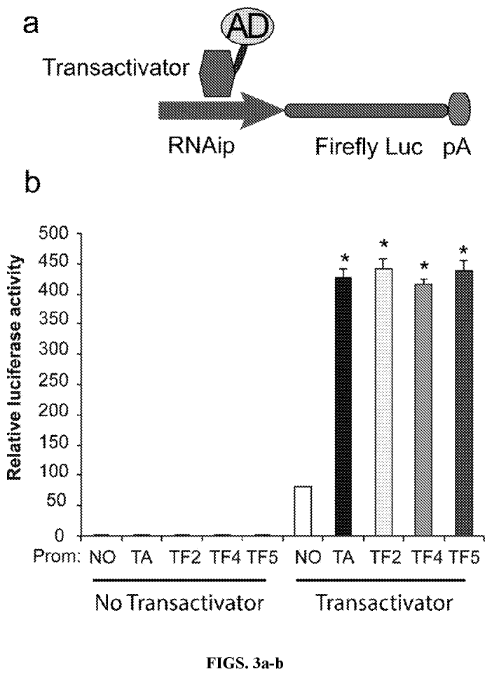

Multiple DNA binding sites for a mammalian transactivator were cloned upstream of our optimized miRNA promoters. The transactivator is a fusion protein of a mammalian zinc finger protein modified to bind a specific DNA binding sequence and the Vp16 domain from Herpes simplex virus^{36, 37}. Note that the Vp16 domain was chosen instead of the more potent activator domains (e.g., Vp64) to minimize possible gene activation as result of off-target binding of the transactivator protein in the genome. Activation of the miRNA promoters was evaluated by the luciferase ratio after triple-transfection with the transactivator, the miRNA promoter driving Firefly expression, and the Renilla luciferase expression cassettes (FIGS. 3a-b).

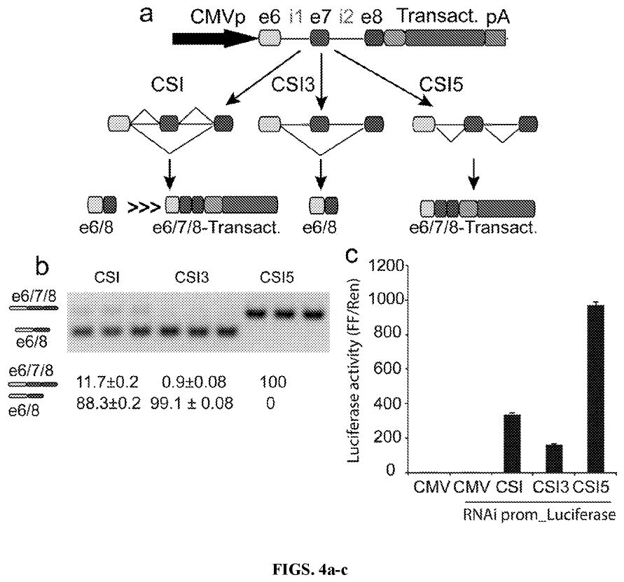

To regulate transactivator expression, the transactivator was cloned downstream of a self-cleaving 2A peptide and the SMN2 minigene comprising exons 6-7 and the 5′ end of exon 8, and minimal intronic intervening sequences necessary to recapitulate SMN2 splicing³⁸ (FIG. 4a). Note that 10% of the transcripts include exon 7, similar to the native SMN2 genetic state. The transcripts produce transactivator that partially activates the RNAi expression cassette. Thus, the 3′ and 5′ Exon7 splicing sites in the SMN2/transactivator minigene were modified to constitutively exclude (CSI3, 3′ modified) or include (CSI5, 5′ modified) exon 7, which minimizes RNAi promoter background activation³⁹ (FIGS. 4b-c). Importantly, exon 7 inclusion is LMI070 dose-responsive (FIGS. 5a-b). Also, the entire cassette fits into rAAV.

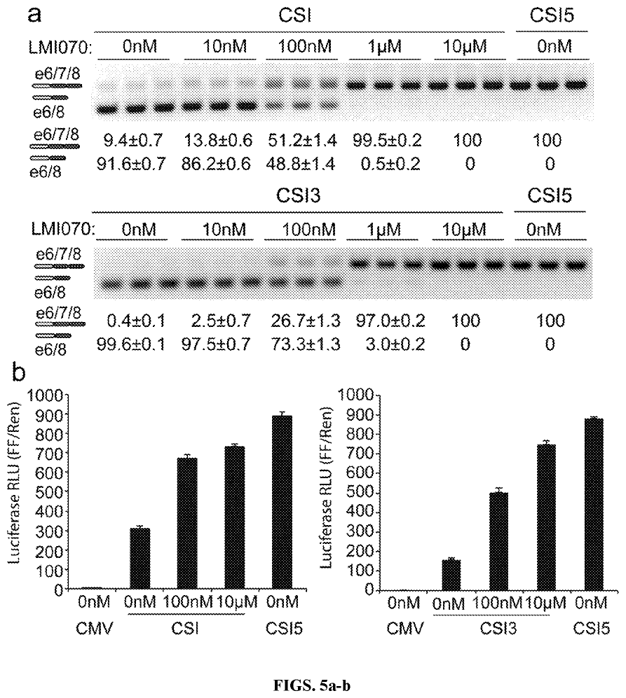

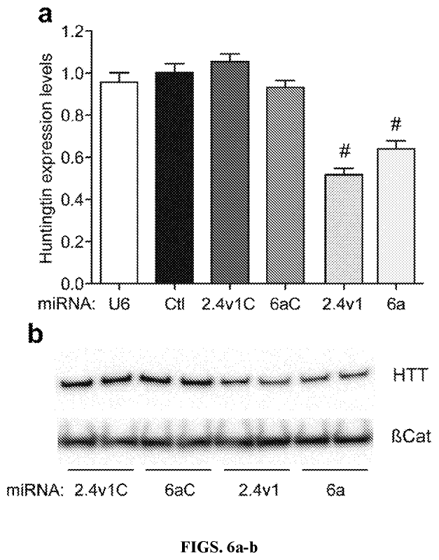

Non-allele specific artificial miRNA sequences targeting either HTT exon 2 (mi2.4v1), or HTT exon 44 (miHDS1v6A) was generated. These miRNA sequences were designed using siSPOTR 4° with a limited off-target profile. In addition, a specific seed-controlled miRNA sequences (mi2.4v1C and miHDS1v6a) was designed that will be used as controls. These miRNA controls do not silence HTT expression but contain the same miRNA seed (5′ nucleotides 2-8) to match mi2.4v1 and miHDS1v6a off-target profiles, respectively (FIGS. 6a-b).

### Example 3

In vitro studies: Medium spiny neurons (MSN) represent 95% of the neuronal cell population in the striatum, are the cell type most affected in HD, and are the main target cell for these studies. Therefore, all in vitro studies employed MSN. MSN cultures can be obtained by direct neural conversion of human fibroblasts from normal or HD patients^{41, 42}

MSN cultures are transduced with rAAV2/1, an AAV serotype that effectively transduces MSN neurons in vivo in the mouse brain, and in vitro in MSN cultures⁴³. Previous reports show that reducing 50% HTT expression in the mouse brain is sufficient to improve disease phenotypes^{8, 11, 12} Therefore, 50% silencing is the bar initially set for this regulated promoter system. MSN cultures will be transduced with increasing doses of rAAV2/1 viruses expressing mi2.4v1, and after LMI070 treatment (1 μM), HTT mRNA levels will be determined by Q-RTPCR. Mock-treated non-transduced and transduced MSN cultures will be used as controls to define basal HTT expression levels, and confirm that mi2.4v1 background levels do not appreciably silence HTT. Cells transduced with AAV2/1 expressing mi2.4v1 under the control of the U6 promoter will be used as a positive silencing control. The goal is to define the AAV therapeutic window in which HTT expression is reduced 50% or more only in the presence of LMI070. Once the effective AAV dose is established, the kinetics of the RNAi expression system in response to LMI070 will be determined by analyzing mi2.4v1 expression at different time intervals and at different LMI070 concentrations.

Since the top 20 endogenous miRNAs are responsible for 75% of the miRNA:mRNA binding sites in the human brain⁴⁴, co-opting the endogenous pathway will be investigated by comparing the expression of mi2.4v1 with respect these 20 top miRNAs and their targeted mRNAs. Total RNA will be extracted from transduced MSN cultures treated or mock-treated with LMI070, and mature miRNA levels will be determined by stem loop Q-PCR, as done previously⁴⁵. In the absence of LMI070, it is expected that mi2.4v1 expression will not interfere with endogenous RNAi regulation, which will be confirmed by analyzing the expression of known endogenous target mRNAs44. Off-target silencing associated with mi2.4v1 will be determined by RNA-seq using total RNA samples obtained from transduced MSN cultures after LMI070 or mock treatment. In addition, transcriptome changes induced by LMI070 or the transactivator alone will be investigated. In those cases, transcriptome changes will be determined using total RNA samples obtained from MSN cultures treated with LMI070, and from transduced MSN cultures expressing only the transactivator protein. Given our initial data, LMI070-regulated miRNA expression is expected to provide at least 50% HTT silencing. Cell toxicity—measured using nutrient withdrawal—is also expected to be minimal⁴⁶. Lack of toxicity will also likely correlate with minimal changes to cellular miRNAs levels, or changes in target mRNA expression.

### Example 4

In vivo regulation: rAAV2/1 virus expressing mi2.4v1 under the control of the regulated promoter system (rAAV.RPmi2.4v1) will be injected in the striatum of N171-82Q transgenic mice, a well-established HD mouse model that expresses the first 171 amino acids of the mutant huntingtin protein in the mouse brain⁴⁷. First, rAAV.RPmi2.4v1 or rAAV.RPmi2.4v1C (Seed-based control RNAi trigger) will be given at 5E10, 2.5E10 and 5E9 vg/hemisphere, (n=10 male mice/group) to determine if there is a dose at which the system is overloaded (too much background HTT silencing). As controls, mice will be injected with rAAV viruses expressing mi2.4v1 or mi2.4v1C under the control of the U6 promoter, a strong Pol3 constitutive promoter. The goal is to determine the AAV and LMI070 dose at which a mi2.4v1 pulse induces mutant HTT suppression by 50% or more, and the time that the peak of suppression is reached. Once the rAAV dose is determined, LMI070 will be administered by oral gavage from 1 to 30 mg/kg 3 weeks after delivery. Note that 30 mg/kg is the maximum dose reported to increase SMN2 exon 7 inclusion in the mouse brain, and 1 mg/kg is the minimal dose with therapeutic effects in a SMN mouse model³¹. Experimental controls are mice expressing mi2.4v1 or mi2.4v1C under the regulated promoter and mice expressing mi2.4v1 under the control of the mouse U6 promoter both given formulation buffer only (mock treated). Mice (n=8 male mice/group) will be sacrificed 24, 48, 72, 96, and 120 hours after LMI070 (or mock) administration. Mi2.4v1 and endogenous miRNA, and mutant HTT expression levels will be assessed in brain lysates. This study will provide relevant data regarding the efficacy of LMI070 to initiate an RNAi pulse and the expression kinetics of the system in vivo. LMI070 pharmacokinetics in mice given LMI070 orally at a 3 mg/Kg dose has, in serum, a Cmax (maximum concentration) of 86 nM and a Tmax (Time to reach Cmax) of 4.3 h, with good distribution in brain (brain:plasma ratio concentration of 1.4). Note that at this concentration (120 nM in the brain), LM1070 induces exon 7 inclusion using either the CSI and CSI3 minigenes (FIG. 5).

Next, frequency of pulsing the HTT-targeting RNAi trigger for a similar level of efficacy will be determined, using the data from the prior study as a guide as to when redosing may be necessary. Mice (n=15 male mice/group) will undergo baseline rotarod testing prior to injection to normalize groups, and then injected bilaterally with experimental or control AAV vectors±LMI070. The accelerating rotarod provides a sensitive measure for detecting motor deficits in HD models. After AAV injection, LMI070 will be administered at the previously established dose to reach 50% or more silencing over a 24 to 48 h period, following with once- or twice-weekly re-administrations. Mice will have additional rotarod tests at 10, 14 and 18 weeks as before¹¹, after which they will be euthanized and brains processed for RNA analyses, biochemical studies, and histology.

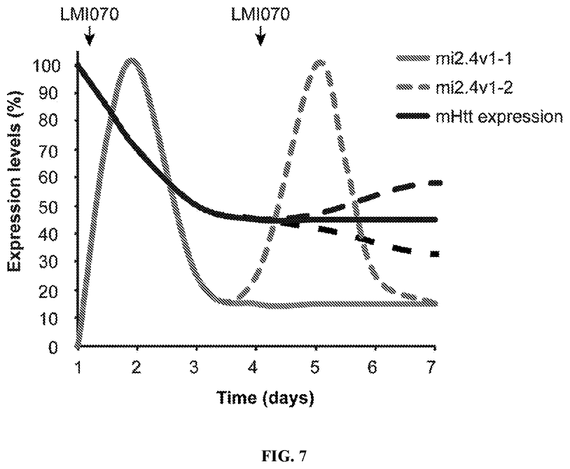

It is expected that the LMI070-induced RNAi pulse will persist for 24-48 hours, reducing mHTT levels ≥50%. Once LMI070 is eliminated, mi2.4v1 levels should decline and mHTT levels will no longer be suppressed (FIG. 7). Prior studies using antisense oligonucleotides molecules (ASO) reported that reversal of disease phenotypes persisted longer than HTT silencing after a transient ASO infusion⁴⁸. Therefore, a pulse of RNAi may need to be maintained for days, or even weeks, similar to what was seen with ASOs targeting mHTT. Since SMN2 exon7 is included in 1% (CSI3) and 10% (CSI) of the SMN2/transactivator transcripts, the RNAi promoter could be partially activated in the absence of LMI070. This could be a problem if artificial miRNAs levels are higher than the transcript levels of the most abundant miRNAs. This issue could be resolved by using a weaker promoter to control transactivator expression (e.g., pGK, mCMV). Based on previous studies LMI070 is not expected to be toxic to mice, but if observed other small molecules have been designed to induce SMN2 exon 7 inclusion that could substitute for LMI070. Because LMI070 can be orally administered, and LMI070 and some rAAV serotypes can cross the blood brain barrier, this system also offers the possibility to develop a less invasive treatment for brain neurodegenerative diseases than current ASO therapies requiring multiple intrathecal administrations.

### Example 5

Prior studies using tissue samples from HD patients and mouse models reported that transcriptome dysregulation is a major event occurring in HD brain, occurring at initial disease stages and before cell loss is observed⁴⁹⁻⁵¹. Recently, it was reported that HD transcriptional changes are not only restricted to the overall expression levels of specific genes, but also to transcript isoforms generated by alternative splicing³². The goal is to identify alternative splicing changes that occur as result of mutant HTT expression and are corrected in response to mutant HTT suppression. This will provide a self-regulating alternative to the SMN2 exon-regulated response element, and will remove the need for drug-related control. Alternative-splicing events that change in response to mutant HTT expression hold promise as regulatory switches to control the production of transactivator in the context of HD. This work can provide a foundation to use a similar approach for controlling expression in other neurodegenerative diseases.

### Example 6

Alternative splicing events that are corrected with mutant HTT silencing: MSN cultures from HD human patients and controls will be transduced with rAAV.U6mi2.4v1 or rAAV.U6miHDS1v6 for silencing HTT expression, and with rAAV.U6mi2.4v1C or rAAV.U6miHDS1v6aC as seed match controls (FIG. 6). Note that by using two different miRNA sequences that effectively target HTT expression and their seed match miRNA controls we will be able to differentiate between alternative splicing events due to mHTT silencing from those events due to RNAi off-targeting.

One week after AAV transduction, when mHTT expression is reduced by 50%, cells will be harvested and total RNA extracted to determine alternative splicing changes. RNA-seq libraries will be prepared using the TruSeq Stranded mRNA Sample prep kit (Illumina) and sent for sequencing using an Illumina HiSeq 4000. RNA-seq reads will be mapped to the Human genome (hg38) and transcriptome (Ensemble, release 89) using STAR software (2.5 or later) allowing up to 3 mismatches per read and up to 2 bp mismatches per 25 bp seed. Cuffdiff will be used to calculate RNA-seq based gene expression using the FPKM metric^{52, 53}. rMATS will be used to identify differential alternative splicing events between the sample groups corresponding to all five basic types of alternative splicing patterns⁵⁴. Significant splicing changes will be considered using a FDR<5% and ΔPSI≥5%. The top 10 alternative splicing events that show the greatest imbalance between transcript isoforms and that are corrected upon silencing of mHTT protein will be validated by PCR on independently obtained samples for biological confirmation.

Then, chimeric eGFP minigenes consisting of the flanking and alternative spliced exons, and intervening minimal intronic sequences will be generated and cloned upstream of an eGFP cDNA in our AAV shuttle vectors. These minigenes will be designed such that eGFP expression will be reduced after mHTT protein repression ‘normalizes’ the splicing profile to that of control MSN cultures. To test the system, MSN cultures will first be transduced with rAAV2/1 expressing miRNAs targeting mutant HTT or the seed match controls, plus rAAVs expressing the eGFP minigenes (or nonmodified eGFP as control). The kinetics of eGFP expression will be determined by western blot and quantitative fluorescence-based assays. Those minigenes responding accordingly to mHTT suppression will then be used to substitute for the SMN2 minigene (FIG. 2), and tested in Tg (N171-82Q) and zQ175 HD mice models.

This disease regulated expression system will allow control of RNAi by taking advantage of alternative splicing changes occurring in response to mHTT expression. It is expected that a significant number of alternative splicing changes will be identified between the HD and control MSN cultures. Alternative splicing changes are not expected to be restricted to exon skipping, but also to the other four types of alternative splicing patterns. Several events with >2 fold imbalance between RNA transcript isoforms are expected to be identified, and those ratios are anticipated to be changed upon silencing of mHTT protein. If no exon skipping splicing events are found that fit the criteria of a >2 fold ratio, this could be solved by introducing specific sequence modifications into the flanking intron/exon constructs, similar to what was done for the SMN2 minigene, to improve ratios that are close but not above that ratio. At the end of this study, a set of disease-regulated minigenes that could be used to substitute for the SMN2 minigene will be obtained.

Examples of differentially spliced introns in HD include MIR4458HG (5: 8457767-8459932), PCDH1 (5: 141869432-141878222), BMP8A (1: 39523698-39523806), TLL1 (4: 166039442-166042026), KCNH1 (1: 210684139-210775347), FGFR1 (8: 38414035-38414151), MGST1 (12: 16606133-16607935), AC097515.1 (4: 16503132-16508540), ATP2B3 (X: 153559943-153560675), RPL22 (1: 6197757-6199564), AC109439.2 (5: 136753973-136754359), SLCO5A1 (8: 69705230-69738039), AC025154.2 (12: 49962381-49963491), SART3 (12: 108539090-108542769), TRPM1 (15: 31067188-31067878), RAIl (17: 17683704-17684064), EMC1 (1: 19233136-19234179), ACYP2 (2: 54115757-54135452), TLL1 (4: 165995179-166003390), HMGCS1 (5: 43298976-43313021), CASTOR3 (7: 100202591-100204020), TRPM1 (15: 31076985-31101656), ABCA8 (17: 68918186-68918426), DMD (X: 31507454-31627672), MACF1 (1: 39084439-39102702), HIPK1 (1: 113957185-113958065), CNTN2 (1: 205062002-205062439), ROBO2 (3: 77644896-77646053), EVC2 (4: 5685480-5689156), WWC1 (5: 168428142-168428706), ISPD (7: 16258483-16258919), PDCL (9: 122823083-122826615), CCDC91 (12: 28255663-28257201), CDK16 (X: 47219106-47222272), ATP2B3 (X: 153543169-153546087), TINAGLI (1: 31585486-31585752), NBPF20 (1: 145401132-145402166), IFT122 (3: 129458678-129460853), MFAPS (12: 8655448-8655785), MED21 (12: 27030273-27030733), COBLL1 (2: 164722523-164727105), MYRIP (3: 40234054-40244445), ARHGAP24 (4: 85827976-85923647), NDST3 (4: 118105106-118114805), ZNF251 (8: 144754746-144755404), LIPM (10: 88815225-88815356), GATD1 (11: 770399-770992), TMEM132C (12: 128696104-128697223), RUBCNL (13: 46378006-46387676), JDP2 (14: 75432337-75437897), NEDD4L (18: 58046019-58165787), ST13 (22: 40844910-4085643), AL662884.4 (6: 32153637-32153998), C6orf118 (6: 165299503-165300363), BDNF-AS (11: 27640006-27658237), ARHGEF7 (13: 111283400-111286146), MYBPC1 (12: 101663561-101667731), RAPSN (11: 47438932-47441158), SMYD1 (2: 88096785-88103057), MRLN (10: 59738563-59738997), KLHL40 (3: 42688718-42688868), TRDN-AS1 (6: 123497253-123503718), VGLL2 (6: 117271065-117272453), ITGA7 (12: 55686315-55687970), LMOD3 (3: 69109122-69118698), VGLL2 (6: 117268492-117270542), ZIM2 (19: 56822837-56823589), KLHL40 (3: 42688303-42688609), TRIM55 (8: 66150467-66152376), COBL (7: 51073386-51085165), RYR1 (19: 38504361-38504747), MYOM1 (18: 3102631-3112297), PDE4DIP (1: 149010596-149012590), NCAM1 (11: 113240834-113242804), TPM3 (1: 154167941-154169304), SMYD1 (2: 88091143-88093516), MYBPC2 (19: 50459007-50459110), TRDN (6: 123218741-123221486), TRDN (6: 123252436-123255080), MICU1 (10: 72508270-72524734), MEF2D (1: 156480778-156482436), ZIM2 (19: 56817580-56817745), SRPK3 (X: 153781146-153781215), COL25A1 (4: 109010376-109048167), STAC3 (12: 57248199-57250992), LMOD3 (3: 69120061-69122092), MAP4 (3: 47912422-47914816), TRIM72 (16: 31214298-31214731), TRIM63 (1: 26066441-26067335), ASB4 (7: 95536551-95537570), CHRND (2: 232528657-232529938), MYPN (10: 68197479-68199367), FBP2 (9: 94559133-94563341), ITGB1 (10: 32901636-32907062), IL17B (5: 149377026-149379204), BVES-AS1 (6: 105162161-105179832), COL23A1 (5: 178239180-178240460), TRDN-AS1 (6: 123438123-123438943), IL17B (5: 149374601-149376735), LINCO1916 (18: 65424072-65441689), AC131025.3 (5: 149329831-149332694), FBP2 (9: 94563462-94567269), MFAPS (12: 8648204-8649500), MEF2D (1: 156479797-156480642), AL358473.2 (1: 201040375-201040621), INPP4B (4: 142086257-142108092), RYR1 (19: 38448512-38448648), MACF1 (1: 39340718-39340803), ITGB1BP2 (X: 71302334-71302410), DUSP13 (10: 75097886-75101866), SAMD8 (10: 75101962-75103893), LSP1 (11: 1884025-1884279), C1orf105 (1: 172465364-172468448), RYR2 (1: 237773649-237778665), TBX18 (6: 84748088-84756697), MYPN (10: 68194513-68195449), TBX18 (6: 84744326-84747919), PYGM (11: 64755559-64757778), MYLK4 (6: 2679409-2680220), LTK (15: 41507291-41507561), DCAF6 (1: 168004794-168015780), LRRFIP2 (3: 37121537-37121634), MFAPS (12: 8654482-8655414), FRMPD1 (9: 37729854-37730983), MYOM2 (8: 2057781-2059152), ADAMTS14 (10: 70674996-70702311), AFAPIL1 (5: 149306405-149307401), GFPT1 (2: 69350184-69354258), UBE4B (1: 10105745-10106196), CCDC141 (2: 178869306-178871426), ITGA4 (2: 181480267-181481597), RGR (10: 84254444-84254696), AL139317.5 (14: 52861035-52861586), ABI3BP (3: 100838285-100838401), AC008429.3 (5: 172956029-172957153), AC004233.2 (16: 2998389-2998493), AC027045.2 (17: 9781595-9791130), ITGB1BP2 (X: 71302557-71303261), EYA4 (6: 133448180-133456555), PRPF18 (10: 13597672-13600243), LINC00592 (12: 52180964-52185378), PPP1R27 (17: 81834654-81834763), MYLK4 (6: 2680292-2683020), MYF6 (12: 80708239-80708523), CLEC3B (3: 45026472-45030826), VCL (10: 74109157-74111908), COL25A1 (4: 108819330-108819811), PHYHD1 (9: 128940498-128940598), ANKRD29 (18: 23601310-23612091), HSPA12B (20: 3748392-3749231), UBE4B (1: 10107375-10117458), AGL (1: 99850899-99850974), CCDC141 (2: 178853625-178855346), AGMO (7: 15418654-15431004), SAMD8 (10: 75108210-75109008), TRPC6 (11: 101471387-101472136), KRT17P5 (17: 18423550-18423656), TMEM241 (18: 23237246-23252959), ENPP1 (6: 131868127-131869357), PHLDB1 (11: 118644692-118645355), MYF5 (12: 80717565-80718357), C12orf42 (12: 103368999-103401606), RBFOX1 (16: 7671600-7676773), CYP2J2 (1: 59916101-59926536), SYPL2 (1: 109478010-109479377), TTN (2: 178799732-178799824), COL6A3 (2: 237395205-237396726), KLHL30 (2: 238148023-238149006), SYNJ2 (6: 158059354-158061991), FNDC1 (6: 159246970-159249038), AC100871.2 (8: 124040007-124045766), CYSLTR1 (X: 78283541-78327304), HSPG2 (1: 21865810-21868884), MET (7: 116775112-116777388), ISCU (12: 108562737-108564059), and PXN (12: 120216582-120216840).

### Example 7

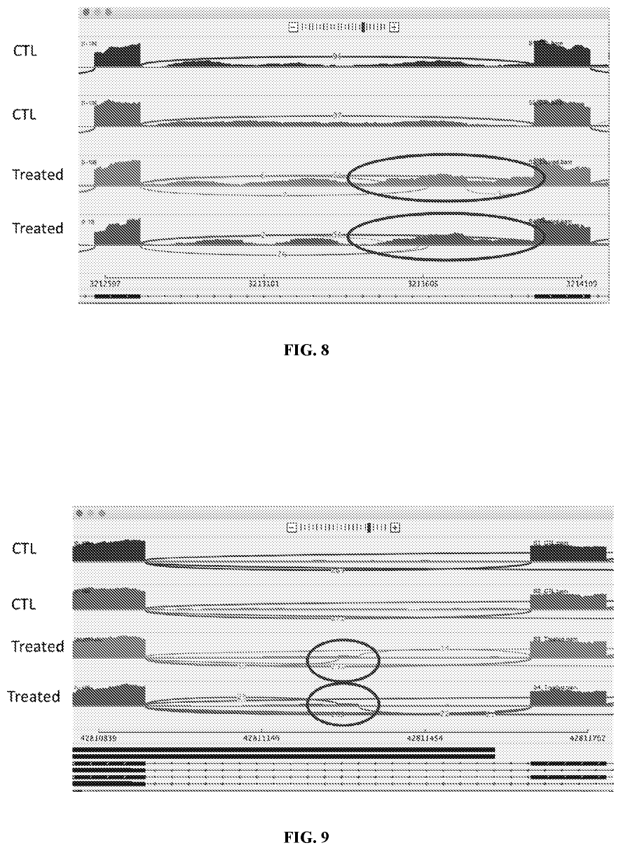

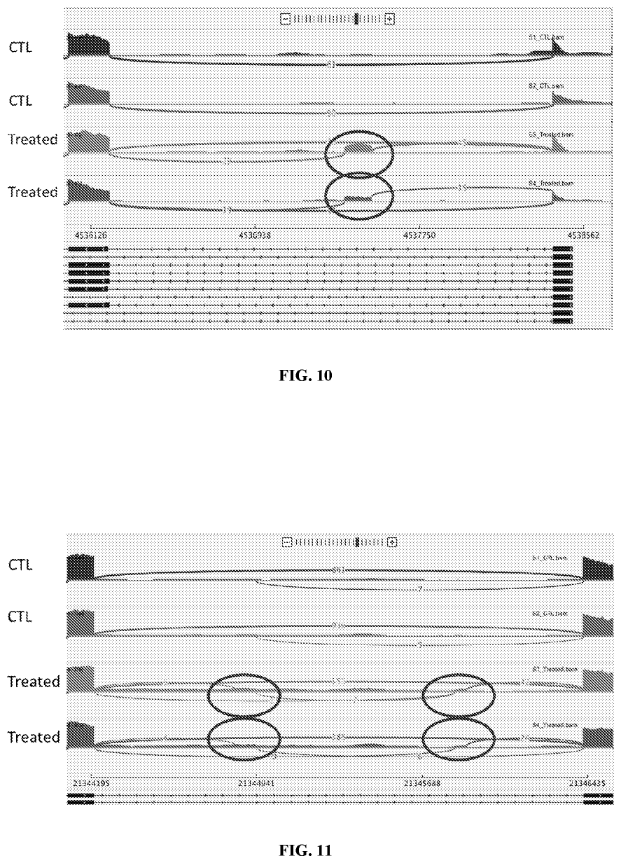

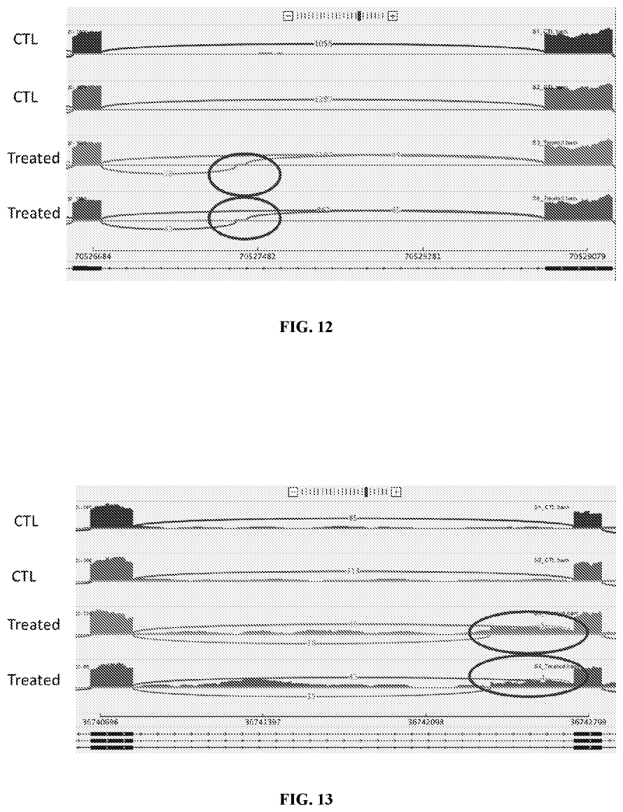

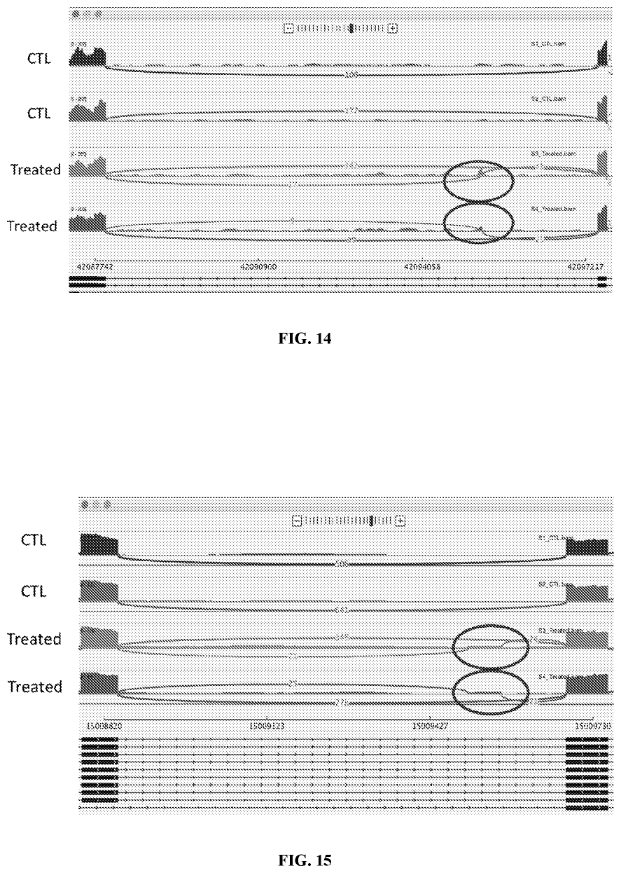

The following methods were used to obtain the data illustrated in FIGS. 8-15. In brief, Hek293 cells were treated with LMI070, at a 25 nM concentration. 12 hours after treatment RNA was extracted using Trizol followed by DNAseI treatment. Samples were evaluated for quality control on agilent bioanalyzer and all samples had RIN values >9.8 and were sent for Illumina RiboZero Gold total RNA library preparation and sequencing with an Illumina HiSeq4000. A 150 bp Pair end sequencing reads were obtained from illumina sequencing. Fastq files obtained from the illumina platform were aligned to the Human genome GRCh38 using the STAR aligner. SAM format alignment files output from STAR were sorted, converted to BAM format and indexed using Samtools. To visualize splicing at any region in the alignment BAM files were opened in IGV and visualized using the Sashimi plot function.

To identify regions of interest which contained high levels of differential splicing a custom R program was created. The input to this custom R program was a matrix of splice junctions output by STAR “.SJ.out.tab”. These files contained the raw counts obtained at each splice junction as calculated during alignment by STAR. This custom R program was created with the goal of identifying highly differentially spliced positions between treatment and control groups. The following steps were used to identify these regions. 1) Read input files into R. 2) Create unique position IDs for each row (splice site) in the dataset. 3). Merge all samples together into one data frame. 4) Calculate the Mean counts, Sum of counts, and Standard deviation of counts for each splice site ID. 4) Replace NA (not available) values with 0. 5) Extract out all splice sites (rows) with sum=0 in the control. 6) sort the extracted reads from highest to lowest by Mean counts value. This yielded a list of splice sites with the greatest level of differential expression with a sum of 0 counts in the control.

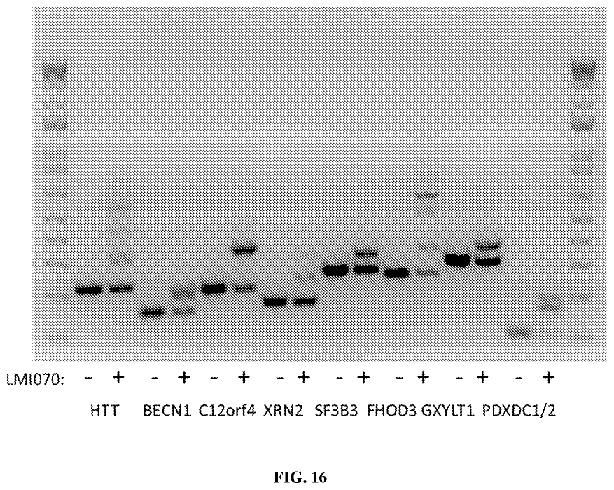

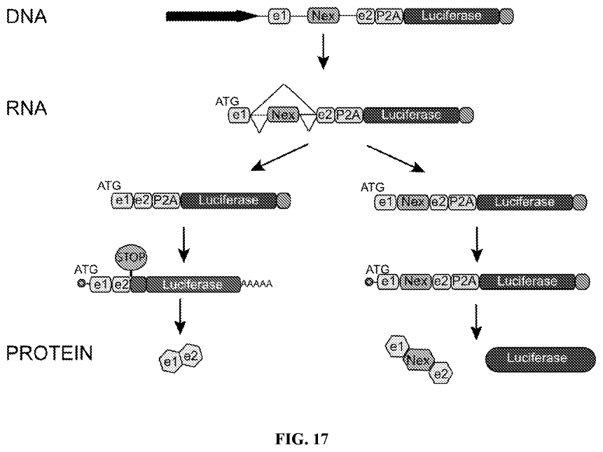

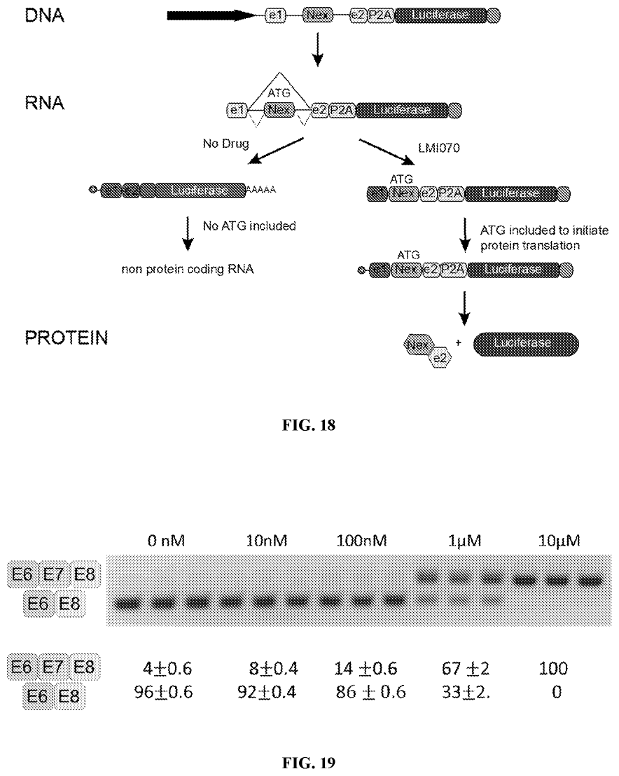

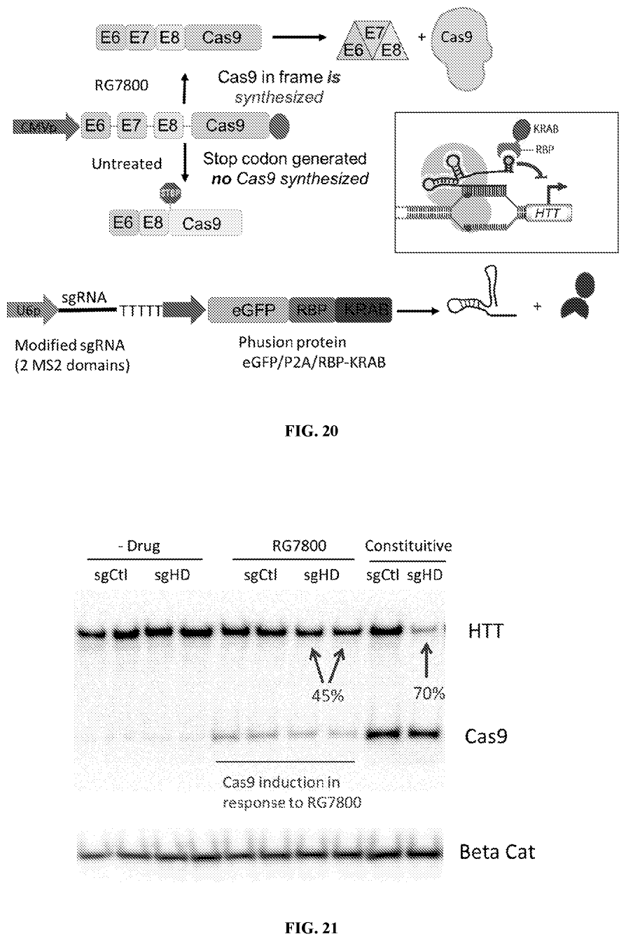

The following methods were used to obtain the data in FIG. 16. The most relevant genes identified in the analysis described above were validated by PCR. RNA was extracted from HEK293 cells treated with LMI070 (25 nM). PCR primers designed to bind upstream and downstream of the novel exon generated by LM1070 treatment were used. As observed on the PCR, PCR products of higher size were detected on samples treated with LM1070 (FIG. 16). These PCR products were sequenced using Sanger sequencing to ensure the inclusion of the novel exon sequence identified.

### Example 8

The following method was used to obtain the data shown in FIG. 19. HEK293 cells were transfected with plasmids expressing the SMN2 minigene (0.3 μg) and 4 hours later treated with different doses of RG7800 (10 nM, 100 nM, 1 μM and 10 μM). 24 hours after transfection, RNA was harvested, DNAseI treated, and 1 μg of RNA was reverse transcribed to assess SMN2 minigene splicing using PCR. PCR products were separated on a 3% agarose gel and exon 7 production was quantified using the ChemiDoc Imaging System (Bio-Rad) and Imagine Lab analysis Software.

The following method was used to obtain the data shown in FIG. 21. HEK293 cells were transfected with plasmids encoding the CRISPRi silencing system (dCas9, sgRNA and MCP-Krab expression cassettes). Importantly, the dCas9 was cloned under the SMN2 splice regulated cassette to control dCas9 protein expression with RG7800. 24 hours after transfection, cells were selected using Puromicin (3 μM, 24 h), passaged onto a new plate, and treated with RG7800 (1 μM). HEK293 cells were lysed 36 h after RG7800 treatment using Passive lysis buffer (Promega, CA), and Huntingtin (HTT), Cas9 and Beta Catenin (Beta cat) protein levels were determined by Western Blot. Beta Catenin protein levels were determined as loading control. HTT, Cas9 and Beta Cat protein levels were quantified using the ChemiDoc Imaging System (Bio-Rad) and Imagine Lab analysis Software.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Heyne et al., “De novo variants in neurodevelopmental disorders with epilepsy,” Nature, 50:1048-1053, 2018.
Lindy et al., “Diagnostic outcomes for genetic testing of 70 genes in 8565 patients with epilepsy and neurodevelopmental disorders,” Epilepsia, 59:1062-1071, 2018.
Shi et al., “Pharmacodynamic assays to facilitate preclinical and clinical development of pre-mRNA splicing modulatory drug candidates,” Pharmacol. Res. Perspect., 3:e00158, 2015.
Singh et al., “Conserved sequences in the final intron of MDM2 are essential for the regulation of alternative splicing of MDM2 in response to stress,” Exp. Cell Res., 215:3419-3432, 2009.
Takeuchi et al., “Splicing Reporter Mice Revealed the Evolutionally Conserved Switching Mechanism of Tissue-Specific Alternative Exon Selection,” PLoS ONE, 5:e10946, 2010.
Zhang et al., “Cell-Type-Specific Alternative Splicing Governs Cell Fate in the Developing Cerebral Cortex,” Cell, 166:1147-1162, 2016.
1. The Huntington's Disease Collaborative Research Group, “A novel gene containing a trinucleotide repeat that is expanded and unstable on Huntington's disease chromosomes.” Cell 72: 971-983, 1993.
2. Walker, F O (2007). Huntington's disease. Lancet 369: 218-228.
3. Zuccato, C, Valenza, M, and Cattaneo, E (2010). Molecular mechanisms and potential therapeutical targets in Huntington's disease. Physiological reviews 90: 905-981.
4. Yamamoto, A, Lucas, J J, and Hen, R (2000). Reversal of neuropathology and motor dysfunction in a conditional model of Huntington's disease. Cell 101: 57-66.
5. Diaz-Hernandez, M, Torres-Peraza, J, Salvatori-Abarca, A, Moran, M A, Gomez-Ramos, P, Alberch, J, et al. (2005). Full motor recovery despite striatal neuron loss and formation of irreversible amyloid-like inclusions in a conditional mouse model of Huntington's disease. The Journal of neuroscience: the official journal of the Society for Neuroscience 25: 9773-9781.
6. Garriga-Canut, M, Agustin-Pavon, C, Herrmann, F, Sanchez, A, Dierssen, M, Fillat, C, et al. (2012). Synthetic zinc finger repressors reduce mutant huntingtin expression in the brain of R6/2 mice. Proceedings of the National Academy of Sciences of the United States of America 109: E3136-3145.
7. Carroll, J B, Warby, S C, Southwell, A L, Doty, C N, Greenlee, S, Skotte, N, et al. (2011). Potent and selective antisense oligonucleotides targeting single-nucleotide polymorphisms in the Huntington disease gene/allele-specific silencing of mutant huntingtin. Molecular therapy: the journal of the American Society of Gene Therapy 19: 2178-2185.
8. Harper, S Q, Staber, P D, He, X, Eliason, S L, Martins, I H, Mao, Q, et al. (2005). RNA interference improves motor and neuropathological abnormalities in a Huntington's disease mouse model. Proceedings of the National Academy of Sciences of the United States of America 102: 5820-5825.
9. Yu, D, Pendergraff, H, Liu, J, Kordasiewicz, H B, Cleveland, D W, Swayze, E E, et al. (2012). Single-stranded RNAs use RNAi to potently and allele-selectively inhibit mutant huntingtin expression. Cell 150: 895-908.
10. Hu, J, Matsui, M, Gagnon, K T, Schwartz, J C, Gabillet, S, Arar, K, et al. (2009). Allele-specific silencing of mutant huntingtin and ataxin-3 genes by targeting expanded CAG repeats in mRNAs. Nature biotechnology 27: 478-484.
11. Boudreau, R L, McBride, J L, Martins, I, Shen, S, Xing, Y, Carter, B J, et al. (2009). Nonallele-specific silencing of mutant and wild-type huntingtin demonstrates therapeutic efficacy in Huntington's disease mice. Molecular therapy: the journal of the American Society of Gene Therapy 17: 1053-1063.
12. Drouet, V, Perrin, V, Hassig, R, Dufour, N, Auregan, G, Alves, S, et al. (2009). Sustained effects of nonallele-specific Huntingtin silencing. Annals of neurology 65: 276-285.
13. Monteys, A M, Spengler, R M, Dufour, B D, Wilson, M S, Oakley, C K, Sowada, M J, et al. (2014). Single nucleotide seed modification restores in vivo tolerability of a toxic artificial miRNA sequence in the mouse brain. Nucleic acids research 42: 13315-13327.
14. Monteys, A M, Wilson, M J, Boudreau, R L, Spengler, R M, and Davidson, B L (2015). Artificial miRNAs Targeting Mutant Huntingtin Show Preferential Silencing In Vitro and In Vivo. Molecular therapy Nucleic acids 4: e234.
15. Ha, M, and Kim, V N (2014). Regulation of microRNA biogenesis. Nature reviews Molecular cell biology.
16. Davidson, B L, and McCray, P B, Jr. (2011). Current prospects for RNA interference-based therapies. Nature reviews Genetics 12: 329-340.
17. McBride, J L, Pitzer, M R, Boudreau, R L, Dufour, B, Hobbs, T, Ojeda, S R, et al. (2011). Preclinical safety of RNAi-mediated HTT suppression in the rhesus macaque as a potential therapy for Huntington's disease. Molecular therapy: the journal of the American Society of Gene Therapy 19: 2152-2162.
18. Grondin, R, Kaytor, M D, Ai, Y, Nelson, P T, Thakker, D R, Heisel, J, et al. (2012). Six-month partial suppression of Huntingtin is well tolerated in the adult rhesus striatum. Brain: a journal of neurology 135: 1197-1209.
19. Birmingham, A, Anderson, E M, Reynolds, A, Ilsley-Tyree, D, Leake, D, Fedorov, Y, et al. (2006). 3′ UTR seed matches, but not overall identity, are associated with RNAi off-targets. Nature methods 3: 199-204.
20. Friedman, R C, Farh, K K, Burge, C B, and Bartel, D P (2009). Most mammalian mRNAs are conserved targets of microRNAs. Genome research 19: 92-105.
21. Jackson, A L, and Linsley, P S (2010). Recognizing and avoiding siRNA off-target effects for target identification and therapeutic application. Nature reviews Drug discovery 9: 57-67.
22. Reyes-Herrera, P H, and Ficarra, E (2012). One decade of development and evolution of microRNA target prediction algorithms. Genomics, proteomics & bioinformatics 10: 254-263.
23. Grimm, D, Streetz, K L, Jopling, C L, Storm, T A, Pandey, K, Davis, C R, et al. (2006). Fatality in mice due to oversaturation of cellular microRNA/short hairpin RNA pathways. Nature 441: 537-541.
24. Boudreau, R L, Martins, I, and Davidson, B L (2009). Artificial microRNAs as siRNA shuttles: improved safety as compared to shRNAs in vitro and in vivo. Molecular therapy: the journal of the American Society of Gene Therapy 17: 169-175.
25. McBride, J L, Boudreau, R L, Harper, S Q, Staber, P D, Monteys, A M, Martins, I, et al. (2008). Artificial miRNAs mitigate shRNA-mediated toxicity in the brain: implications for the therapeutic development of RNAi. Proceedings of the National Academy of Sciences of the United States of America 105: 5868-5873.
26. Giering, J C, Grimm, D, Storm, T A, and Kay, M A (2008). Expression of shRNA from a tissue-specific pol II promoter is an effective and safe RNAi therapeutic. Molecular therapy: the journal of the American Society of Gene Therapy 16: 1630-1636.
27. Le Guiner, C, Stieger, K, Toromanoff, A, Guilbaud, M, Mendes-Madeira, A, Devaux, M, et al. (2014). Transgene regulation using the tetracycline-inducible TetR-KRAB system after AAV-mediated gene transfer in rodents and nonhuman primates. PloS one 9: e102538.
28. Lee, J H, Tecedor, L, Chen, Y H, Monteys, A M, Sowada, M J, Thompson, L M, et al. (2015). Reinstating aberrant mTORC1 activity in Huntington's disease mice improves disease phenotypes. Neuron 85: 303-315.
29. Stanley, S A, Sauer, J, Kane, R S, Dordick, J S, and Friedman, J M (2015). Remote regulation of glucose homeostasis in mice using genetically encoded nanoparticles. Nature medicine 21: 92-98.
30. Reis, S A, Ghosh, B, Hendricks, J A, Szantai-Kis, D M, Tork, L, Ross, K N, et al. (2016). Light-controlled modulation of gene expression by chemical optoepigenetic probes. Nature chemical biology 12: 317-323.
31. Palacino, J, Swalley, S E, Song, C, Cheung, A K, Shu, L, Zhang, X, et al. (2015). SMN2 splice modulators enhance U1-pre-mRNA association and rescue SMA mice. Nature chemical biology 11: 511-517.
32. Lin, L, Park, J W, Ramachandran, S, Zhang, Y, Tseng, Y T, Shen, S, et al. (2016). Transcriptome sequencing reveals aberrant alternative splicing in Huntington's disease. Human molecular genetics 25: 3454-3466.
33. Monani, U R, Lorson, C L, Parsons, D W, Prior, T W, Androphy, E J, Burghes, A H, et al. (1999). A single nucleotide difference that alters splicing patterns distinguishes the SMA gene SMN1 from the copy gene SMN2. Human molecular genetics 8:1177-1183.
34. Scoto, M, Finkel, R S, Mercuri, E, and Muntoni, F (2017). Therapeutic approaches for spinal muscular atrophy (SMA). Gene therapy.
35. Naryshkin, N A, Weetall, M, Dakka, A, Narasimhan, J, Zhao, X, Feng, Z, et al. (2014). Motor neuron disease. SMN2 splicing modifiers improve motor function and longevity in mice with spinal muscular atrophy. Science 345: 688-693.
36. Maeder, M L, Thibodeau-Beganny, S, Osiak, A, Wright, D A, Anthony, R M, Eichtinger, M, et al. (2008). Rapid “open-source” engineering of customized zinc-finger nucleases for highly efficient gene modification. Molecular cell 31: 294-301.
37. Sadowski, I, Ma, J, Triezenberg, S, and Ptashne, M (1988). GAL4-VP16 is an unusually potent transcriptional activator. Nature 335: 563-564.
38. Zhang, M L, Lorson, C L, Androphy, E J, and Zhou, J (2001). An in vivo reporter system for measuring increased inclusion of exon 7 in SMN2 mRNA: potential therapy of SMA. Gene therapy 8: 1532-1538.
39. Cho, S, Moon, H, Loh, T J, Oh, H K, Kim, H R, Shin, M G, et al. (2014). 3′ Splice site sequences of spinal muscular atrophy related SMN2 pre-mRNA include enhancers for nearby exons. The Scientific World Journal 2014: 617842.
40. Boudreau, R L, Spengler, R M, Hylock, R H, Kusenda, B J, Davis, H A, Eichmann, D A, et al. (2013). siSPOTR: a tool for designing highly specific and potent siRNAs for human and mouse. Nucleic acids research 41: e9.
41. Richner, M, Victor, M B, Liu, Y, Abernathy, D, and Yoo, A S (2015). MicroRNA-based conversion of human fibroblasts into striatal medium spiny neurons. Nature protocols 10: 1543-1555.
42. Victor, M B, Richner, M, Hermanstyne, T O, Ransdell, J L, Sobieski, C, Deng, P Y, et al. (2014). Generation of human striatal neurons by microRNA-dependent direct conversion of fibroblasts. Neuron 84: 311-323.
43. Taymans, J M, Vandenberghe, L H, Haute, C V, Thiry, I, Deroose, C M, Mortelmans, L, et al. (2007). Comparative analysis of adeno-associated viral vector serotypes 1, 2, 5, 7, and 8 in mouse brain. Human gene therapy 18: 195-206.
44. Boudreau, R L, Jiang, P, Gilmore, B L, Spengler, R M, Tirabassi, R, Nelson, J A, et al. (2014). Transcriptome-wide discovery of microRNA binding sites in human brain. Neuron 81: 294-305.
45. Monteys, A M, Spengler, R M, Wan, J, Tecedor, L, Lennox, K A, Xing, Y, et al. (2010). Structure and activity of putative intronic miRNA promoters. Rna 16: 495-505.
46. Consortium, HDi (2017). Developmental alterations in Huntington's disease neural cells and pharmacological rescue in cells and mice. Nature neuroscience 20: 648-660.
47. Schilling, G, Becher, M W, Sharp, A H, Jinnah, H A, Duan, K, Kotzuk, J A, et al. (1999). Intranuclear inclusions and neuritic aggregates in transgenic mice expressing a mutant N-terminal fragment of huntingtin. Human molecular genetics 8: 397-407.
48. Kordasiewicz, H B, Stanek, L M, Wancewicz, E V, Mazur, C, McAlonis, M M, Pytel, K A, et al. (2012). Sustained therapeutic reversal of Huntington's disease by transient repression of huntingtin synthesis. Neuron 74: 1031-1044.
49. Luthi-Carter, R, Strand, A, Peters, N L, Solano, S M, Hollingsworth, Z R, Menon, A S, et al. (2000). Decreased expression of striatal signaling genes in a mouse model of Huntington's disease. Human molecular genetics 9: 1259-1271.
50. Sipione, S, Rigamonti, D, Valenza, M, Zuccato, C, Conti, L, Pritchard, J, et al. (2002). Early transcriptional profiles in huntingtin-inducible striatal cells by microarray analyses. Human molecular genetics 11: 1953-1965.
51. Dunah, A W, Jeong, H, Griffin, A, Kim, Y M, Standaert, D G, Hersch, S M, et al. (2002). Spl and TAFII130 transcriptional activity disrupted in early Huntington's disease. Science 296: 2238-2243.
52. Trapnell, C, Williams, B A, Pertea, G, Mortazavi, A, Kwan, G, van Baren, M J, et al. (2010). Transcript assembly and quantification by RNA-Seq reveals unannotated transcripts and isoform switching during cell differentiation. Nature biotechnology 28: 511-515.
53. Trapnell, C, Roberts, A, Goff, L, Pertea, G, Kim, D, Kelley, D R, et al. (2012). Differential gene and transcript expression analysis of RNA-seq experiments with TopHat and Cufflinks. Nature protocols 7: 562-578.
54. Shen, S, Park, J W, Lu, Z X, Lin, L, Henry, M D, Wu, Y N, et al. (2014). rMATS: robust and flexible detection of differential alternative splicing from replicate RNA-Seq data. Proceedings of the National Academy of Sciences of the United States of America 111: E5593-5601.

What is claimed is:

1. A method of providing and/or controlling expression of a protein in a mammalian cell comprising administering to the cell:
(a) a 1^{st} expression cassette comprising a chimeric gene operably linked to a 1^{st} expression control element, wherein the chimeric gene comprises a first portion comprising an alternatively spliced minigene and a second portion that encodes an RNA that encodes the protein, wherein expression of the protein is controlled by the alternative splicing of the first portion.

2. A method of providing a protein to a subject comprising administering to the subject:
(a) a 1^{st} expression cassette comprising a chimeric gene operably linked to a 1^{st} expression control element, wherein the chimeric gene comprises a first portion comprising an alternatively spliced minigene and a second portion that encodes an RNA that encodes the protein, wherein expression of the protein is controlled by the alternative splicing of the first portion.

3. A method of treating a disease in a mammal comprising administering to the mammal:
(a) a 1^{st} expression cassette comprising a chimeric gene operably linked to a 1^{st} expression control element, wherein the chimeric gene comprises a first portion comprising an alternatively spliced minigene and a second portion that encodes an RNA that encodes a protein, wherein expression of the protein is controlled by the alternative splicing of the first portion.

4. The method of claim 3, wherein the second portion that encodes the RNA that encodes the protein includes a translation stop codon, lacks an initiation or start codon, is not an open reading frame to produce the protein, or encodes only a portion of the protein.

5. The method of claim 4, wherein alternative splicing of the first portion modifies the transcript thereby deleting or nullifying the stop codon, introducing an initiation or start codon, restoring the open reading frame, or providing a missing portion of the protein.

6. The method of any one of claims 1-5, wherein the first portion is 5′ of the second portion.

7. The method of any one of claims 1-6, wherein the first portion includes an in-frame translation stop codon.

8. The method of claim 7, wherein alternative splicing of the first portion removes the translation stop codon.

9. The method of any one of claims 1-8, wherein the protein is a transactivator protein.

10. The method of any one of claims 1 and 4-9, wherein the protein is a transactivator protein, wherein the method is a method of controlling expression of an RNA in a mammalian cell, and wherein the method further comprises administering to the cell:
(b) a 2^{nd} expression cassette comprising a nucleic acid sequence encoding the RNA operably linked to a 2^{nd} expression control element that the transactivator protein binds, thereby increasing expression of the RNA in the mammalian cell.

11. The method of any one of claims 2 and 4-9, wherein the protein is a transactivator protein, wherein the method is a method of controlling expression of an RNA in a subject, and wherein the method further comprises administering to the subject:
(b) a 2^{nd} expression cassette comprising a nucleic acid sequence encoding the RNA operably linked to a 2^{nd} expression control element that the transactivator protein binds, thereby increasing expression of the RNA in the subject.

12. The method of any one of claims 3, and 4-9, wherein the protein is a transactivator protein, wherein the method is a method of treating a disease in a mammal, and wherein the method further comprises administering to the mammal:
(b) a 2^{nd} expression cassette comprising a nucleic acid sequence encoding the RNA operably linked to a 2^{nd} expression control element that the transactivator protein binds, thereby increasing expression of the RNA in the mammal and treating the disease.

13. The method of any one of claims 10-12, wherein the RNA is an inhibitory RNA.

14. The method of claim 13, wherein the inhibitory RNA is a siRNA, shRNA, or miRNA.

15. The method of claim 13 or 14, wherein the inhibitory RNA inhibits or decreases expression of an aberrant or abnormal protein associated with a disease, thereby treating the disease.

16. The method of any one of claims 1-15, wherein the RNA encodes a therapeutic protein.

17. The method of claim 16, wherein the therapeutic protein corrects a protein deficiency associated with a disease, thereby treating the disease.

18. The method of any one of claims 1-15, wherein the RNA encodes a Cas9 protein.

19. The method of claim 18, further comprising administering to the subject a 3^{rd} expression cassette comprising a nucleic acid sequence encoding a guide RNA operably linked to a 3^{rd} expression control element.

20. The method of claim 19, wherein the 3^{rd} expression control element is a constitutive promoter.

21. The method of claim 19 or 20, wherein expression of the Cas9 protein and guide RNA corrects a genetic disease.

22. The method of any one of claims 18-20, wherein the Cas9 protein lack nuclease function, wherein expression of the Cas9 protein and the guide RNA inhibits the expression of a gene.

23. The method of any one of claims 1-22, wherein the 1^{st} expression control element is a constitutive promoter, a cell-type specific promoter, or an inducible promoter.

24. The method of any one of claims 1-23, wherein the first portion of the 1^{st} expression cassette and the second portion of the 1^{st} expression cassette are separated by a cleavable peptide.

25. The method of claim 24, wherein the cleavable peptide is a self-cleaving peptide, a drug-sensitive protease, or a substrate for an endogenous endoprotease.

26. The method of any one of claims 1-25, wherein the splicing of the alternatively spliced minigene is regulated by a small molecule splicing modifier.

27. The method of any of claims 1-25, wherein the splicing of the alternatively spliced minigene is regulated by a disease state in a cell.

28. The method of any of claims 1-25, wherein the splicing of the alternatively spliced minigene is regulated by a cell type or tissue type.

29. The method of any of claims 1-28, wherein increased expression of the transactivator or the protein is provided by inclusion of an alternatively spliced exon in the first portion of the chimeric gene.

30. The method of claim 29, wherein the included exon comprises translation initiation regulatory sequences.

31. The method of any of claims 1-30, wherein increased expression of the transactivator or the protein is provided by SMN2 exon 7 inclusion.

32. The method of claim 31, wherein inclusion of SMN2 exon 7 is triggered by the presence of a small molecule splicing modifier.

33. The method of claim 32, further comprising administering the small molecule splicing modifier to the cell or subject, thereby increasing expression of the RNA or the protein.

34. The method of claim 32 or 33, wherein the small molecule splicing modifier is

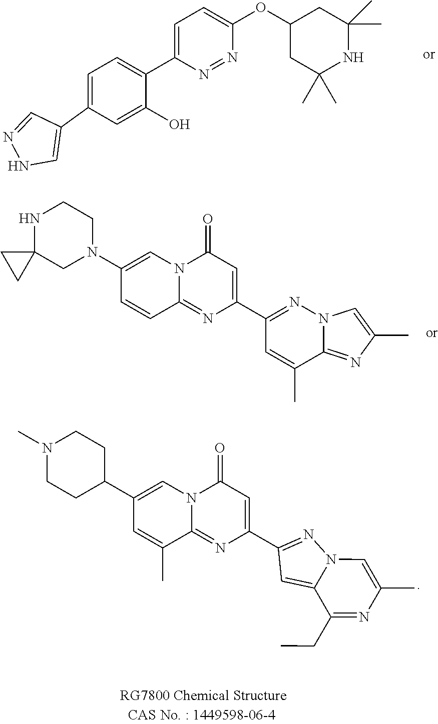

35. The method of any one of claims 1-28, wherein increased expression of the transactivator or the protein is provided by skipping of an alternatively spliced exon in the first portion of the chimeric gene.

36. The method of claim 35, wherein the skipped exon comprises a stop codon.

37. The method of any one of claims 1-36, wherein increased expression of the transactivator or the protein is provided by MDM2 exon 4-11 skipping.

38. The method of claim 37, wherein skipping of MDM2 exon 4-11 is triggered by the presence of a small molecule splicing modifier.

39. The method of claim 38, wherein the small molecule splicing modifier is sudemycin.

40. The method of any one of claims 1-39, wherein the 1P or 2^{nd} expression cassette is comprised in a viral vector.

41. The method of claim 40, wherein the viral vector is selected from an adeno-associated viral (AAV) vector, a lentiviral vector, or a retroviral vector.

42. The method of any one of claims 3-6 and 10-41, wherein the disease is caused by a protein deficiency.

43. The method of any one of claims 3-6 and 10-41, wherein the disease is caused by a genetic defect.

44. The method of any one of claims 3-6 and 10-41, wherein the disease is a neuro-degenerative disease.

45. The method of claim 44, wherein the neuro-degenerative disease comprises a poly-glutamine repeat disease.

46. The method of claim 45, wherein the poly-glutamine repeat disease comprises Huntington's disease (HD).

47. The method of claim 44, wherein the neuro-degenerative disease comprises a spinacerebellar ataxia (SCA), optionally any of SCA1-SCA29.

48. The method of any one of claims 1-47, wherein the mammal is human.

49. The method of any one of claims 2-6 and 8-48, wherein the administration is to the central nervous system.

50. The method of any one of claims 2-6 and 8-49, wherein the administration is to the brain.

51. The method of claim 50, wherein the administration is to the brain ventricle.

52. The method of any one of claims 41-51, wherein the AAV vector comprises an AAV particle comprising AAV capsid proteins and the 1^{st} or 2^{nd} expression cassette is inserted between a pair of AAV inverted terminal repeats (ITRs).

53. The method of claim 52, wherein the AAV capsid proteins are derived from or selected from the group consisting of AAV1, AAV2, AAV3, AAV4, AAV5, AAV6, AAV7, AAV8, AAV9, AAV10, AAV11, AAV12, AAV-rh74, AAV-rh10 and AAV-2i8 VP1, VP2 and/or VP3 capsid proteins, or a capsid protein having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 VP1, VP2 and/or VP3 capsid proteins.

54. The method of claim 52, wherein the one or more of the pair of ITRs is derived from, comprises or consists of an AAV1, AAV2, AAV3, AAV4, AAV5, AAV6, AAV7, AAV8, AAV9, AAV10, AAV 11, AAV12, AAV-rh74, AAV-rh10 or AAV-2i8 ITR, or an ITR having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 ITR sequence.

55. The method of any of any one of claims 1-54, wherein the 1^{st} or 2^{nd} expression cassette comprises a promoter.

56. The method of any one of claims 1-55, wherein the 1^{st} or 2^{nd} expression cassette comprises an enhancer element.

57. The method of any one of claims 1-55, wherein the 1^{st} or 2^{nd} expression cassette comprises a CMV enhancer or chicken beta actin promoter.

58. The method of any one of claims 1-57, wherein the 1^{st} or 2^{nd} expression cassette further comprises one or more of an intron, a filler polynucleotide sequence and/or poly A signal, or a combination thereof.

59. The method of any one of claims 41-58, wherein a plurality of the viral vector are administered.

60. The method of claim 59, wherein the viral vectors are administered at a dose of about 1×10⁶ to about 1×10¹⁸ vector genomes per kilogram (vg/kg).

61. The method of claim 59, wherein the viral vectors are administered at a dose from about 1×10⁷-1×10¹⁷, about 1×10⁸-1×10¹⁶, about 1×10⁹-1×10¹⁵, about 1×10¹⁰-1×10¹⁴, about 1×10¹⁰-1×10¹³, about 1×10¹⁰-1×10¹³, about 1×10¹⁰-1×10¹¹, about 1×10¹¹-1×10¹², about 1×10¹²-×10¹³, or about 1×10¹³-1×10¹⁴ vg/kg of the mammal.

62. The method of claim 59, wherein the viral vectors are administered at a dose of about 0.5-4 ml of 1×10⁶-1×10¹⁶ vg/ml.

63. The method of any one of claims 41-62, further comprising administering a plurality of empty viral capsids.

64. The method of claim 63, wherein the empty viral capsids are formulated with the viral particles administered to the mammal.

65. The method of any of claim 63 or 64, wherein the empty viral capsids are administered or formulated with 1.0 to 100-fold excess of viral vector particles or empty viral capsids.

66. The method of any of claim 63 or 64, wherein the empty viral capsids are administered or formulated with about 1.0 to 100-fold excess of empty viral capsids to viral vector particles.

67. The method of any one of claims 1-66, wherein the delivering or administering comprises intraventricular injection and/or intraparenchymal injection.

68. The method of any one of claims 1-68, comprising administering or delivering it to the brain ventricle, subarachnoid space and/or intrathecal space.

69. The method of any one of claims 1-68, wherein the cells comprise ependymal, pial, endothelial, brain ventricle, meningeal, glial cells and/or neurons.

70. The method of claim 69, wherein the ependymal, pial, endothelial, brain ventricle, meningeal, glial cell and/or neuron expresses the RNA or the protein.

71. The method of any one of claims 3-6 and 10-70, wherein the administration is at a single location in the brain.

72. The method of any one of claims 3-6 and 10-70, wherein the administration is at 1-5 locations in the brain.

73. The method of any one of claims 3-6 and 10-70, wherein the administration is to the: rostral lateral ventricle; and/or caudal lateral ventricle; and/or right lateral ventricle; and/or left lateral ventricle; and/or right rostral lateral ventricle; and/or left rostral lateral ventricle; and/or right caudal lateral ventricle; and/or left caudal lateral ventricle.

74. The method of any one of claims 3-6 and 10-70, wherein the administration is single or multiple doses to any of the mammal's cisterna magna, intraventricular space, brain ventricle, subarachnoid space, intrathecal space and/or ependyma.

75. The method of any one of claims 3-6 and 10-70, wherein the method reduces an adverse symptom of Huntington's disease (HD) or a spinacerebellar ataxia (SCA).

76. The method of claim 75, wherein the adverse symptom comprises an early stage or late stage symptom; a behavior, personality or language symptom; a motor function symptom;
and/or a cognitive symptom.

77. The method of any one of claims 2-6 and 8-76, wherein the method increases, improves, preserves, restores or rescues memory deficits, memory defects or cognitive function of the mammal.

78. The method of any one of claims 2-6 and 8-77, wherein the method improves or inhibits or reduces or prevents worsening of loss of coordination, slow movement or body stiffness.

79. The method of any one of claims 2-6 and 8-78, wherein the method improves or inhibits or reduces or prevents worsening of spasms or fidgety movements.

80. The method of any one of claims 2-6 and 8-79, wherein the method improves or inhibits or reduces or prevents worsening of depression or irritability.

81. The method of any one of claims 2-6 and 8-80, wherein the method improves or inhibits or reduces or prevents worsening of dropping items, falling, losing balance, difficulty speaking or difficulty swallowing.

82. The method of any one of claims 2-6 and 8-81, wherein the method improves or inhibits or reduces or prevents worsening of ability to organize.

83. The method of any one of claims 2-6 and 8-82, wherein the method improves or inhibits or reduces or prevents worsening of ataxia or diminished reflexes.

84. The method of any one of claims 2-6 and 8-83, wherein the method improves or inhibits or reduces or prevents worsening of seizures or tremors seizures or tremors.

85. The method of any one claims 1-84, wherein the mammal is a non-rodent mammal.

86. The method of claim 85, wherein the non-rodent mammal is a primate.

87. The method of claim 86, wherein the primate is human.

88. The method of claim 87, wherein the human is 50 years or older.

89. The method of claim 88, wherein the human is a child.

90. The method of claim 89, wherein the child is from about 1 to about 8 years of age.

91. The method of any one of claims 3-6 and 10-90, further comprising administering one or more immunosuppressive agents.

92. The method of claim 91, wherein the immunosuppressive agent is administered prior to or contemporaneously with administration or delivery of the vector.

93. The method of claim 91, wherein the immunosuppressive agent is an anti-inflammatory agent.
